(19) United States
(12) Patent Application Publication
(10) Pub. No.: US 2009/0104637 A1
(43) Pub. Date: Apr. 23, 2009

(54) Method and Apparatus for Assaying Blood Clotting

(76) Inventors: Rustem F. Ismagilov, Chicago, IL (US); Christian J. Kastrup, Chicago, IL (US); Matthew K. Runyon, Chicago, IL (US); Helen Song, Chicago, IL (US); Feng Shen, Chicago, IL (US)

Correspondence Address: BRINKS HOFER GILSON & LIONE, P.O. BOX 10395, CHICAGO, IL, 60610, US

(21) Appl. No.: 12/162,763

(22) PCT Filed: Jan. 31, 2007
(86) PCT No.: PCT/US07/02532

§ 371 Date: Oct. 31, 2008

Related U.S. Application Data
(60) Provisional application No. 60/763,574, filed on Jan. 31, 2006.

(51) Int. Cl. C12Q 1/56; C12M 1/34

(52) U.S. Cl. 435/13; 435/287.1

(57) ABSTRACT

This invention provides an apparatus for assaying clotting activity. The apparatus includes an inlet for a blood fluid and two or more patches of material in the vessel. The material is capable of initiating a clotting pathway in a blood fluid. This invention also provides an apparatus for measuring clot propagation, which includes a region with material capable of initiating a clotting pathway, and a region where the clot propagation is monitored. Also provided are methods for assaying clotting activity, assaying the integrity of a blood clotting pathway, assaying the effect of a substance on the integrity of a blood clotting pathway, monitoring clot propagation, and preventing clot propagation from one vessel to another.

### CROSS-REFERENCE TO RELATED APPLICATIONS

This invention claims priority to U.S. Provisional Patent Application Ser. No. 60/763,574 filed Jan. 31, 2006.

### GOVERNMENT INTERESTS

This invention was made with United States government support under grant No. CHE 0349034 awarded by the NSF, grant No. N00014-03-1-0482 awarded by ONR PECASE, and grant No. N000140610630 awarded by YIP (Young Investigators Program). The United States may have certain rights in this invention.

### FIELD OF THE INVENTION

This invention is related to the field of methods and devices for assaying blood clotting.

### BACKGROUND OF THE INVENTION

Hemostasis refers to a process whereby bleeding is halted. Hemostasis is the product of a complex biochemical network that controls blood clotting. One of the main functions of this network is to initiate and localize blood clotting at sites of vascular injury. When this network fails to function correctly it can cause excessive bleeding that leads to hemorrhage, or conversely it can result in extensive clot propagation, that leads to thrombosis and, subsequently, to heart attacks and strokes. Thus, initiating blood clot formation in the correct locations and maintaining a localized clotting response are essential to the function of the network. However, the mechanisms regulating this response remain largely uncharacterized and diseases associated with abnormal blood clotting remain the number one cause of death in the United States.

Experiments that are performed to diagnose abnormalities in blood clotting should include the relevant spatiotemporal parameters that exist in vivo. These parameters include: i) heterogeneous surfaces containing the molecules found on the surfaces of blood vessels and in regions of vascular damage, ii) channels that mimic the geometry of blood vessels, and iii) blood flow similar to what is observed in vivo. Clinical experiments that incorporate these parameters would more accurately diagnose diseases associated with blood clotting and may reduce the number of deaths associated with these diseases. However, current clinical experiments used for diagnosing diseases associated with blood clotting do not include these spatiotemporal parameters. These methods include: i) the activated partial thromboplastin time (APTT) test, ii) the prothrombin time (PT) test, and iii) platelet aggregometry. The lack of spatiotemporal parameters these clinical tests may result in misdiagnosis or even lack of diagnosis. Therefore, new clinical methods for diagnosing diseases associated with blood clotting are needed.

### BRIEF SUMMARY

This invention provides an apparatus for assaying clotting activity. In one embodiment, the apparatus includes an inlet for a blood fluid, a vessel in fluid communication with the inlet, and at least two patches in the vessel. Each of the patches includes stimulus material which is capable of initiating a clotting pathway when contacted with a blood fluid from a subject. The stimulus material in one patch differs from the stimulus material in the other patch; or the concentration of stimulus material in the one patch differs from the second patch; or one patch has a surface area different from the other patch; or one patch has a shape different from the other patch; or one patch has a size different from the other patch.

The apparatus may comprise a plurality of patches. In that example, the distance between one set of patches is different from the distance between another set of patches.

The apparatus may include a plurality of patches associated with a surface in the vessel, where a first set of patches is at a first location and a second set of patches is at a second location, and where the number of patches in the first set is different from the number of patches in the second set. The stimulus material may include at least one clotting stimulus selected from the group of tissue factor, factor II, factor XII, factor X, glass, glasslike substances, kaolin, dextran sulfate, bacteria, and bacterial components.

The apparatus may include beads, where the patches are associated with the beads. The apparatus may include patches that are beads. The patch may also include inert material.

The vessel of the apparatus may include two intersecting microchannels, which are in fluid communication with each other.

This invention provides a method of assaying blood clotting. The method includes contacting blood fluid from a subject with at least two patches, where each of the patches includes stimulus material which is capable of initiating a clotting pathway when contacted with a blood fluid from a subject. The stimulus material in one patch differs from the stimulus material in the other patch; or the concentration of stimulus material in the one patch differs from the second patch; or one patch has a surface area different from the other patch; or one patch has a shape different from the other patch; or one patch has a size different from the other patch. The method includes determining which patch initiates clotting of the blood fluid from the subject.

When practicing the method, the stimulus material may be capable of initiating a clotting pathway in blood fluid from a healthy subject. The contact is maintained for a time sufficient for at least the largest patch to initiate the clotting pathway in a blood fluid from a healthy subject. The method can be practiced with first and second patches whose sizes may differ, or the stimulus material in the first and second patches may differ. As well, the concentration of stimulus material in the first and second patches may differ.

The method may also include contacting blood fluid from the subject with a third and fourth patch, where the patches are associated with a surface, and where the distance between the first and second patches differs from the distance between the second and third patches.

The method may be practiced with patches that are each independently associated with a bead. Either the size or the shape of each bead may differ. Also, the method may be practiced where the clotting pathway is a platelet aggregation pathway.

Contacting blood fluid from a subject with a patch may include first contacting a first amount of blood fluid with a first concentration of beads and second contacting a second amount of blood fluid with a second concentration of beads, where each bead independently is associated with a patch comprising a stimulus material and an inert material. Aliquots of blood fluid may be titrated with beads of increasing size. The blood fluid may be contacted with the patches as a continuous stream. Alternatively, the blood fluid may be contacted with the patches as plugs separated by an immiscible fluid. As well, the vessel may be a microfluidic channel.

Determination of which patches initiate clotting may include observing optically. It may include measuring scattering of light.

The method may be practiced with blood fluid that is selected from the group consisting of whole blood and plasma.

The method may include first adding an excess of clotting factor to the blood fluid before contacting the blood fluid with the patches. The method may include adding a test substance to the blood fluid before contacting the blood fluid with the patches. The method may include monitoring the rate of propagation of a blood clot. The method may also include adding blood fluid from a different subject to the blood fluid before contacting the blood fluid with the patches.

This invention provides an apparatus for measuring clot propagation. The apparatus includes one region comprising a stimulus material, and another region in communication with the first region suitable for monitoring the propagation of a clot. When blood fluid is placed in the first region, a clot forms and propagates to the second region.

The apparatus may include a patch comprising the stimulus material. The apparatus may include a microchannel comprising the first and second regions. Alternatively, the apparatus may include a plurality of parallel microchannels, each microchannel comprising the first and second regions.

The apparatus may include at least one set of intersecting microchannels, where the second region is at the intersection of the first set of the microchannels. The apparatus may include a plurality of microchannels and at least two intersections of the microchannels, where the second region is at one of the intersections and where the sizes of the two intersections are different.

This invention provides a method of monitoring clot propagation, which includes the steps of: contacting blood fluid with a first region of an apparatus, the first region comprising a stimulus material, and monitoring clot propagation in a second region of the apparatus, where the second region is in communication with the first region.

### BRIEF DESCRIPTION OF THE DRAWINGS

FIG. 1 is a schematic illustration of the competition between diffusion and reaction, which determines whether initiation of clotting will occur on a given patch.

FIG. 2 shows images and a graph that illustrate measurement of the propagation of a blood clot through a microfluidic channel in the absence of flow.

FIG. 3 shows microphotographs and a graph illustrating how vessel-to-vessel junctions could be used to assess the threshold of blood clot propagation.

FIG. 4 shows graphs depicting numerical simulations for initiation of clotting based on a simple chemical mechanism.

FIG. 5 illustrates the scaling relationship for initiation in the chemical model and blood plasma, showing how the initiation responded to an amount of clotting stimulus, tissue factor (TF).

FIG. 6 shows images and a graph to illustrate how initiation of clotting of human blood plasma responded to the shape of surface patches of identical area.

FIG. 7 shows images that illustrate how numerical simulations of a simplified reaction-diffusion system demonstrated a response to shape.

FIG. 8 shows images and a graph that illustrate how a simplified chemical system constructed to mimic hemostasis responded to the shape of surface patches presenting identical areas of a stimulus.

FIG. 9 is a schematic drawing of the set-up for experiments with the chemical model.

FIG. 10 depicts graphs that illustrate how rate plots of the rate equations are incorporated in the numerical simulation of the modular mechanism.

FIG. 11 is a graph showing how the numerical simulation indicated that the probability of initiating “clotting” in the model exhibits a threshold response to patch size.

FIG. 12 schematically illustrates the microfluidic chambers used in the blood plasma and whole blood experiments.

FIG. 13 illustrates how the amount of acid generated is dependent on the total surface area of the patches.

FIG. 14 illustrates the quantification of the fluorescence intensity profile of pH-sensitive dye in the chemical model on the photoacid surface.

FIG. 15 illustrates the quantification of initiation of clotting of blood plasma.

FIG. 16 illustrates the quantification of initiation of clotting of blood plasma on arrays.

FIG. 17 shows images and graphs that illustrate how human blood plasma and the simple chemical model both initiate clotting with a threshold response to the size of patches presenting clotting stimuli.

FIG. 18 shows images and graphs that illustrate how the chemical model correctly predicts that in vitro initiation of clotting in human blood plasma depends on the spatial distribution, rather than the total surface area of a lipid surface presenting tissue factor (TF), an activator of clotting.

FIG. 19 shows images that illustrate how the chemical model correctly predicts that initiation of clotting of human blood plasma can occur on tight clusters of sub-threshold patches that communicate by diffusion.

FIG. 20 shows images that illustrate how the chemical model correctly predicts initiation of clotting via the second (factor XII) pathway.

FIG. 21 is a schematic drawing of the proposed mechanism for regulation of clot propagation through a junction of two vessels at high (a) and low (b) shear rates.

FIG. 22 is an illustration of how a threshold to \dot{γ} regulates clot propagation through the junction.

FIG. 23 is an illustration of how clot propagation through a junction is regulated by \dot{γ} at the junction and not at the “valve”.

FIG. 24 illustrates how clot propagation through a junction can be changed by adding inhibitors.

FIG. 25 is a schematic of the experimental procedure for monitoring clot propagation through a junction in the presence of flow.

FIG. 26 is a schematic drawing showing actual geometry and dimensions of the devices used for clot propagation through a junction in the presence of flow.

FIG. 27 is a schematic of a plug-based microfluidic device for determining the APTT and for titrating argatroban.

FIG. 28 illustrates merging within a microfluidic device using a hydrophobic side channel.

FIG. 29 illustrates how a hydrophilic glass capillary is inserted into the side channel, and a chart showing how the injection volume into the plug was controlled by flow rate.

FIG. 30 illustrates using brightfield microscopy and a chart of observed clots within plugs of whole blood.

FIG. 31 illustrates using brightfield and fluorescence microscopy images and a chart of the formation of fibrin clots within plugs of platelet-rich plasma (PRP).

FIG. 32 shows graphs that illustrate measurement of thrombin generation and APTT at 23° C. while titrating argatroban into blood samples.

FIG. 33 shows graphs that illustrate APTT measurements at 37° C. while titrating argatroban into (a) normal pooled plasma, (b) donor plasma and corresponding values of the (c) APTT and (d) APTT ratios.

FIG. 34 illustrates an example of a device that can be used to monitor clot propagation of multiple blood samples in parallel in the absence of flow.

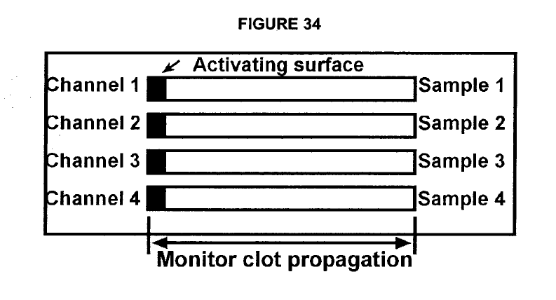

FIG. 35 illustrates an example of a device that can be used to monitor three aspects of clotting: i) initiation, ii) propagation in the absence of a flow and iii) propagation into a flowing blood sample.

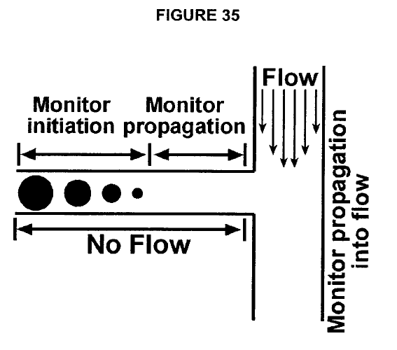

FIG. 36 is a schematic of an experiment to test the hypothesis that the size of individual patches, p, is important, not the total surface area.

FIG. 37 is a schematic of an experiment to test the hypothesis that a cluster of sub-threshold patches will initiate clotting when they are brought close enough together to communicate by diffusion.

FIG. 38 illustrates the schematic of a system capable of rapidly characterizing a person's clotting potential.

### DETAILED DESCRIPTION OF THE PRESENTLY PREFERRED EMBODIMENTS

For the purpose of promoting an understanding of the principles of the invention, reference will now be made to certain preferred embodiments thereof and specific language will be used to describe the same. It will nevertheless be understood that no limitation of the scope of the invention is thereby intended. Any alterations, further modifications and applications of the principles of the invention as described herein are being contemplated as would normally occur to one skilled in the art to which the invention relates.

The coagulation of blood is a complex process during which blood forms solid clots. Blood coagulation is an important part of hemostasis (the cessation of blood loss from a damaged vessel) whereby a damaged blood vessel wall is covered by a fibrin clot to stop hemorrhage and aid repair of the damaged vessel (reviewed in Davie, 2003, J. Biol. Chem. 278: 50819-50832; Nemerson, 1988, Blood 71: 1-8). Briefly, upon blood vessel injury, platelets adhere to macromolecules in the subendothelial tissues and then aggregate to form the primary hemostatic plug. The platelets stimulate local activation of plasma coagulation factors, leading to generation of a fibrin clot that reinforces the platelet aggregate. In the coagulation cascade, the “contact activation” pathway (also known as the “intrinsic” pathway) and the tissue factor pathway (also known as the “extrinsic” pathway) lead to fibrin formation. As wound healing occurs, the platelets aggregate and fibrin clots are broken down. Mechanisms that restrict formation of platelet aggregates and fibrin clots to sites of injury are necessary to maintain the fluidity of the blood.

This invention provides an apparatus (also referred to as “device”) that can be used to measure the clotting time of blood fluid on a surface. The apparatus can be fabricated or manufactured using techniques such as wet or dry etching and/or conventional lithographic techniques or micromachining technology such as soft lithography. As used herein, the term “apparatuses” includes those that are called, known, or classified as microfabricated devices.

In one example, an apparatus according to the invention may have dimensions between about 0.3 cm to about 15 cm per side and thickness of about 1 μm to about 1 cm, but the dimensions of the apparatus may also lie outside these ranges. The apparatus can be made from a variety of materials, and is typically made of a suitable material such as a polymer, metal, glass, composite, or other relatively inert materials. The surface of the apparatus can be smooth or patterned. Different sides of the apparatus can have different surfaces.

In one embodiment, an apparatus of the present invention includes an inlet for a blood fluid, a vessel in fluid communication with the inlet, and at least one patch in the vessel. The patch includes clotting stimulus (also referred to as “stimulus material”) capable of initiating a clotting pathway when contacted with a sample such as blood fluid from a subject. The patch may also include an inert material. The inert material may be mixed with the stimulus material.

The surface of the apparatus can contain blood clotting stimuli, including activators of the extrinsic clotting pathway and activators of the intrinsic clotting pathway.

For example, a surface can include clotting stimulus capable of initiating the extrinsic clotting pathway, such as tissue factor (TF). A surface can include clotting stimulus capable of initiating the intrinsic clotting pathway, such as glass, glasslike substances, kaolin, bacterial components, dextran sulfate, amyloid beta, ellagic acid, and other artificial surfaces.

The clotting stimulus is any surface that is capable of initiating clotting. Surfaces that are well known to initiate clotting include negatively charged surfaces (Gailani and Broze, 1991, Science 253: 909) and surfaces with bound clotting factors (Mann, 1999, Thrombosis and Haemostasis 82: 165). Negatively charged surfaces that are known to initiate clotting include glass, dextran sulfate, and bacterial components (Persson et al., 2003, J. Biological Chemistry 278: 31884). Clotting factors that are known to initiate clotting when bound to surfaces include tissue factor, factor XII, factor X, and factor II (Kop et al., 1984, J. Biological Chemistry 259: 3993; Mann, 1999, Thrombosis and Haemostasis 82: 165). In addition many cells provide surfaces that can act as stimuli (Mann et al., 1990, Blood 76: 1).

The apparatus can contain one type of blood clotting stimulus. Alternatively, the apparatus can contain two or more stimuli. The concentration of each stimulus on the surface can vary. For example, a clotting stimulus can be used at physiological concentrations, pharmaceutically relevant concentrations, supra physiological concentrations, or subphysiological concentrations. Two or more stimuli can be mixed with each other. The stimuli can be in solution. The stimuli can also be in plugs. Techniques for using plugs are described in the following US patents and patent applications, herein incorporated by reference: U.S. Pat. No. 7,129,091 B2; US 2006/0003439 A1; US 2006/0094119 A1; and US 2005/0087122 A1.

One or more stimuli can be mixed with other substances, inert substances, carriers, drugs, etc. For example, in one preferred embodiment, relipidated TF can be used at concentrations from 1 μmol/L to 1000 μmol/L (in 5 to 5000 nmol/L phospholipid vesicles, PCPS). PCPS can be composed, e.g., of 25% phosphatidylserine, PS, from bovine brain, and 75% phosphatidylcholine, PC, from egg yolk. When TF is in vesicle solution, the preferred concentration of TF in the vesicle solution is about 0.10 nM to about 1000 nM. Alternatively, mixed vesicles of DLPC/PS/Texas Redo DHPE (79.5/20/0.5 mole percents) with reconstituted TF at a concentration of 0.1 mg/mL to 100 mg/mL in 1×HEPES-buffered saline/Ca²⁺ buffer can be used. When TF is used in patches, the preferred TF concentration is from about 0.0001 fmol/cm² to about 1.0 fmol/cm². Also, for TF used in patches, a final concentration of 0.01 nM to 1000 nM of TF is preferred.

Patches that include one or more stimuli can be incorporated into the surface of the device, and typically that surface is inert, or largely inert. The concentration of clotting stimuli in the patches can be varied. Thus, the surface of the apparatus can have a plurality of patches with variable shapes, sizes, types of stimuli, and concentrations of stimuli. In one example, a surface is patterned with patches of stimuli of various shapes, with same or different patch areas.

The shape and size of the patches can vary. Three-dimensional considerations of the shape and size of patches include considerations of both the geometry and the dimensions of the patches. In one example, the patches can have shapes that are symmetrical or regular (e.g. circle, square, rectangle, triangle, star, etc.). Alternatively, the patches can be irregular in size and shape. The number and density of patches on a surface can vary. Preferably, about 1% of the surface is covered in patches. The patches may be located on the walls of a microfluidic channel.

In certain embodiments, the apparatus can be manufactured in the form of channels. Preferably, when the apparatus is manufactured in the form of a channel, the apparatus is a microchannel. In other embodiments, the apparatus can have manufactured channels (vessels) into which patches have been integrated. In one embodiment, an apparatus can include two or more interconnected channels that provide fluid communication. The channels can have different dimensions and geometries such as length, width, thickness, depth, and can also have different form of cross-sections, including square, rectangle, triangle, circular cross-section, etc.

In one embodiment, this invention provides an apparatus that includes one or more channels. For example, such an apparatus can be manufactured in the form of a microfluidic device with channels microengineered. When the apparatus has at least one or more channels, the cross-sections of the channels may be equal or unequal. The channels may provide same or different flow rates. The channels may be parallel, at an angle, or the channels may intersect. The channels may have junctions, which may be used to assess clot propagation. Preferably, the junctions are three-way junctions (junctions have three arms), such as a Y junction or a T junction. The arms can provide equal flow rates. Alternatively, the arms can provide different flow rates, in which case one of the arms is generally of a different diameter. Stimuli can be added into the channels, preferably at the junctions.

In yet another embodiment this invention provides an apparatus that includes one or more patches along a channel. The apparatus may also include at least two channels. In this example, patches may be positioned along one or more channels.

In one embodiment, this invention provides an apparatus with continuous flow of sample through an apparatus with at least two channels. In this embodiment, fluid can be flowed through one channel and sample can be introduced via the other channel. For example, the fluid can include additives, clotting stimuli, drugs, or the fluid can be carrier fluid.

In one embodiment, the patch can be on a bead. Alternatively, the bead itself can be a patch. In another embodiment, this invention provides an apparatus with patches on beads that flow through channels with at least one junction.

In one embodiment, there is no flow after the sample is introduced. This can be done, e.g., using a hydrophobic glass capillary. The sample could be introduced without pumping the fluid into the apparatus. Alternatively, the sample can be introduced via injection.

A test substance can be introduced into the apparatus. The effect of the test substance on blood clotting and/or blood propagation can be monitored. The test substance can be a candidate pharmaceutical, a small molecule, an organic or inorganic molecule, a polymer, a nucleic acid, a peptide, a protein, a member of a compound library, a peptidomimetic, etc. A test substance can be added before contacting blood with patches and/or after contacting blood with patches.

In another embodiment, this invention provides an apparatus with one or more channels containing plugs containing various stimuli, and an inlet port for introducing sample into plugs. The apparatus may include at least one junction for promoting clotting.

The apparatus with patches can be manufactured using methods known in the art, for example as described in Zheng et al., 2004, Advanced Materials 16: 1365-1368; Delamarche et al., 2005, Advanced Materials 17: 2911-2933; Sia and Whitesides, 2003, Electrophoresis 24: 3563-3576; Unger et al., 2000, Science 288: 113-116. These publications are herein incorporated by reference in their entirety for all purposes. In one example, the apparatus may be constructed at least in part from elastomeric materials and constructed by single and multilayer soft lithography (MSL) techniques and/or sacrificial-layer encapsulation methods. The basic MSL approach involves casting a series of elastomeric layers on a micro-machined mold, removing the layers from the mold and then fusing the layers together. In the sacrificial-layer encapsulation approach, patterns of photoresist are deposited wherever a channel is desired.

Patches of desired shape can be made by several methods, including but not limited to: 1) Patches can be made by micropattern formation in supported lipid membranes (Groves and Boxer, 2002, Accounts Chem. Res. 35: 149-157); 2) Patches can be made using photolithography. Using photolithography, patches can be made of lipids with reconstituted TF in an inert lipid background (Yee et al., 2004, J. Am. Chem. Soc. 126: 13962-13972; Yu et al., 2005, Advanced Materials 17:1477-1480). Using photolithography patches can also be made of hydrophilic glass in a inert hydrophobic glass background (Howland et al, 2005, J. Am. Chem. Soc. 127: 6752-6765); 3) Patches can be made using Scanning probe lithography (Jackson and Groves, 2004, J. Am. Chem. Soc. 126:13878-13879); 4) Patches can be printed on surfaces using techniques such as inkjet printing or similar techniques that propel tiny droplets onto surfaces (Steinbock et al., 1995, Science 269: 1857-1860); 5) Patches can be made using microcontact printing (Xia and Whitesides, 1998, Annual Review of Materials Science, 28: 153-184); 6) Patches can be associated with beads, patterned using the above or other methods, or may be of a uniform surface composition and not be patterned.

For clotting to occur on surfaces containing one or more clotting stimuli the size of the surface must be larger than a certain threshold size. “Threshold patch size” with respect to blood clotting, according to this invention, refers to the lower limit of patch size at which blood clotting will initiate. Different shapes of patches (e.g. square vs. star) have different threshold, i.e. clotting potential. As well, changing the dimensions of the patch (e.g. length-to-width ratio of a rectangular patch) will result in a different clotting potential. Thus, the patch shape can dictate whether clotting can occur. The patch thickness or depth is generally in the range of about 1 nm to about 1 μm. The patch can also be a bead with widths from about 1 nm to about 1 mm.

To better illustrate this invention, the patch size can be expressed in terms of the largest distance between the two points of the patch that are furthest from each other. For example, the patch size of a patch in the form of a circle equals the diameter of that circle. The patch size of a patch in the form of a square equals the diagonal of that square. Generally, patches useful for practicing the invention have a threshold size of about 0.01 μm to about 500 μm. Preferably, the threshold patch size is less than about 100 μm. It is also useful to express patch size as the area of the patch. This is especially useful for comparing patches of different shapes. Preferably, the area of the patch is from about 1 μm² to about 1 mm².

Patches useful for practicing the invention include patches that are smaller than the threshold patch size; these patches can also be called “sub-threshold” patches. The threshold patch size is dependent on the stimulus concentration, drug concentration, and the blood donor. Preferably, clotting is measured using patches with sizes from about 1 μm to larger than 1 cm. Using nanopatterning techniques one can measure initiation of clotting on the nanometer scale.

A cluster of sub-threshold patches that are brought close together will initiate clotting. The distance between sub-threshold patches at which clotting will occur is approximately the threshold patch size.

For example, for a particular blood sample and stimulus concentration, the threshold patch size may be 75 μm. If so, patches larger than 75 μm will initiate clotting rapidly, whereas patches smaller than 75 μm will not. Patches of 50 μm will not initiate clotting when spaced 250 μm apart, but will initiate clotting when spaced 25 μm apart.

The patches can include a variety of additives, such as one or more labels, reporter molecules, fluorescent molecules, dyes (e.g. pH-sensitive, thrombin-sensitive), microorganisms (e.g. bacteria, viruses), drugs, proteins, metabolites, metal ions, clotting factors, procoagulant factors or drugs, anticoagulant factors or drugs, fibrinolytic factors or drugs, or other compounds. These compounds can be embedded, lyophilized, conjugated, or in any other way attached to the patches. These compounds can be used in certain preferred embodiments of this invention, e.g. in certain assays, for visualization of assays, to test the influence of externally added substances on blood clotting, etc. The concentration of any of these compounds in a given patch can vary. More than one such compound can be added to a patch. Any one of these compounds can be incorporated into one or more patches. Additives can also be used when monitoring clotting in solution.

Changing the concentration of a given clotting stimulus in the patch will change the threshold patch size, in a predictable manner. Also, changing the concentration of a clot-inhibiting drug will effect the threshold patch size, in a particular manner. Using blood fluid from different donors (including donors with unhealthy blood) will give different threshold patch sizes, in a predictable manner. Also, the threshold patch size changes with stimulus concentration and an added drug.

Small patches can initiate clotting if a group of small patches are brought close together. The distance between patches can vary in the range of about 0.01 μm to about 500 μm. Preferably, the distance between patches is less than about 100 μm. The distance between the closest members of a first set of at least two patches may be different from the distance between the closest members of a second set of at least two patches.

While in some embodiments the patches can be used individually, in other embodiments some patches can be used in concert with other patches, whether similar or dissimilar. Therefore, in one embodiment of this apparatus, patches of similar or dissimilar stimuli can be incorporated into an inert background.

The surface with patches can be suspended in solution. As well, surfaces can be formed as particles or beads. Thus, patches useful for practicing the present invention can be associated with particles or beads. Alternatively, the patches can be three-dimensional and take the form of particles or beads. The size and shape of the particles or beads can be varied.

The apparatus of the present invention can be used for a variety of assays, including: (i) assaying blood clotting: (ii) assaying clot propagation; (iii) assaying the integrity of a blood clotting pathway; (iv) assaying the effect of a substance on the integrity of a blood clotting pathway; and (v) assaying for prevention of clot propagation from one vessel to another.

Generally, the methods of the present invention include contacting a sample with a patch described according to the invention. The sample that is assayed is preferably whole blood or blood fluid (blood-containing fluid, e.g. blood plasma), but it can also include blood constituents, solution of plasma proteins, and solution of cells from blood. The sample can be obtained from various subjects, including humans and non-human animals such as rats, mice, and zebra fish. Preferably, the sample is obtained from humans.

The sample can be obtained from a single specimen. Alternatively, the sample can be obtained from multiple specimens. Samples from multiple specimens or multiple subjects can be mixed prior to contacting a patch; alternatively, samples from multiple specimens or multiple subjects can be sequentially brought into contact with the patch. The samples can be obtained from healthy human or non-human subjects. The samples can alternatively be obtained from unhealthy human or non-human subjects. It is also possible to mix the samples obtained from healthy and unhealthy subjects and use that mixture in the assays. As well, it is possible to sequentially add to a patch samples from healthy and unhealthy subjects, in any order.

The sample can include a variety of additives, such as one or more labels, reporter molecules, fluorescent molecules, dyes (e.g. pH-sensitive, thrombin-sensitive), microorganisms (e.g. bacteria, viruses), drugs, proteins, metabolites, metal ions, clotting factors, procoagulant factors or drugs, anticoagulant factors or drugs, fibrinolytic factors or drugs, or other compounds. These compounds can be used in some preferred embodiments of this invention, e.g. in certain assays, for visualization of reactions or blood clot propagation, to test the influence of externally added substances on blood clotting, etc. The concentration of any of these compounds in the sample can vary. Any one of these compounds can be incorporated into one or more samples that are brought into contact with one or more patches. It is also possible to include same or different additives to both a patch and a sample.

The sample is brought into contact with the patch. The sample can be placed on the patch. For example, the sample can be pipetted onto the patch or delivered to the patch using a capillary tube. The sample can be continuously flowed over the surface, thereby contacting one or more patches. Alternatively, the sample can be placed onto the surface where it will contact the patch. As well, the patch can be placed into a sample, so that the sample gets into contact with the patch.

The amount of sample that contacts a patch can vary. Typically, about 20 μl to about 100 μl of sample is used per 1×10⁶ μm² of patch area. Preferably, about 50 μl of sample is used per 1×10⁶ μm² of patch area.

One embodiment of the apparatus of the present invention can be used in a method to measure the potential of a person's blood to clot. The potential can be determined based on the time or likelihood of clotting, where one or more of the following parameters can be varied: stimulus concentration; the size of patches; the concentration of patches; the distance between patches; the shape of patches; the size of particles; the shape of particles; the concentration of patches; the type of stimulus; the flow rate of blood fluid; the concentration of additives, such as drugs, metal ions, clotting factors; and the addition of normal blood fluids. Examples of these are shown below.

In one example, the present invention provides a method for measuring clotting time. Clotting time is measured for a sample that has been brought into contact with the patch. The clotting of blood or blood fluid may be observed optically, as a change in the optical property of the sample, of the patch, or both. In one aspect, the optical property may be a change in color, absorbance, fluorescence, reflectance, or chemiluminescence. The optical property may also be measured at a single or multiple times during an assay. The clotting time may also be detected by measuring scattering of light from the sample, the patch, or both. Clotting time can be compared between samples, or can be compared to clotting time on surfaces that have no patches at all.

The ability of a clot to grow once clotting is initiated can be determined by the velocity of clot propagation on different patches and surfaces, and in different channels (vessels). For example, the speed of propagation of the clot's front can be determined and expressed as distance over time.

Clot propagation can be measured under flow conditions. Alternatively, clot propagation can be measured under no flow conditions.

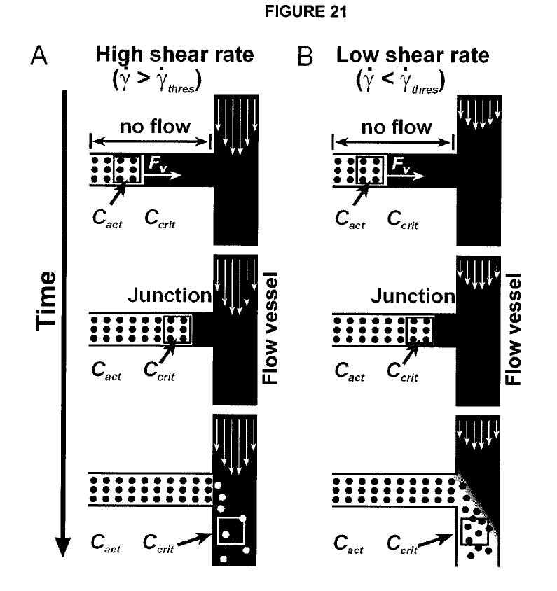

FIGS. 21-26 illustrate regulation of clot propagation through a junction. Clot propagation stops or continues depending on the shear rate, \dot{γ}[s⁻¹], in the vessel with flowing blood (flow vessel) at the junction; also clot propagation through a junction is regulated by the shear rate, \dot{γ}[s⁻¹], at the junction.

Assaying blood clotting can be used for a variety of reasons, including: (i) determining a subject's blood clotting potential; (ii) screening the effect of clotting stimuli; (iii) screening drug candidates that will influence clot initiation, formation, and propagation; and (iv) screening drug concentrations that might influence clot initiation, formation, and propagation.

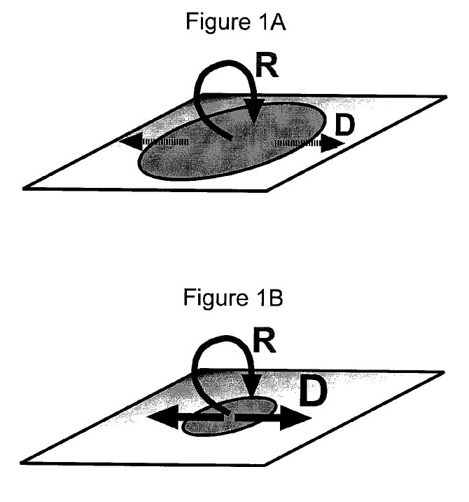

Initiation of blood clotting can be assayed using the methods of the present invention. Initiation of blood clotting displays a threshold response to patch size. In one example, this invention provides a scaling law based on the Damköhler number to describe initiation of clotting on patches of surface stimuli (Kastrup et al., 2006, Proc. Natl. Acad. Sci. USA 103: 15747-15752). Initiation of clotting is thus dependent on competition between the reaction timescale, tᵣ, for production of activators on the patch and the diffusion timescale, t_{D}, for diffusive transport of activators off of the patch (FIG. 1). The magnitude of the Damköhler number, Da=t_{D}/tᵣ, is dictated by the diameter of the patch, p. Small p corresponds to small t_{D} and small Da, as diffusion of activators off of the patch occurs rapidly, whereas large p corresponds to large tᵣ and large Da, as activators take a long time to diffuse from the center to the edge of the patch. Initiation of clotting will occur at large Da when tᵣ is fast and t_{d} is slow. The scaling equation, t=x²/D, relating time, t, distance, x, and the diffusion coefficient for a particular molecule, D, is well established, and can be used to predict the threshold patch size needed to initiate blood clotting, pₜᵣ. On a particular surface with constant tᵣ, the distance molecules of activator will diffuse before reaction occurs should be approximately the same distance as the diameter of pₜᵣ. That is, it takes a certain amount of time for reaction to occur (tᵣ), and at some critical patch diameter (pₜᵣ) molecules can diffuse off of the patch before reaction can occur. Thus pₜᵣ should scale with tᵣ^{1/2} according to

pₜᵣ=(D×tᵣ)^{1/2}

where p is the diameter of the patch, and tᵣ is the reaction timescale.

FIG. 1 illustrates how the competition between diffusion (D arrows), and reaction (R arrows) of activators determines whether initiation of clotting will occur on a given patch (p). The patch in this example is presented as a circle shown in perspective view on square surface. The timescale of diffusion is dependent on patch size, whereas the timescale of reaction is independent of patch size. When the diameter of the patch p is large, reaction out-competes diffusion and initiation will occur. When the diameter of the patch p is small, diffusion quickly removes activator from the patches outcompeting reaction and initiation will not occur.

Among the various applications involving the apparatus and methods according to the invention are observing and measuring threshold responses, including propagating waves and fronts, for the development of diagnostics tools and in drug discovery. Observing and measuring threshold responses could be done using patches, patterned surfaces, or plugs, or by combining one or more patches, patterned surfaces, and plugs.

When measuring the threshold to initiation of blood clotting on patches, this measurement could be done by titrating in beads or particles with different surface chemistry and different sizes of patches containing whole blood or blood plasma and monitoring the dependence of clot initiation on bead/particle composition. For example, the patches may be located on beads suspended in the blood fluid. The aliquots of blood fluid may be titrated with increasing numbers of beads. The aliquots of blood fluid may be titrated with beads of increasing size. The blood fluid may be transported to the patches as a continuous stream. The blood fluid may be transported to the patches as plugs separated by an immiscible fluid.

The present invention provides methods for assaying for clot propagation from one vessel to another based on the shear rate. The shear rate describes the change in the local flow rate, V [m s⁻¹], with increasing distance from a surface. The shear rate determines transport in all directions near a surface. In pressure-driven flows, the local flow rate, V [m s⁻¹], at a surface is zero.

This invention also provides a method for measuring the rate at which clots propagate and how diseases that are related to clot formation and propagation change this rate of blood clot propagation. Such blood coagulation disorders or diseases include hemophilia, inherited bleeding disorder, activated protein C resistance, von Willbrand's disease, and hypercoagulability. Examples of clotting factor deficiencies that are known to slow down clot propagation are factor VIII (fVIII), factor X (fX), and factor XI (fXI) (Ovanesov et al., 2005, J. Thromb. Haemost. 3: 321-331). These factor deficiencies are associated with the following bleeding diseases: deficiency of fVIII results in hemophilia A, deficiency of fX results in Stuart-Prower disease, and deficiency of fXI results in hemophilia C. The methods of this invention may also be used to examine a sample from a subject who is receiving medication that may affect blood clotting.

The present invention can be used to screen for drugs that affect clot propagation. For example, it is possible to add thrombin inhibitors, thrombomodulin, other inhibitors of clotting, or mixtures thereof, to the sample, to the patch, or to both the sample and the patch. As well, the method may include adding thrombomodulin or other inhibitors of clotting to the sample before exposing the blood fluid to the patches. Clotting inhibitors are expected to decrease the clot propagation, and assays according to the present invention can be conducted to better characterize the effect of these compounds. Alternatively, additives to the patch or to the sample can include one or more blood clotting factors. As well, the method may include adding an excess of a clotting factor to the subject's blood fluid before exposing the blood fluid to the patches. Clotting factors are expected to increase the clot propagation, and assays according to the present invention can be conducted to better characterize the effect of these compounds.

The present invention provides a method of assaying the integrity of a blood clotting pathway. The blood clotting pathway may be a platelet aggregation pathway. This invention also provides a method of assaying the effect of a substance on the integrity of a blood clotting pathway.

This invention also provides a method for determining how clots from different blood samples propagate. In addition, this invention provides a method for determining how the presence of blood flow effects blood clot propagation. In one example, this invention provides a method for determining how different channel geometries alter blood clot propagation. Measuring the blood clotting susceptibility of a subject's blood to propagate through junctions of a different size could be used to assess the effectiveness of a particular drug concentration or to detect abnormalities of particular enzymes and proteins involved in the clotting process. The ability of a particular blood sample to propagate through junctions of different sizes will depend on the drug concentration and the activity of particular enzymes in that blood.

This invention also provides a method that can be used to monitor the effects that different drugs and other molecules, and/or variations in the concentration of naturally occurring proteins, have on the rate of blood clot propagation. Measuring the rate of blood clot growth in the presence and absence of specific drugs could be used to determine how well a clot will grow. For example, using the methods of this invention, it is possible to demonstrate that thrombin inhibitors can prevent clot propagation through a junction of channels at below threshold shear rates. Alternatively, patches containing various stimuli and concentrations can be used to test this.

This invention provides a method of assaying for prevention of clot propagation from one vessel to another. The apparatus of this invention can be manufactured with patches in the form of channels, or with patches integrated into the surfaces of fluidic channels that are in fluid communication with each other. The geometries of the channels can be manufactured so that a range of clotting activity can be measured. Samples, such as blood fluid, are then contacted with the patches. The rate of clot propagation through the junctions of channels at below threshold shear rates is then monitored. If desired, various substances can also be added, to further observe the effect of the added substances on clot propagation through the channel junctions.

The present invention has one or more of the following advantages over known methods for assaying blood clotting: a smaller volume of sample can be used; minimal sample preparation due to automated reagent mixing; possibility for real-time observation of initial platelet aggregation and hence clotting time; the speed of mixing is controllable.

It is contemplated that the methods and devices of the invention can be used to detect activity of other biological pathways besides blood clotting. For example, the potential of one's body fluid to initiate an immune response on a patch can be tested. In this example, body fluid samples are contacted with patches that contain one or more antigens (e.g. microorganisms, bacteria, viruses, etc.). Monitoring threshold patch size to initiation can be used to detect things such as the initiation of the immune response in the presence of clusters of bacterial surfaces.

It is contemplated that the methods and devices of the invention can be used to detect activity of biological pathways in samples that include fluids other than blood or blood plasma. For example, the amount of homoserine lactone required to initiate quorum sensing can be tested with solutions containing bacteria. Monitoring threshold patch size to initiation with solutions other than blood can be used to detect things such as the amount of amyloid beta necessary to initiate Alzheimer's disease pathways, the amount of neuronal damage necessary to initiate epileptic seizures, and can be used for the detection of small quantities of bacteria.

It is to be understood that this invention is not limited to the particular methodology, protocols, subjects, or reagents described, and as such may vary. It is also to be understood that the terminology used herein is for the purpose of describing particular embodiments only, and is not intended to limit the scope of the present invention, which is limited only by the claims. The following examples are offered to illustrate, but not to limit the claimed invention.

### EXAMPLES

The scaling prediction for an autocatalytic system using numerical simulations was experimentally tested and verified using human blood plasma. Three-dimensional numerical simulations were used to verify that the scaling prediction is reasonable for a simple, autocatalytic system that is activated on patches of stimuli with rate and diffusion constants on the same scale as those of known blood clotting components. This simple autocatalytic system is based on a modular mechanism for hemostasis proposed by the inventors (Runyon et al., 2004, Angew. Chem. Int. Edit 43: 1531). A simple, autocatalytic system is referred to here as one that exhibits a threshold response, based on competition between high-order autocatalytic production of activators and low-order consumption of activators. This competition between production and consumption creates at least two steady states, one stable and one unstable. The unstable steady state occurs at the threshold concentration, above which production of activators is faster than consumption.

The mechanism consists of three interacting modules: autocatalytic production of activators, linear consumption of activators, and precipitation (or, clotting) at high concentration of activators. Interactions of production and consumption create two steady states in the system, a stable steady state at low concentration of activator, and an unstable steady state at higher concentration of activator. Normally, the concentration of activator remains near the stable steady state, however large perturbations in the concentration of activator will push the system above the unstable steady state where activator will be amplified and initiation of precipitation will occur. Here, the simulations considered this solution phase autocatalytic system over surfaces containing patches of stimuli and the reaction and diffusion of activators from the patch into solution. Simulations were performed using commercial software (FEMLAB, COMSOL, Sweden).

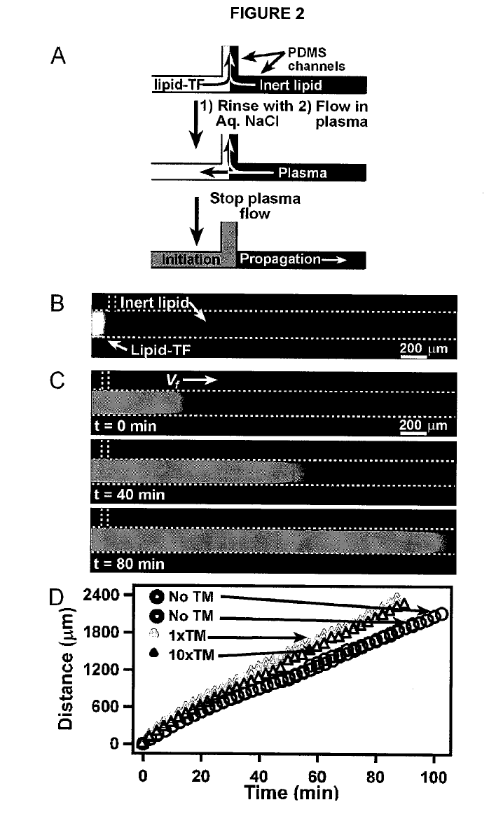

FIG. 2 illustrates continuous and constant clot growth (propagation) throughout a microfluidic channel with no flow. FIG. 2A is an image of a fluorescent micrograph of a microfluidic channel that mimics a damaged blood vessel. In the images, green fluorescence was observed due to a lipid monolayer of PC:Oregon green (inert lipid). Red fluorescence was observed due to a monolayer of DMPC:PS:Texas Red with TF:VIIa complex on the surface (clot activating surface). FIG. 2B illustrates time-lapse fluorescent micrographs of continuous clot growth in a 60×60 μm² microfluidic channel with no flow. Clotting was monitored with a fluorogenic substrate specific for α-thrombin. FIG. 2C is a graph illustrating similar clot growth velocity (V_{f}) in three different channel sizes. In all cases V_{f} was between 30 and 40 μm min⁻¹.

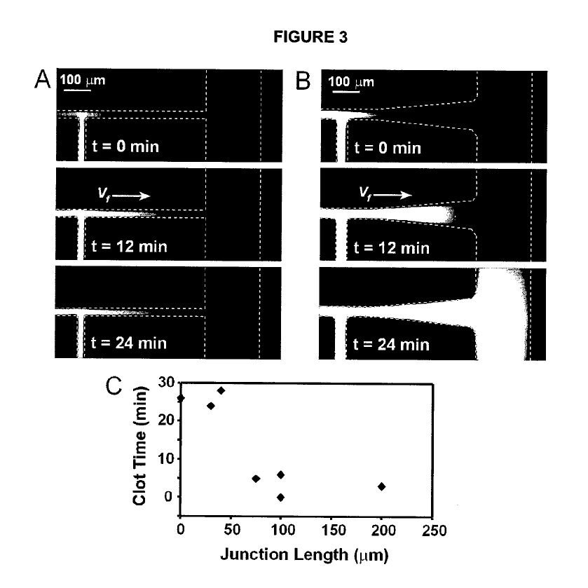

FIG. 3 shows microphotographs illustrating how vessel-to-vessel junctions could be used to assess the threshold of blood clot propagation. FIG. 3A shows time series of clot growth toward a small (20 μm×20 μm) vessel junction. In this microfluidic design the width of the small channel at the junction is below the threshold junction size and clot growth stops. FIG. 3B shows time series of clot growth toward a large vessel junction (100×100 μm×μm). In this microfluidic design the width of the small channel at the junction is above the threshold junction size and clot growth continues into the larger vessel. FIG. 3C illustrates quantification of the threshold junction size for a subject's blood plasma. For this blood plasma the threshold junction size was between 40 μm and 75 μm.

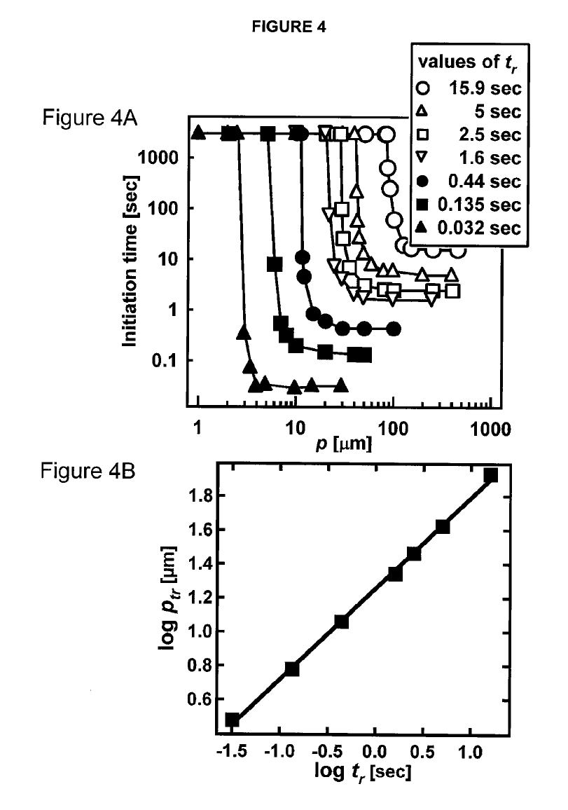

FIG. 4 shows numerical simulations for initiation of clotting based on a simple chemical mechanism. FIG. 4a depicts initiation time vs. patch size curves. Each curve corresponds to a particular tr indicated in the legend. FIG. 4b illustrates how the plot of pₜᵣ vs. tᵣ shows a ½ power scaling relationship and verifies the scaling prediction.

The value of tᵣ for several rates of production from a uniform surface of stimulus was determined. When p was varied for each tᵣ, a threshold patch size was found to exist, as shown in FIG. 4a. For each tᵣ, a specific value of pₜᵣ was observed. When p>pₜᵣ, blood clotting was initiated, and when p<pₜᵣ there was no initiation of blood clotting.

In different sets of experiments, the accuracy of this prediction was tested for a simple, non-linear chemical system. The model was a simple excitable (all-or-nothing) system composed of three reactions. The activator was H⁺. Initiation in this system corresponds to a switch from basic to acid conditions through the significant production of acid from the surface. Acid was produced by irradiating a layer of photoacid molecules on the surface. Patches of acid were produced by selectively irradiating sections of the surface through a photomask. By tuning the intensity of the irradiation and thus the production of acid from the surface, different values for tr were obtained.

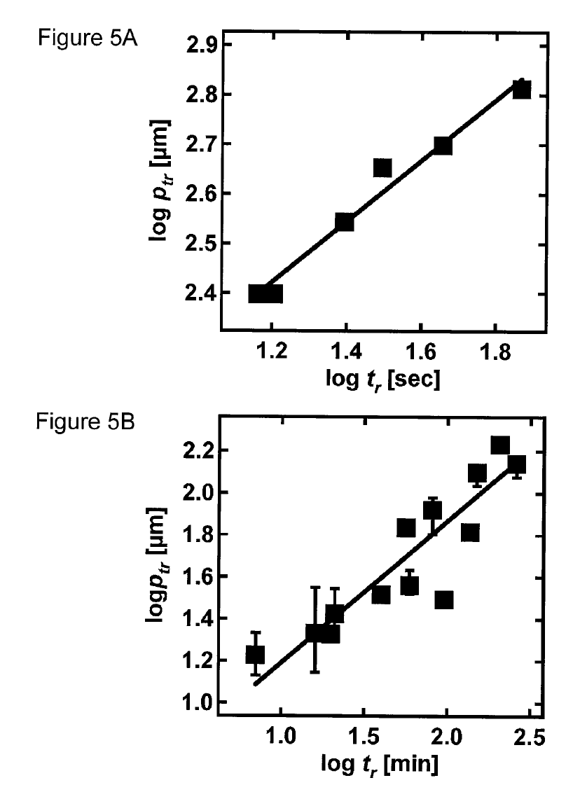

FIG. 5 illustrates the scaling relationship for initiation of blood clotting. Shown in FIG. 5a is the graph of pₜᵣ vs. tᵣ for the chemical model. Shown in FIG. 5b is the graph of pₜᵣ vs. tᵣ for blood samples. For each value of tᵣ, a specific value of pₜᵣ was observed. A plot of pₜᵣ vs. tᵣ showed a ½ power scaling relationship (FIG. 5a) and experimentally verified the scaling prediction.

Blood clotting may be viewed as an excitable system. Initiation in such a system may result in the formation of high concentration of activators such as thrombin and the subsequent formation of a solid clot. The stimulus for production of activators in vivo is the tissue factor (TF). To determine if the scaling prediction applies to blood clotting, the inventors measured the clot times of human blood plasma exposed to surfaces of phospholipid bilayer containing TF. To vary tᵣ in these experiments, the concentrations of TF on the surface and of argatroban, an inhibitor of thrombin in solution, were varied. Patches of TF of specific sizes were obtained through a photolithography process. For each value of tᵣ, a specific value of pₜᵣ was observed. A plot of pₜᵣ vs. tᵣ showed a ½ power scaling relationship (FIG. 5b) and demonstrated the applicability of the scaling prediction to complex and biological systems.

The in vitro experiments of the present invention predict that the size of vascular damage necessary to initiate clotting is related to the timescale of reaction, as described by the Damköhler number. Understanding this relationship will help design better tools to diagnose and treat clotting disorders. Understanding how the concentration of drugs will influence pₜᵣ may be useful for administration of these drugs.

A correct physical description achieved by using the present invention may help predict how susceptible a subject is to blood clotting in vivo. The potential of subject's blood for clotting is routinely determined by measuring clot times in in vitro experiments, where a very high concentration of activator is added at a concentration. These diagnostic methods do not closely mimic the spatiotemporal characteristics of initiation of clotting in vivo, and a better physical description will allow the development of better methods.

The present invention may help understand how activation of all-or-none systems (reactions in complex networks) occurs on surfaces. The present invention may help predict the behavior of complex networks.

### Response to Shape

The inventors demonstrated that response to shape can emerge at the level of a biochemical network. The inventors relied on their developed mechanism (Runyon et al., 2004, Angew. Chem. Int. Edit. 43: 1531) and experimental system (Kastrup et al., 2006, Proc. Natl. Acad. Sci. USA 103: 15747) to examine initiation of coagulation of human blood plasma in vitro. This biochemical network was found to respond to shape—shape of the patch of stimulus controlled whether clotting was initiated.

To characterize the response of initiation of the blood clotting cascade (initiation) to the shape of a patch presenting a stimulus of clotting, the formation of fibrin and the formation of a blue fluorescent dye by thrombin (Lo and Diamond, 2004, Thromb. Haemost. 92: 874) on surface patches of stimuli using bright-field and fluorescence microscopy, respectively, were monitored. The formation of fibrin and thrombin both indicate that clotting has occurred. Surface patches of tissue factor (TF), an integral membrane protein that stimulates initiation, were patterned using photolithography. TF was reconstituted in phospholipid bilayers containing 0.5 mol % of lipid labeled with a red fluorescent dye. Various shapes of the TF surface were presented to human blood plasma in a microfluidic chamber. When comparing patches of different shapes, the area of all patches (and therefore the amount of TF) was kept constant (3.14×10⁴ μm²).

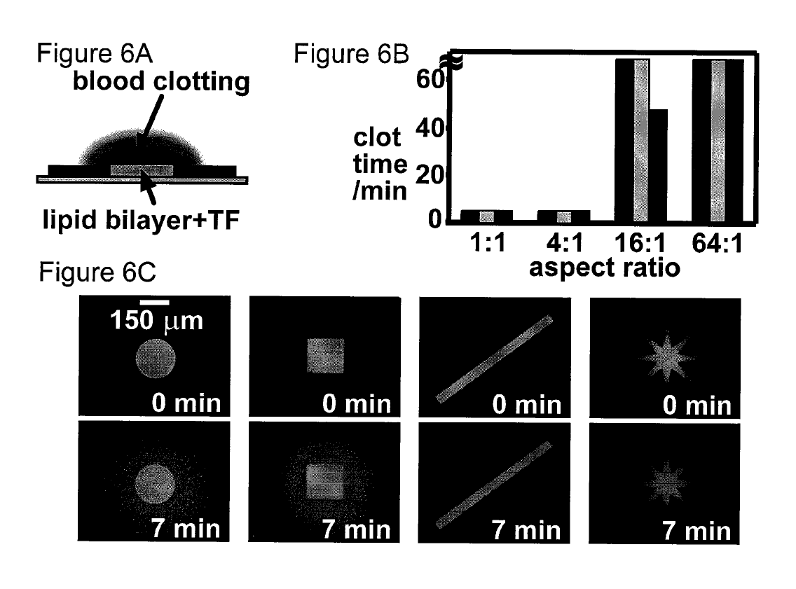

FIG. 6 shows how initiation of clotting of human blood plasma responded to the shape of surface patches of identical area and amount a clotting stimulus, TF. FIG. 6a is a side-view schematic drawing showing clotting on a patch of phospholipid bilayer containing TF. FIG. 6b is a chart quantifying the initiation times of human blood plasma on rectangular patches of varying aspect ratio, measured in triplicate. FIG. 6c shows time-lapse fluorescent micrographs showing clotting on circular and square-shaped patches but not on narrow rectangular and star-shaped patches of the same area.

When human blood plasma was exposed to patches containing TF, initiation only occurred on specific shapes. Initiation occurred on circular patches above a critical size. Initiation on other shapes showed different trends. Wide rectangles, such as a square (aspect ratio=1:1), initiated in less than four minutes, whereas narrow rectangles (aspect ratio≧16:1) did not initiate within 48 minutes (FIG. 6 b, c). From these experiments, it appeared that there is a critical rectangle width necessary to cause initiation (about 90 μm for the experiments above). Interestingly, star-shaped patches were on the border for initiation and initiated in only half of the experiments (seven out of fourteen experiments).

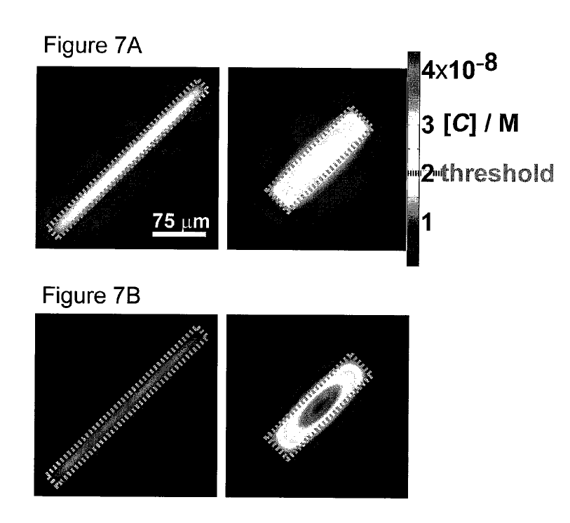

To examine the mechanism behind this response to shape, the inventors developed a 3D numerical simulation that considered a simplified reaction-diffusion system, to reproduce the response to shape in numerical simulations. In the simulation, an autocatalytic reaction mixture was in contact with a surface patterned with patches of stimulus of various shapes with the same area (7854 μm²). This simulation reproduced the experimental results seen in human blood plasma (FIG. 7).

FIG. 7 illustrates numerical simulations of a simplified reaction-diffusion system demonstrated a response to shape. FIG. 7a shows 2D concentration plots from 3D simulations that considered only diffusion and first-order production of activator from a patch showing that [C] was lower on narrow patches. Diffusive removal of activator was more effective on the narrow patch (high aspect ratio, left), maintaining [C] below the threshold, whereas the maximum [C] on the wide patch (low aspect ratio, right) was above the threshold [C]. FIG. 7b illustrates how when solution phase reactions corresponding to second-order autocatalytic production and first order inhibition were also considered, consumption dominated for the narrow patch (left), maintaining [C] below the threshold. Production dominated for the wide patch (right) and [C] increased above the threshold and extensively amplified, resulting in initiation.

To characterize the effects of diffusion on the concentration of activator, [C], on different shaped patches, only first-order production of activator from the patch was considered; reactions in solution were not considered (FIG. 7a). For wider rectangles (lower aspect ratio), the timescale for diffusion from the center of the patch to off of the patch was longer, generating a higher maximum [C] on wide patches than narrow patches (high aspect ratio). To investigate how this difference in [C] between wide and narrow patches affected initiation of an autocatalytic medium, solution-phase reactions were added to the simulation (FIG. 7b). Initiation of this autocatalytic medium had a threshold response to [C] as a consequence of two competing reactions in solution: 1) second-order autocatalytic production of an activator, and 2) first-order consumption, or inhibition, of the activator. Consideration of these solution-phase reactions amplified small differences in [C] between patches, and initiation displayed an all-or-nothing response; [C] either increased several orders of magnitude, resulting in initiation, or remained below the threshold [C], resulting in no initiation. In these simulations, the threshold [C] necessary for initiation was 2×10⁻⁸ M. For a given set of parameters, rectangles with aspect ratios≦4:1 initiated in less than 12 seconds, whereas rectangles with aspect ratios≧16:1 did not initiate within 1000 seconds, the point at which the simulation was stopped.

If this mechanism for the response to shape is correct, a non-biological system based on the same chemical principles as the simulation would reproduce the results seen in human blood plasma. The inventors developed an experimental, chemical model for hemostasis (Runyon et al., 2004, Angew. Chem. Int. Edit. 43: 1531) that reproduced the threshold response to patch area seen in human blood plasma (Kastrup et al., 2006, Proc. Natl. Acad. Sci. USA 103:15747).

The model of the present invention consisted of well-characterized, non-biological reactions that constitute an autocatalytic system based on inhibition and autocatalytic production of an activator, H⁺ (Nagipal and Epstein, 1986, J. Phys. Chem. 90: 6285). In this model, UV light was a stimulus for initiating “clotting”.

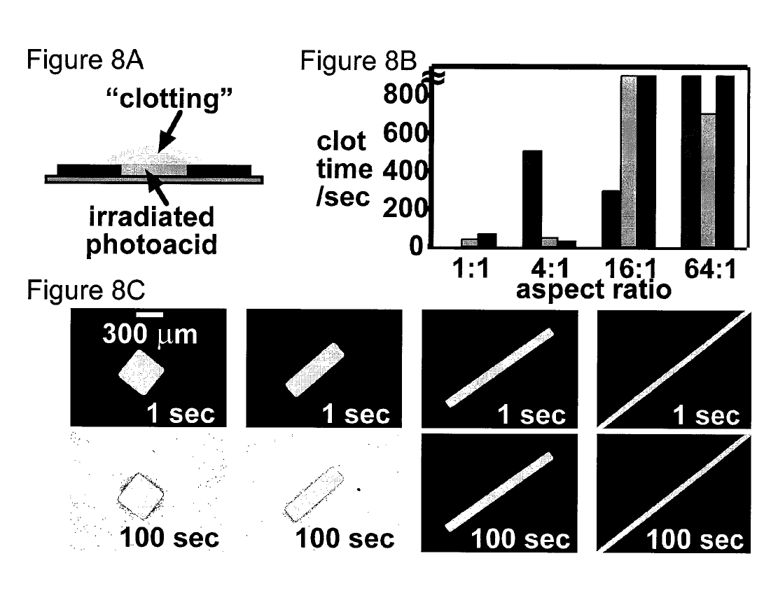

FIG. 8 shows how a simplified chemical system constructed to mimic hemostasis responded to the shape of surface patches presenting identical areas of a stimulus. FIG. 8a is a side-view schematic drawing showing “clotting” on a patch of a photoacid surface irradiated with a UV light stimulus. FIG. 8b is a chart quantifying the initiation times on rectangular patches, measured in triplicate. FIG. 8c shows time-lapse fluorescent micrographs showing that “clotting” occurred on rectangular patches with a small aspect ratio, such as a square, but not on patches with the same surface area and a large aspect ratio.

UV light converted the photoacid, 2-nitrobenzaldyhyde, to 2-nitrosobenzoic acid, and “clotting” occurred when [H⁺] reached the threshold level necessary to induce precipitation of alginic acid from alginate, indicated by a shift of bromophenol blue to yellow (FIG. 8a). As observed in human blood plasma and predicted by simulations, the shape of patches with the same area (1.26×10⁴ μm²) dictated whether or not initiation of this chemical system occurred. Again, initiation was dependent on the aspect ratio of the rectangle (FIG. 8 b, c), where wide rectangles initiated and narrow rectangles did not. Interestingly, in contrast to the results in human blood plasma, stars did not initiate in these experiments. This observation was explained by the numerical simulations. Stars produced concentrations of activators close to the threshold. Changing parameters, such as the rate of production from the patch and the diffusion coefficient of the activator, could shift stars from initiating to not initiating, while other shapes retained the same response.

These results emphasize that while simplified models and simulations capture the overall dynamics of the system, experimental measurements are needed to establish the more subtle details of the dynamics of the complex network. These results further demonstrate that response to shape can emerge not only at the level of an organism, but also at the more basic level of a biochemical network.

### Reagents

All solvents and salts used in buffers were purchased from commercial sources and used as received unless otherwise stated. Poly(dimethylsiloxane) (PDMS, Sylgard Brand 184 Silicone Elastomer Kit) was purchased from Dow Corning. 1,2-dilauroyl-sn-glycero-3-phosphocholine (DLPC), L-α-phosphatidylserine from porcine brain (PS), and 1,2-dipalmitoyl-sn-glycero-3-phosphocholine (DPPC) were purchased from Avanti Polar Lipids. Texas Red® 1,2-dihexadecanoyl-sn-glycero-3-phosphoethanolamine (Texas Red® DHPE), Oregon Green® 1,2-dihexadecanoyl-sn-glycero-3-phosphoethanolamine (Oregon Green® DHPE), N-(7-nitrobenz-2-oxa-1,3-diazol-4-yl)-1,2-dihexadecanoyl-sn-glycero-3-phosphoethanolamine, triethylammonium salt (NBD-DHPE), 5-(and-6)-carboxy SNAFL-1 (SNAFL), rhodamine 110, bis-(p-tosyl-L-glycyl-L-prolyl-L-arginine amide) and FluoSpheres (sulfate microspheres, 1.0 μm, yellow-green fluorescent (505/515), 2% solids) were purchased from Molecular Probes/Invitrogen. Normal pooled plasma (human) (NPP) was purchased from George King Bio-Medical, Inc. t-butyloxycarbonyl-β-benzyl-L-aspartyl-L-prolyl-L-arginine-4-methyl-coumaryl-7-amide (Boc-Asp(OBzl)-Pro-Arg-MCA) was purchased from Peptides International. Albumin (BS) (BSA), and medium viscosity alginic acid were purchased from Sigma. Human recombinant tissue factor (TF) and corn trypsin inhibitor (CTI) were purchased from Calbiochem. Argatroban was manufactured by Abbot Laboratories. Bromophenol blue and sodium chlorite (NaClO₂, 80% purity) were purchased from Acros Organics. Krytox fluorinated grease is a product of Dupont. Siliconized glass coverslips were purchased from Hampton Research. Anhydrous hexadecane, 2-nitrobenzaldehyde, and n-octadecyltrichlorosilane (OTS) were purchased from Aldrich. Sodium thiosulfate (Na₂S₂O₃, 99.9% purity) and anhydrous methyl sulfoxide (DMSO, 99.7% purity) were purchased from Fisher Scientific.

The reagents of the chemical model consisted of solution-phase reagents (the model reaction mixture) and a solid-phase patterned substrate. The model reaction mixture was a solution containing NaClO₂, Na₂S₂O₃, alginic acid, and bromophenol blue (Runyon et al., 2004, Angew. Chem. Int. Ed. 43: 1531-1536). A solution containing NaClO₂ and Na₂S₂O₃ was metastable. By an addition of a threshold concentration of acid (hydronium ion) it could be triggered to react rapidly and autocatalytically, and to produce more acid (Nagypal and Epstein, 1986, J. Phys. Chem. 90: 6285-6292). Alginic acid, under basic conditions, is present as sodium alginate, and is water-soluble. However, under acidic conditions, alginic acid produces an insoluble gel. Bromophenol blue is a pH indicator that was used to monitor the time that the reaction mixture reacts and initiates “clotting”. The reaction mixture was monitored by fluorescence (λₑₓ=535-585 nm, λₑₘ=600-680) of bromophenol blue. When “clotting” was initiated, the basic reaction mixture became acidic, which resulted in the quenching of the red fluorescence and the appearance of a visibly yellow color. The solid-phase patterned substrate consisted of a coverslip coated with a thin layer (20-30 μm) of a dispersion of 2-nitrobenzaldehyde in dimethylsiloxane-ethylene oxide block copolymer. UV-irradiation through a photomask photoisomerized 2-nitrobenzaldehyde (not acidic) to 2-nitrosobenzoic acid (acidic, pKa<4).

Preparing two stable solutions as precursors to the metastable model reaction mixture. Two stable solutions were prepared. When these two solutions were combined, the resulting solution constituted the model reaction mixture, which was metastable. Solution 1 was an aqueous solution of Na₂S₂O₃, alginic acid, and bromophenol blue. Solution 2 was an aqueous solution of NaClO₂.

Preparation of solution 1: The stock alginic acid solution was made by adding alginic acid (0.290 g, medium viscosity) to a solution of NaOH (50 mL, pH=10.8) and was dissolved by heating at about 90° C. for 45 min. The stock Na₂S₂O₃/alginic acid/bromophenol blue solution was made by combining Na₂S₂O₃.5H₂O (0.122 g, 0.492 mmol) and bromophenol blue (sodium salt) (12.5 μl of 0.17 M solution in aqueous NaOH, pH=11.6) in 5 mL of the stock alginic acid solution. This procedure resulted in a Na₂S₂O₃/alginic acid/bromophenol blue solution with a final pH about 7.

Preparation of solution 2: The stock NaClO₂ solution was made by dissolving NaClO₂ (0.270 g, 2.99 mmol) in 10 mL Millipore filtered H₂O (final pH about 10.7). This solution was used within 12 hr.

Combining the reagents to form the metastable reaction mixture used in the chemical model. The model reaction mixture was prepared by combining the stock Na₂S₂O₃/alginic acid/bromophenol and the stock NaClO₂ solutions 1:1 by volume. This procedure resulted in a solution that was initially visibly purple, and also fluoresced in red. Addition of one drop of 1N HCl initiated the “clotting” reaction and turned the solution visibly yellow, also quenching the red fluorescence. Without addition of acid, spontaneous initiation (usually within 20 min) resulted in the same purple to yellow transition due to the stochastic nature of the chlorite/thiosulfate reaction (Nagypal, I. & Epstein, I. R., 1986, J. Phys. Chem. 90: 6285-6292).

Preparing the photoacid-coated substrate. The photoacid, 2-nitrobenzaldehyde, was kept in the dark at all times. The photoacid was dissolved into dimethylsiloxane-ethylene oxide block copolymer (1:1 by weight) by heating to 60° C. with stirring. This mixture was maintained at 60° C. until spin-coated. The homogeneous photoacid/siloxane mixture was spin-coated by placing 50 μL of warm mixture in the center of a siliconized coverslip (22 mm diameter) at room temperature. The substrate was immediately spun at 500 rpm for 10 sec, then at 1500 rpm for 15 sec. Within 5 min, 2-nitrobenzaldehyde solidified out of the siloxane fluid yielding a thin gel-like layer (20-30 μm thick) over the coverslip. The photoacid-coated substrates were kept in the dark and used within 12 hrs.

### Measuring Initiation of “Clotting” in the Chemical Model in a Microfluidic Chamber

Designing and assembling the chamber. The microfluidic chamber used in the chemical model experiments was constructed by sealing a PDMS gasket to a siliconized coverslip. The disposable chamber had an inner diameter of 10 mm, an outer diameter of 20 mm, and a depth of 1 mm. A 30 μL drop of the model reaction mixture was placed in the chamber. The glass coverslip coated with photoacid substrate was placed on top.

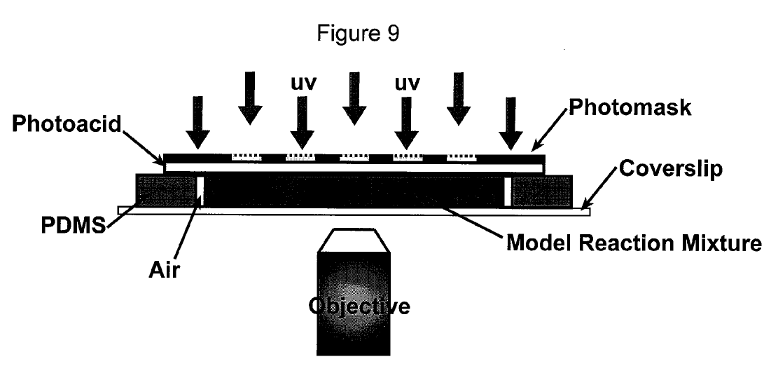

FIG. 9 is a schematic drawing of the set-up for experiments with the chemical model. A PDMS gasket (PDMS) was sealed to a siliconized glass coverslip. The chemical model reaction mixture (30 μL, Model Reaction Mixture) was placed in the chamber. A photoacid layer (20-30 μm) of a dispersion of 2-nitrobenzaldehyde (50% by weight) in dimethylsiloxane-ethylene oxide block copolymer was placed on top of the PDMS and in contact with the chemical model reaction mixture. A photomask (Photomask, black) was placed on top, allowing UV light (300-400 nm, UV arrows) to pass only in specific locations (gray).

Creating acidic patches by UV irradiation. A 100 W Hg lamp was used to irradiate the sample from above. Light passed through a heat absorbing filter (50 mm diameter Tech Spec™ heat absorbing glass) and then through a short-pass filter (Chroma #D350), allowing primarily 300-400 nm wavelengths to reach the sample. Light then passed through a condenser, which was defocused to yield a uniform illumination area of about 6 mm in diameter on the sample. UV light was illuminated through a “silver on Mylar” photomask (CAD/Art Services Inc.) placed directly on top of the glass coverslip coated with the photoacid dispersion.

Imaging the model reaction mixture using epi-fluorescence microscopy. A 150 W Xenon light source was used to monitor the model reaction mixture from below the sample. Light passed through a filter cube (λₑₓ=535-585 nm, λₑₘ=600-680) and a 5×0.15 NA objective. Exposure times of 10 ms were taken every 180 ms, with the camera set al bin=2×2, and gain=255. The quenching of red fluorescence indicated that the model reaction mixture had reacted and initiated “clotting.” Significant photobleaching was not seen for the pH-sensitive dye. When the initiation of “clotting” occurred (after about 22 s of irradiation for large patches), quenching of fluorescence intensity occurred rapidly, decreasing by a factor of about 10 in <1 sec. This is not consistent with simple photobleaching.

The images of the acidic patches in the chemical model system (here the inventors are not referring to monitoring of “clotting”) were obtained by filtering the “UV irradiation source” through a green-pass filter (HOYA). Green light passed through the photomask and the experimental setup to an objective below. Images of the patches were taken from below the sample (see FIG. 9). An image taken from below shows patches that appear “fuzzy” due to the distortion of light as it passed through the thin layer of the solid suspension of the photoacid.

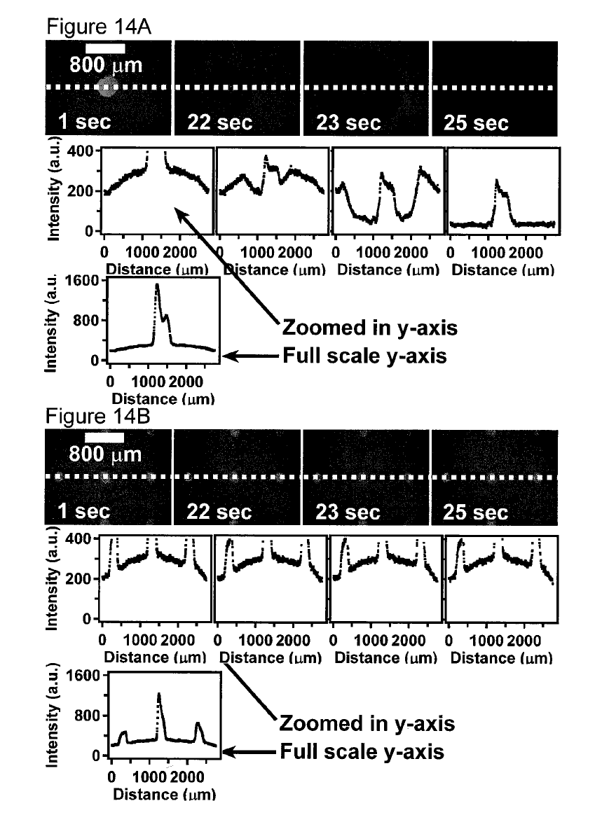

Analyzing images of initiation of “clotting” in the chemical model system. For the model reaction mixture, the original grayscale time-lapse fluorescence images showed a quenching of fluorescence (transition from high fluorescence to low fluorescence) when “clotting” was initiated (see FIG. 14 for images). In MetaMorph® these images were uniformly false colored yellow and thresholded for dark objects. This procedure resulted in an inversion of light yellow and dark areas in all images. The end result was images going from dark to light yellow when “clotting” was initiated. This procedure allowed the use of more sensitive fluorescent imaging, while obtaining the yellow color visually observed upon “clotting”.

The original images of the acidic patches were false colored to green and the levels were adjusted in MetaMorph®. The processed MetaMorph® images were opened in a new Adobe Photoshop document set to RGB mode. An overlaid image was created consisting of two layers: the top layer was the green image of the patch and bottom layer was the yellow image of the “clotting” solution. The blending options for the top layer were set to blend only if green.

Quantifying Acid Production from Patches in the Chemical Model Using 5-(and 6)-Carboxy-Seminaphthofluorescein-1 (SNAFL)

Fabricating an experimental setup to quantify acid production. An experimental set-up similar to that described above for the chemical model was used (same illumination setting and imaging settings). The following differences were applied: 1) a different chamber was used, 2) a 40×0.85 NA objective was used, and 3) the model reaction mixture was replaced by a SNAFL solution. For these experiments, the chamber consisted of 100 μm diameter silver wire wound in a circle about 3 mm diameter, and placed on top of a siliconized coverslip (22 mm). Silicon grease was applied around the wire. A 2 μL drop of 10 μM SNAFL (red fluorescence=basic, green fluorescence=acidic) in 10 mM tris(hydroxymethyl)aminomethane (Tris, pH=9.7) was placed in the silver wire circle, but did not contact the wire. The photoacid substrate was placed on top of the silver wire and sealed down by the silicon grease. The photomask was placed on top of the photoacid substrate.

Generating an acid calibration curve with SNAFL. A calibration curve was generated for fluorescence intensity of SNAFL vs. concentration of acid added. SNAFL/Tris solutions were prepared with varying amounts of HCl added. The final pH of the solutions ranged from 6.5 to 9.7. The green and red fluorescence intensities were measured for the SNAFL/Tris+HCl solutions in the chamber. The calibration curve (ratio green/red intensity vs. [H₃O⁺]) for this acid titration was fitted with a sigmoidal curve.

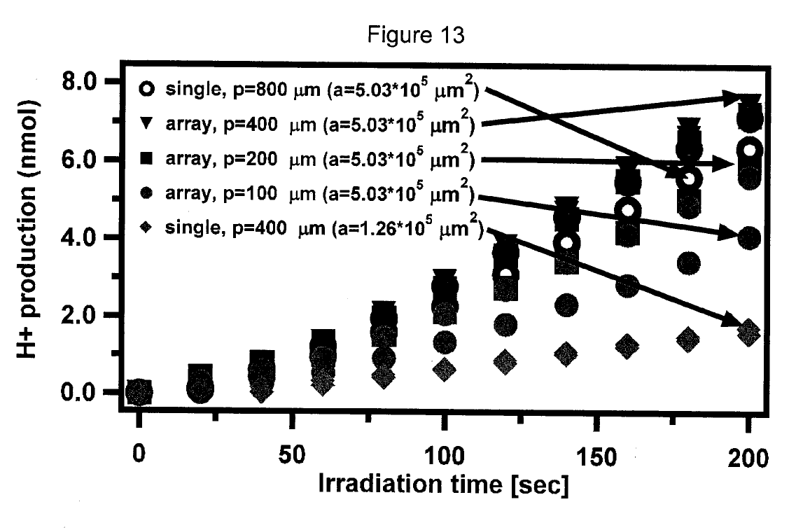

Quantifying acid production for different patch sizes. The acid production of arrays and single patches was measured using the experimental set-up described for the SNAFL solution. Samples were irradiated with a UV pulse for 20 sec, allowed to equilibrate for 2 min, and then the green and red fluorescence intensities were measured. The amount of acid produced was determined using the fluorescence intensity data, the measured calibration curve, and the known volume of the sample (see FIG. 13 for results).

### Numerical Simulations of the Modular Mechanism of Initiation of Clotting on Surfaces Presenting Clotting Stimuli

Numerical simulations were used to illustrate that a threshold patch size can exist for the proposed modular mechanism, using a single rate equation to represent the kinetics of each module. In this example, the inventors: i) tested if competition between two modules, one producing an activator (autocatalytically) and one consuming the activator (linearly), could produce a threshold response to concentration of the activator; ii) tested if a simulation incorporating these two modules, a surface patch that produced activators, and diffusion, could produce a threshold response to the size of the patch; iii) tested if reasonable parameters for biochemical reactions of blood clotting could produce a threshold patch size of the same magnitude as the experimentally measured value. The purpose was not to predict the exact size of the threshold patch. The timescale of reaction, t_{R}, a single experimentally determined parameter, is a simpler and more reliable predictor of the size of the threshold patch.

Choosing parameters used in numerical simulations. In the modular mechanism, the diffusion and reactions occurring at a patch presenting “clotting” stimuli were numerically simulated using a commercial finite element package FEMLAB version 3.1 (Comsol, Stockholm, Sweden). The surface consisted of patches presenting “clotting” stimuli, and a 1 mm “inert” vicinity around the patch. The effect of varying patch size on concentration profiles and “clot time” was determined.

To simulate numerically the change in concentration of activator, “C”, diffusion in solution was considered, as well as reactions occurring in solution and on a surface patch. C may be compared to the set of clot-promoting molecules present in blood. The mass transport of C was modeled with the standard convection-diffusion equation. A diffusion coefficient 5×10⁻¹¹ m²s⁻¹ was used (approximate value for a solution-phase protease in blood clotting, such as thrombin). Convective flow was not used in the simulation. A boundary layer thickness of 1 μm was chosen. For this boundary layer thickness, the assumptions are that lateral diffusion through the layer is fast and that the solution is laterally homogeneous. The size of the boundary layer is rather arbitrary, and a range of thicknesses may be used, as long as diffusion through the thickness of the boundary layer is much faster than the rate of reactions and the rate of diffusion across the smallest patch. The boundary layer is used to simplify 3D simulation to a computationally more efficient pseudo-2D simulation. A boundary condition of insulation/symmetry was used at the outer edge of the “inert” vicinity.

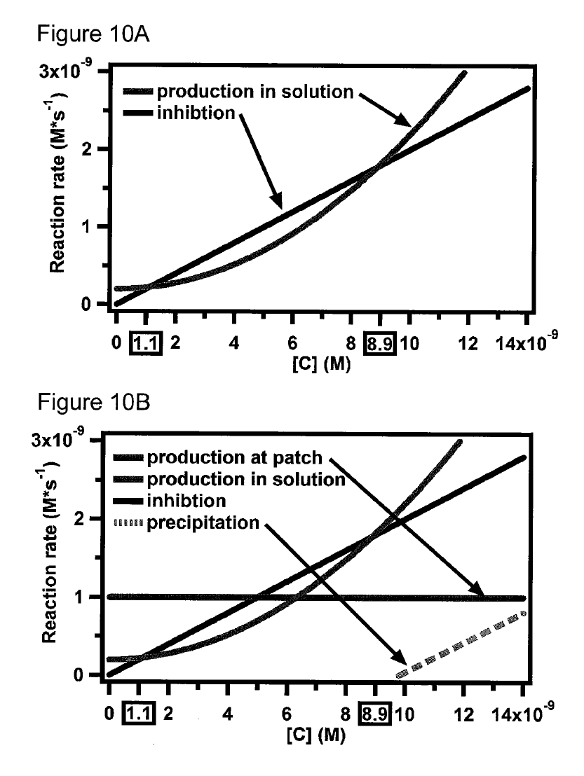

Three rate equations were incorporated into the simulation: i) production of C at the surface of the patch, rate=k_{patch,}; ii) autocatalytic production of C in solution, rate=k_{prod}[C]²+b; and iii) linear consumption of C in solution, rate=−k_{consum}[C]. The values used were [C]ᵢₙᵢₜᵢₐₗ=1×10⁻⁹ M, k_{patch}=1×10⁻⁹ M s⁻¹, k_{prod}=2×10⁷ M⁻¹s⁻¹, b=2×10⁻¹⁰ M s⁻¹, and k_{comsum}=0.2 s⁻¹. These values were selected on the bases of approximate values for representative reactions in blood clotting (Kuharsky and Fogelson, 2001, Biophys. J. 80: 1050-1074). Using these values, two steady states were present, one at [C]=1.1×10⁻⁹ M, and one at 8.9×10⁻⁹ M. The existence of these steady states may be understood by considering the rate plots for the reaction rate equations (FIG. 10). For a review describing rate plots, see Tyson et al., 2003, Curr. Opin. Cell Biol. 15: 221-231.

FIG. 10 illustrates how rate plots of the rate equations are incorporated in the numerical simulation of the modular mechanism (see text above for details). FIG. 10A shows two rate equations representing i) the module of autocatalytic production of C (curved line), and ii) the module of the linear consumption of C (straight line). The crossing points between these two lines represent steady states. The steady state at [C]=1.1×10⁻⁹ M is stable. However, the steady state at [C]=8.9×10⁻⁹ M is unstable and represents Cₜₕᵣₑₛₕ, the threshold [C]. When [C]>Cₜₕᵣₑₛₕ, the rate of production is greater than the rate of consumption and rapid amplification of [C] occurs. FIG. 10B illustrates two additional equations representing i) the reactions involved in production of C at the surface of the patch (horizontal line), and ii) the module of precipitation that occurs at high [C] (dashed line). The precipitation module was not incorporated in the simulation (although it was incorporated in the experimental chemical model), and has been included here schematically for clarity.

The steady state at [C]=8.9×10⁻⁹ M was unstable and represented the threshold concentration of C, Cₜₕᵣₑₛₕ. When [C]>Cₜₕᵣₑₛₕ, rapid amplification occurred, which led to the production of sufficient [C] to initiate precipitation (formation of the solid “clot”). In simulations that did not have a patch (where the patch size, p, was zero), [C] remained at the stable steady state value of [C]=1.1×10⁻⁹ M. When a large patch was incorporated into the simulation the combined production of C in solution and on the patch resulted in [C] exceeding [C]ₜᵣ, in 10 s.

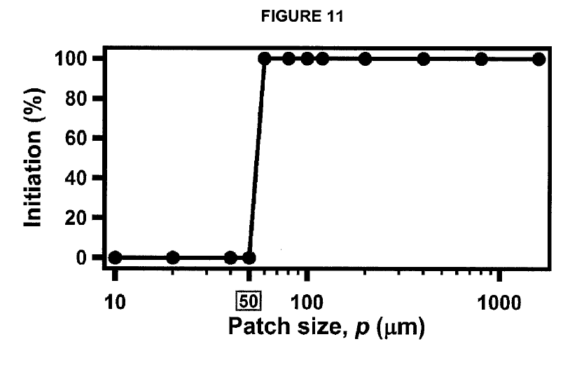

Results of the simulation. The concentration profiles obtained by numerical simulation indicated that “clotting” in the simulations displayed a threshold response to the patch size, p (FIG. 11). Using the parameters above, for patches p=50 μm, [C] never increased to Cₜₕᵣₑₛₕ. However, when p=100, [C] increased to Cₜₕᵣₑₛₕ in 10 s. The threshold patch size, pₜᵣ, (smallest p that will initiate clotting) was between 50 μm and 60 μm. The value of pₜᵣ increased as k_{patch} was decreased, indicating that the rate of production at the surface of the patch will affect pₜᵣ. This change in pₜᵣ, due to the change in rate of production at the surface of the patch, is consistent with preliminary experimental results that showed that when the TF concentration was decreased, t_{R} increased, and pₜᵣ increased. In the numerical simulations, the value of pₜᵣ also increased as D was increased.

FIG. 11 illustrates how the numerical simulation indicated that the probability of initiating “clotting” in the model exhibits a threshold response to patch size. In simulation, patches p≦50 μm never initiated “clotting”, but patches p≦60 μm always initiated “clotting”.

The quantitative agreement of the simulation with the experiment may be coincidental. The timescale of reaction, t_{R}, a single experimentally determined parameter, is a simpler and more reliable predictor of the size of the threshold patch for different blood plasma samples.

Numerical simulation for “clotting” on tight clusters of sub-threshold patches. The effect of changing the distance between sub-threshold patches on the concentration profile of C and on “clot time” was determined. A cluster of sub-threshold patches p=40 μm, generated [C]>Cₜₕᵣₑₛₕ only when positioned sufficiently close together: when 40 μm patches were separated by 80 μm, Cₜₕᵣₑₛₕ was never reached, however if patches were separated by only 20 μm, Cₜₕᵣₑₛₕ was rapidly reached and “clotting” initiated.

Preparing the PDMS Microfluidic Chamber for Experiments with Blood Plasma

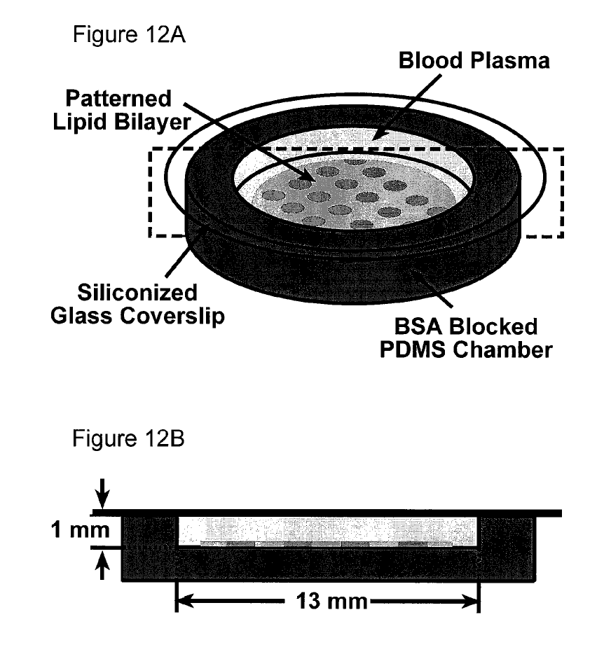

Designing and fabricating the chamber. The microfluidic chambers (FIG. 12) used in the blood plasma and whole blood experiments were constructed primarily from poly(dimethylsiloxane) (PDMS), fabricated from multi-level, machine-milled, brass masters. The disposable PDMS chamber had an inner diameter of 13 mm, an outer diameter of 20 mm, and a depth of 1 mm.

FIG. 12 illustrates the experimental set-up for experiments with blood plasma and patterned phospholipid bilayer substrates. FIG. 12A is a schematic of a PDMS microfluidic chamber (gray) used to contain a glass coverslip coated with a patterned phospholipid bilayer. Clot-promoting negatively charged phospholipids with reconstituted tissue factor (TF) (dark gray circles) were patterned in a background of inert neutral lipids. The chamber contained blood plasma, and was sealed closed with a siliconized glass coverslip on top. FIG. 12B is a cross-section of the chamber.

Eliminating convective flow, and background clotting in the chamber. To reduce convective flow in the solution, the PDMS chamber was soaked in a solution of NaCl (150 mM) for 4-8 hrs. To further reduce convective flow and reduce background clotting on the PDMS surface, the chamber was then soaked in a 1% BSA (in phosphate buffered saline (PBS) solution pH=7.3) for 1-2 hrs. Prior to the blood plasma or whole blood experiment, the chamber was rinsed thoroughly with a solution of NaCl (150 mM). To allow a good seal to form between the PDMS and the siliconized glass coverslip, a portion of BSA was removed from the top outer surface of the chamber by wiping with a dust free wipe.

Assembling the chamber for clotting experiments. The soaked chamber was placed in a 35×10 mm petri dish (BD Biosciences). The substrate (patterned coverslip) was placed in the chamber. A thin layer of Krytox fluorinated grease was applied on top of the chamber. The appropriate blood plasma or whole blood sample (see below) was then placed in the chamber. A siliconized glass coverslip was then pressed down lightly, pushing out excess blood plasma, making contact with the grease, and sealing the chamber. The petri dish was then filled with a solution of NaCl (150 mM), keeping the chamber submerged to eliminate evaporation through the PDMS. The chamber was maintained at either 23-24° C. or 37° C.

Measuring convective flow inside the chamber. In control experiments, the flow inside the PDMS chamber was measured by taking time-lapse fluorescent micrographs of fluorescent microspheres (FluoSpheres) in normal pooled blood plasma. The distances traveled by individual FluoSpheres were measured and divided by the elapsed time. The stock solution of FluoSpheres (sulfate microspheres, 1.0 μm diameter, yellow-green fluorescent (505/515), 2% solids) was diluted (25 μL to 5 mL) with a solution of NaCl (150 mM). The diluted FluoSphere solution was vortexed for 30 s and sonicated for 1 min to break up aggregates of FluoSpheres. This FluoSphere solution (70 μL) was added to citrated normal pooled blood plasma (210 μL). The FluoSphere/plasma mixture was added to the chamber and the chamber was sealed. Images were taken every 1 min at up to 10 positions throughout the chamber.

### Preparing Patterned Supported Phospholipid Bilayers to Spatially Control the Initiation of Clotting Via the Tissue Factor (TF) Pathway

Cleaning coverslips to reduce contamination and to generate a hydrophilic surface. To obtain reproducible results in clotting experiments with phospholipid bilayers, it was essential to eliminate contaminants such as large glass particles and dust. The cleaning process of coverslips consisted of the following steps: 1) applying 3M Scotch tape (#810) to remove large glass particles, 2) sonicating using the solution cycle (i. EtOH, ii. H₂O, iii. 10% ES 7× detergent, iv. EtOH, v. Millipore filtered water) with H₂O and EtOH rinses between steps to further eliminate loose glass particles, 3) soaking in a freshly made “piranha” solution (H₂SO₄:H₂O₂, 3:1, by volume; this mixture reacts violently with organic materials and must be handled with care) for approximately 20 min, and 4) rinsing thoroughly with Millipore filtered water and drying in a stream of N₂. The cleaned coverslips were used immediately after drying.

Preparing solutions of lipid-vesicles. The preparation of unilamellar vesicles has been described elsewhere (Yee et al., 2004, J. Am. Chem. Soc. 126: 13962-13972). Briefly, in a piranha cleaned glass vial, the appropriate chloroform solutions of lipids were mixed to the desired concentration and mole ratios. The chloroform was evaporated with a stream of N₂ (gas) and then the lipid cake was dried under vacuum (50 millitorr) for at least three hours. The dry lipids were suspended in Millipore filtered water (10 mg/mL) by vortexing and then hydrated overnight at 4° C. The hydrated vesicles were subjected to five freeze-thaw cycles. They were frozen in a dry ice/acetone bath and thawed in an oven set al a temperature above the lipid transition temperature. These vesicles were extruded (Lipex™ Extruder, Northern Lipids) ten times through a Whatman Nuclepore Track-Etch membrane (100 nm pore size) at a temperature above the lipid transition temperature. The extruded vesicles were diluted to the stock concentration (5 mg/mL) using Millipore filtered water and stored at 4° C. All vesicle solutions were used within two weeks.

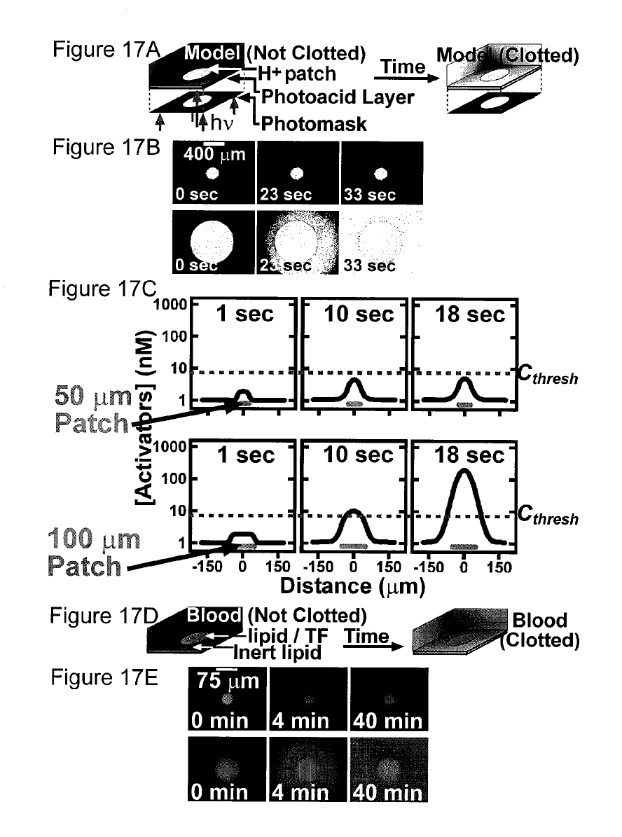

Reconstituting tissue factor (TF) to obtain clot-promoting vesicles. TF was reconstituted into mixed vesicles of DLPC/PS/Texas Reds DHPE (79.5/20/0.5 mole percents) at a concentration of 1.25 mg/mL in 1×HEPES-buffered saline/Ca²⁺ buffer. For experiments in FIGS. 17, 18, and 19 the TF concentration in the vesicle solution was 0.40 nM (TF:lipid ratio of 2.5×10⁻⁷). Assuming that all the TF was incorporated into the vesicles, the calculated surface concentration would be 0.08 fmol/cm². For experiments in Table 1, a final concentration of 0.16 nM of TF (TF:lipid ratio of 1×10⁻⁷) was used. After addition of TF to the vesicle solution, the solution was incubated at 37° C. for 30 min and then stored at 12° C. The vesicles were used within 18 hrs.

Forming an inert bilayer. The inert supported phospholipids bilayers consisted of DPPC (97%) and green fluorescent dye (3% of either Oregon Green® DHPE or NBD-DHPE) (Jung et al., 2005, Chem Phys Chem 6: 423-426). Bilayers were made by adding 215 μL of the DPPC vesicle solution (0.34 mg/mL vesicles in PBS) to a freshly cleaned coverslip in a hydrophilic PDMS chamber at 60° C. PDMS was made hydrophilic by oxidation with plasma cleaner (SPI Plasma Prep) prior to adding the coverslip. The microfluidic chamber containing the vesicle solution was incubated at 50° C. for 10 min and then cooled to room temperature. The excess vesicles were removed by repeated rinsing with a solution of NaCl (150 mM). The bilayers were stored in the dark at room temperature and used within 24 hr.

Backfilling into the inert bilayer to remove any areas of exposed glass. To ensure that there were no areas of exposed glass substrate caused by imperfections in the DPPC bilayers, all bilayers were backfilled with 30 μL of the DLPC vesicle solution (2.5 mg/mL vesicles in PBS buffer) and allowed to incubate in the dark at room temperature for 40 min. The excess vesicles were removed by extensive rinsing with a solution of NaCl (150 mM). These bilayers were photopatterned within a few hours.

Photopatterning to selectively remove patch regions of the inert bilayer. The DPPC bilayers that had been backfilled with DLPC were photopatterned using previously published methods (Yee et al., 2004, J. Am. Chem. Soc. 126: 13962-13972; Yu et al., 2005, Adv. Mater. 17: 1477-1480). Briefly, the bilayer coated coverslip was positioned on an aluminum alignment tray under a photomask (chrome on quartz, Photo Sciences, Inc.). This set-up was placed on a chilling plate (Echo Therm™, Torrey Pines Scientific) set to 0° C. to maintain a temperature of the sample at 20-30° C. during irradiation. Bilayers were irradiated for 7 min with deep UV light (Hanovia medium pressure 450 W Hg immersion lamp in a double walled cooled quartz immersion well) and then rinsed thoroughly with a solution of NaCl (150 mM). Patterned bilayers were backfilled within 2 hrs.

Generating patches by backfilling clot-promoting lipids into the photo-removed regions of bilayer. To generate the clot-promoting patches, the patterned bilayers were backfilled with 30 μL of the TF-reconstituted vesicle solution (1.25 mg/mL vesicles in PBS buffer) and allowed to incubate for 4 min at room temperature. Phospholipid bilayers containing active TF have been prepared previously (Contino et al., 1994, Biophys. J. 67: 1113-1116 (1994)). Excess vesicles were removed by vigorous rinsing with a solution of NaCl (150 mM). Patterned bilayers were used immediately in clotting experiments.

### Preparing Patterned Hydrophilic Patches on Silanized Glass Coverslips to Spatially Control the Initiation of Clotting Via the Factor XII Pathway

Forming an inert silanized surface on glass coverslips. A detailed procedure for silanization of glass coverslips has been previously described (Howland et al., 2005, J. Am. Chem. Soc. 127: 6752-6765). Briefly, freshly piranha cleaned glass coverslips were placed in a clean glass dish. Anhydrous hexadecane (10 mL) and n-octadecyltrichlorosilane (OTS) (40 μL) were added to the coverslips in a N₂(g) environment. This solution was incubated for 30 min. Then, a second 40 μL aliquot of OTS was added to the solution and was incubated for an additional 45 min. Excess OTS was removed by rinsing six times with anhydrous hexadecane followed by several rinses with EtOH. The silanized coverslips were stored under vacuum and used within 48 hrs.

Photopatterning to selectively generate hydrophilic glass patches in the inert silanized layer. Hydrophilic patches were generated using the photopatterning set-up described above and in the literature (Howland et al., 2005, J. Am. Chem. Soc. 127: 6752-6765). The silanized coverslips were irradiated under a photomask for 2 hrs. After irradiation, the coverslips were rinsed with EtOH and Millipore filtered water. The patterned coverslips were used with 30 min.

Detecting hydrophilic patches using a wetting test. Hydrophilic regions were detected using a glycerol wetting test (Wu and Whitesides, 2002, J. Micromech. Microeng. 12: 747-758). The patterned coverslips were coated with glycerol and the excess glycerol was removed using gentle vacuum. This process left droplets of glycerol only on the areas of the coverslip that were exposed to UV light (hydrophilic regions). After imaging, and prior to addition of normal pooled plasma, the glycerol was removed by vigorous rinsing with a solution of NaCl (150 mM).

### Preparing Human Blood Samples for Experiments

Preparing whole blood and platelet rich plasma from donor blood. Blood samples were obtained from individual healthy donors in accordance with the guidelines set by the Institutional Review Board (protocol #12502A) at The University of Chicago. Whole blood was collected in Vacutainer® tubes containing 3.2% sodium citrate (9:1 by volume). Platelet rich plasma (PRP) was obtained by centrifugation at 300×g for 10 min.

Preparing normal pooled plasma. Citrated normal pooled plasma (NPP) (human) (Butenas et al., Blood 105: 2764-2770) was purchased from George King Bio-Medical, Inc., and was stored in 1 mL aliquots at −80° C. until needed. When needed, the plasma was thawed by incubating at 18° C.

Recalcifying the blood plasma samples and adding the thrombi-sensitive dye. All blood plasma samples were recalcified by adding a solution of CaCl₂ containing the thrombin-sensitive fluorescent dye, Boc-Asp(OBzl)-Pro-Arg-MCA, (CaCl₂, 40 mM; NaCl, 90 mM; and Boc-Asp(OBzl)-Pro-Arg-MCA, 0.4 mM). At the start of each experiment, the plasma and the solution containing CaCl₂ were mixed 3:1 by volume. This recalcified plasma solution (400 μL) was added with gentle mixing to the experimental set-up shown in FIG. 9. Clotting was detected by the appearance of fibrin using bright field microscopy, and by the appearance of fluorescence signal generated when 4-Methyl-Coumaryl-7-Amine (MCA) was cleaved from Boc-Asp(OBzl)-Pro-Arg-MCA by thrombin.

Recalcifying the whole blood samples and adding the thrombin-sensitive dye. Whole blood samples were recalcified (Rivard et al., 2005, J. Thrombosis and Heamostasis 3, 2039-2043) by 1) first, mixing the whole blood (376 μL) with a thrombin-sensitive fluorescent dye, rhodamine 110-bis-(p-tosyl-L-glycyl-L-prolyl-L-arginine amide) (2 μL, 10 mM in DMSO) 2) then, the whole blood was mixed with a solution of CaCl₂ (23.5 μL, 200 mM). This recalcified whole blood solution was added to the experimental set-up shown in FIG. 12. Clotting was detected by the appearance of fluorescence signal generated when rhodamine 110 was cleaved from rhodamine 110-bis-(p-tosyl-L-glycyl-L-prolyl-L-arginine amide) by thrombin. The Rhodamine 110 dye was used for thrombin detection in the whole blood experiments, instead of the MCA dye, because red blood cells have a lower absorbance coefficient at the maximum excitation and emission wavelengths of rhodamine 110 than for MCA.

Inhibiting the factor XII pathway with corn trypsin inhibitor. For experiments measuring clot times for the TF pathway (all experiments using phospholipid bilayers and reconstituted TF), the factor XII (contact) pathway was inhibited with corn trypsin inhibitor (CTI). A stock solution of CTI (6.27 mg/mL) was added to the blood plasma, either immediately after the plasma was thawed (for NPP), or after centrifugation (for PRP), to a final concentration of 100 μg/mL, and incubated for approximately 10 hr at 18° C. prior to each experiment. For whole blood, CTI was added to a final concentration of 100 μg/mL after collection. For experiments measuring clot times for the factor XII (contact) pathway (all experiments with hydrophilic glass patches or gelatin), CTI was not added. Instead, the NPP was thawed and stored at 18° C. for 4 hr prior to each experiment.

### Imaging the Initiation of Clotting of Blood Plasma

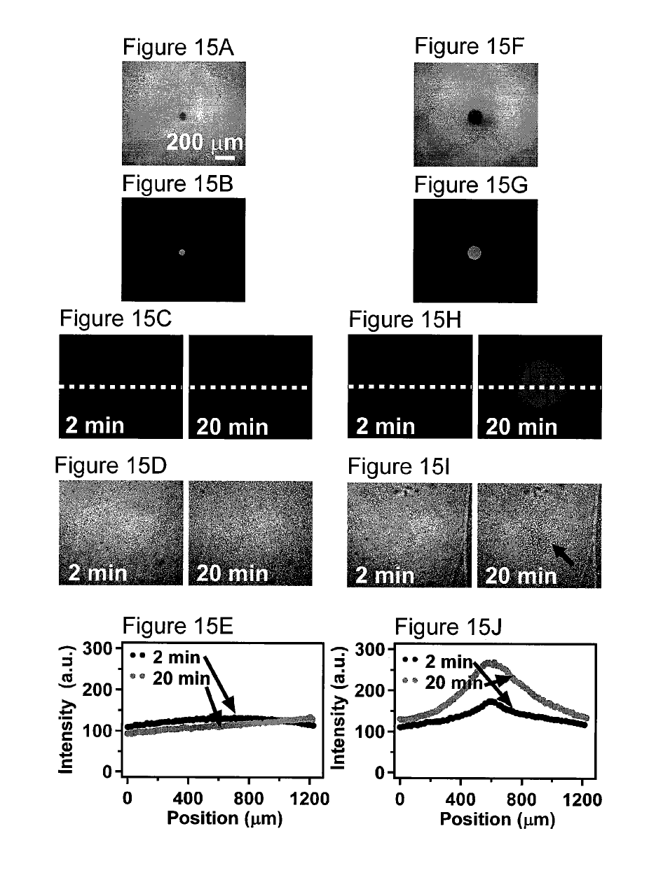

Detecting clotting and fluorescent lipids with fluorescence microscopy. Images were acquired using a Leica DMI 6000B epi-fluorescence microscope with a 10×0.4 NA objective coupled to a cooled CCD camera ORCA ERG 1394 (12-bit, 1344×1024 resolution) (Hamamatsu Photonics, K.K.) with a 0.65× coupler. Lighting was provided by a 75 W Xe light source. Three filter cubes were used: 1) DAPI/Hoechst/AMCA (λₑₓ=320-400 nm, λₑₘ=435-495) (chroma #31000v2) to detect MCA, 2) Texas Red (λₑₓ=530-590 nm, λₑₘ=600-680) (chroma #41004) to detect the Texas Red DHPE lipid dye, and 3) FITC/Bodipy/Fluo3/DiO (═ₑₓ=455-505 nm, λₑₘ=510-565) (chroma #41001) to detect the Oregon Green DHPE lipid dye, NBD-DHPE lipid dye, and rhodamine 110. Bright field microscopy (illumination from halogen lamp) was also used to detect formation of fibrin during clotting (see FIG. 15 for an example). MetaMorph® Imaging System (Universal Imaging Corp) was used to collect images. Images were processed using MetaMorph® Imaging System and Adobe Photoshop. All image adjustments were applied uniformly to the entire image, and to all sets of acquired images.

Analyzing images of initiation of clotting. The original grayscale fluorescence images of clotting and the phospholipid bilayers were false colored in MetaMorph®. The color was set by the emission wavelength of the filter cube. For all fluorescence images of clotting, the levels were adjusted to the same values. These images were copied and pasted directly from MetaMorph® into a new Adobe Photoshop document set to RGB mode. In Adobe Photoshop, the blue fluorescence images from MCA and representative red fluorescence images of the lipid bilayers were overlaid by screening the red images. All transformations were applied uniformly to every image, and all images were processed in an identical fashion.

Additional Control Experiments to Establish that Initiation of “Clotting” in the Chemical Model was Due to Photo-Induced Acid Generation at the Patch Only

Ruling out heating and photochemistry as sources of initiation of model reaction mixture. To minimize heating of the photomask, short-pass and IR filters were used to remove light with λ<300 nm and λ>400 nm. Irradiation of a photomask with no open patches did not initiate the reaction, indicating that the reaction is not triggered by heating of the mask. Irradiation in the absence of 2-nitrobenzaldehyde did not initiate the reaction, indicating that photochemistry of the chemical model itself does not induce initiation under the conditions used. In the absence of irradiation, the model reaction mixture was also stable for 500 to 1200 s.

Establishing that acid generation is dependent on patch area. To measure the amount of acid produced by the acidic patches (H⁺ production), the model system was replaced by a solution of an acid sensitive fluorescent dye, 5-(and 6)-carboxy-seminaphthofluorescein-1 (SNAFL) (see above for preparation of this solution). The H⁺ production was measured for various arrays of acidic patches by measuring the fluorescence intensity of SNAFL (FIG. 13). The H⁺ production was measured to establish that different arrays with the same total surface area, a, of acidic patches, but different sizes of individual patches, p, produced approximately the same amount of acid. Each array had the same total surface area of patches (a=5.03×10⁵ μm²), and each array produced approximately the same amount of acid (within a factor of two). A single 800 μm patch (a=5.03×10⁵ μm²) produced H⁺ at a rate of 2.9×10⁻² nmol/s, an array of 4×400 μm patches (a=5.03×10⁵ μm²) produced 3.4×10⁻² nmol/s, an array of 16×200 μm patches (a=5.03×10⁵ μm²) produced 2.6×10⁻² nmol/s, and an array of 64×100 μm patches (a=5.03×10⁵ μm²) produced 1.7×10⁻² nmol/s. A single 400 μm patch (a=1.26×10⁵ μm²) produced 7×10⁻³ nmol/s.

FIG. 13 illustrates how the amount of acid generated is dependent on the total surface area of the patches. In absence of the model reaction mixture, the H⁺ production was monitored with an acid sensitive dye, 5-(and-6)-carboxy-seminaphthofluorescein-1 (SNAFL, a dye with dual emission, dual excitation properties). First, a calibration curve of fluorescence intensity vs. H⁺ concentration was determined for SNAFL, by titration with HCl (data not shown). Then, the change in green and red fluorescence intensity of SNAFL was measured every 2 min following a 20 s pulse of UV light through the photomask and photoacid layer. Using the fluorescence intensity data, the measured calibration curve, and the known volume of the sample, the amount of H⁺ produced was determined. The H⁺ production was measured for different arrays of patches with the same total surface area, a, of patches, but different patch sizes, p. The H⁺ production was approximately the same for arrays with the same total surface area (within a factor of two). The H⁺ production was also measured for a single 400 μm patch, which had a surface area four times smaller than the arrays, and produced 2.4-4.8 times less H⁺.

The rates were determined by measuring the slopes of the H⁺ production lines (FIG. 13). The single 400 μm patch had four times smaller area than the p≦200 arrays, and produced approximately four times less acid, but was able to initiate “clotting” of the chemical model. The arrays of patches p≦200 did not initiate “clotting”. These results support the argument that the threshold was determined not simply by the total amount of acid produced, but by the size of the patch producing acid.

Quantifying the Fluorescence Intensity Profile of a pH-Sensitive Dye in the Chemical Model on the Photoacid Surface

Initiation of “clotting” in the chemical model caused a change from basic to acidic conditions and the quenching of red fluorescence from the dye bromophenol blue. For the model reaction mixture, the original grayscale time-lapse fluorescence images showed quenching of fluorescence (a shift from high fluorescence to low fluorescence) when “clotting” was initiated. In FIGS. 17 to 19, images of the chemical model were uniformly false-colored yellow and thresholded for dark objects. This procedure resulted in an inversion of light yellow and dark areas in all images.

FIG. 14 illustrates the quantification of fluorescence intensity profile of pH-sensitive dye in the chemical model on the photoacid surface. The fluorescence intensity of the original (unmodified) images was quantified to determine “clot” time in all experiments with the chemical model. FIG. 14A is a time-lapse fluorescent micrographs and linescans (dashed lines) of initiation of “clotting” in the chemical model on a 400 μm patch. Linescans show that at 22 sec “clotting” was initiated, and quenched the fluorescence. FIG. 14B shows time-lapse fluorescent micrographs and linescans of the chemical model on an array of 200 μm patches. Linescans show that “clotting” did not initiate on these patches, as the fluorescence intensity did not significantly decreases. Modifications and false-coloring of images did not distort the information, and analysis of false-colored images gave analogous intensity profiles.

When “clotting” was initiated there was a dramatic decrease in fluorescence intensity. A single 400 μm patch initiated “clotting” in 22 sec (FIG. 14A). The “clot” propagated away from the patch as a reactive front, quenching the fluorescence as it propagated. An array of 200 μm patches did not initiate “clotting” within 220 sec (FIG. 14B). The increased intensity at the patch was due to a small amount of red and green light passing through the clear patch of photomask from the light source above (see schematic of model system in FIG. 9). The fluorescence intensity appeared lower at the edges of the images due to normal non-uniform illumination at the low magnification used to measure fluorescence. In contrast, UV illumination from the top of the sample was defocused to yield a uniform illumination area of about 6 mm in diameter. As a control experiment, a uniform solution of a fluorescent dye was imaged, and it showed the same degree of non-uniformity and decreased intensity at the edges.

### Quantifying the Fluorescence Intensity Profile of a Thrombin-Sensitive Dye in Blood Plasma on Patterned Supported Phospholipid Bilayers

Initiation of clotting of blood plasma results in a burst of thrombin generation, accompanied by the onset of the formation of fibrin. To detect the initiation of clotting in blood plasma, fluorescence microscopy was used to detect the thrombin-induced cleavage of a peptide-modified coumarin dye, which releases 4-methyl-coumaryl-7-amide (MCA, blue fluorescence) (FIG. 15H), and brightfield microscopy to detect the formation of fibrin (FIG. 15I). For a 61 μm patch (FIG. 15 A to E) clotting of platelet rich plasma (PRP) did not initiate on the patch within 45 min.

FIG. 15 illustrates the quantification of initiation of clotting of blood plasma. Shown in FIGS. 15A and B is a 61 μm patch of TF-reconstituted bilayer containing a red lipid dye that was patterned in a background of an inert bilayer containing a green lipid dye. FIGS. 15C and D shows that no large increase in fluorescence intensity due to MCA was observed within 20 min on the 61 μm patch. No formation of cross-linked fibrin strands or platelet aggregation was observed on the 61 μm patch. FIG. 15E shows linescans (dashed lines in (C)) quantifying the fluorescence intensity in FIG. 15C. Shows in FIGS. 15F and G is a 137 μm patch of TF-reconstituted bilayer containing a red lipid dye that was patterned in a background of an inert bilayer containing a green lipid dye. Shown in FIGS. 15H and I is a large increase in fluorescence intensity due to release of MCA by thrombin was seen within 2 min on the 137 μm patch. Formation of crosslinked fibrin strands, and aggregation of platelets (solid white arrow), was observed on the 137 μm patch. The open white arrows point to imperfections in the PDMS chamber underneath the coverslip. FIG. 15J shows linescans (dashed lines in (H)) quantifying the fluorescence intensity in (H).

No large increase in fluorescence due to release of MCA by thrombin was observed (FIGS. 15 C and E), and no formation of cross-linked fibrin strands or aggregation of platelets was observed (FIG. 15D). This general response was seen for all patches that did not initiate clotting. For a 137 μm patch (FIG. 15 F to J), the clotting of PRP initiated on the patch within 2 min. A large increase in fluorescence due to release of MCA by thrombin was observed (FIGS. 15H and J). Both formation of cross-linked fibrin strands and aggregation of platelets were also observed (FIG. 15I). This general response was seen for all patches that initiated clotting.

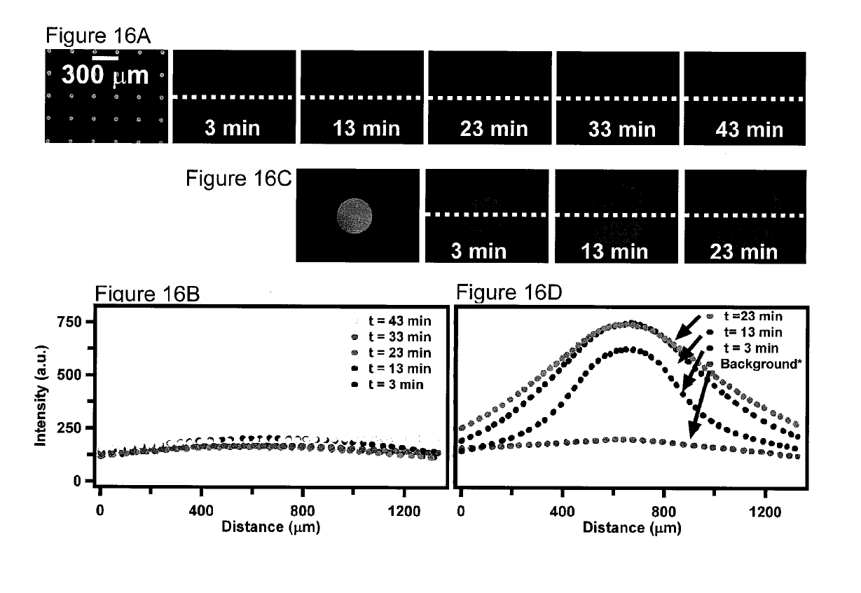

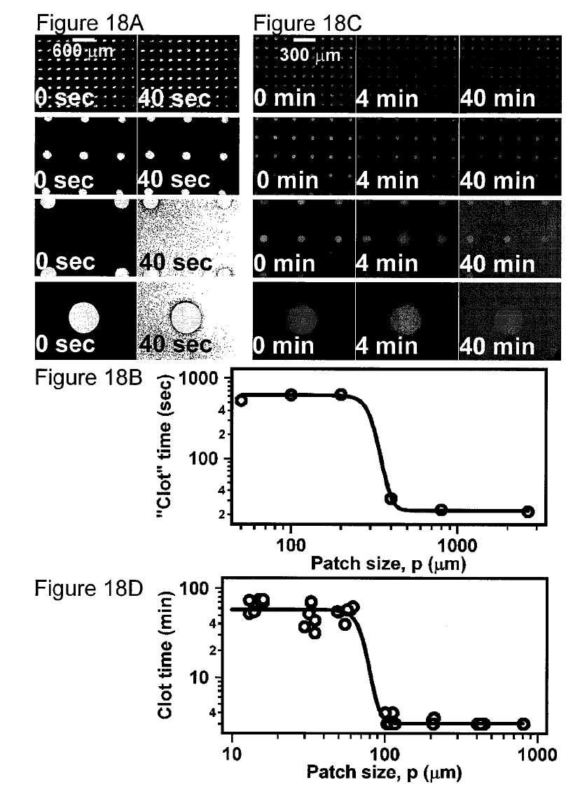

In the arrays of patches presented in FIGS. 18C and D, the same general responses were observed (FIG. 16). Shown in FIG. 16 is the quantification of initiation of clotting of blood plasma on arrays presented in FIG. 18D. FIGS. 16A and B shows how for arrays of 50 μm patches, clotting did not initiate on the patch within 43 min. No large increase in fluorescence due to release of MCA by thrombin was observed (FIGS. 16A and B), and no formation of cross-linked fibrin strands was observed. FIGS. 16C and D shows how for arrays of 400 μm patches, clotting initiated on the patches within 3 min. A large increase in fluorescence due to release of MCA by thrombin was observed (FIGS. 16C and D). Formation of cross-linked fibrin strands was also observed.

### Measuring and Eliminating Convective Flow in the Chamber Containing Blood Plasma

The flow inside the blood plasma chamber (FIG. 12) was measured by taking time-lapse fluorescent micrographs of fluorescent microspheres (FluoSpheres) in normal pooled blood plasma. The distances traveled by individual FluoSpheres were measured and divided by the elapsed time (see above for preparation of this solution). After the chamber was optimized to eliminate flow, the flow rate was typically less than 3 μm/min at 10 μm above the substrate, and less than 10 μm/min at 100 μm above the substrate. A flow rate of 3 μm/min is ten times smaller than the rate of spreading of initiated clotting (25-35 μm/min).

Steps taken to eliminate flow. The steps taken to eliminate flow included: i) using a sealed PDMS chamber to eliminate convective flow generated at the air/plasma interface (Marangoni flow) and evaporation, it) the PDMS chamber was soaked in a solution of NaCl (150 mM) for 4-8 hr to eliminate evaporation through the PDMS, and to maintain a constant osmotic pressure, iii) the chamber was then soaked in a 1% BSA in PBS (pH=7.3) for 1 hr to eliminate Marangoni flow generated at the PDMS/plasma interface due to possible gradients in surface tension, iv) the chamber was submerged in a solution of NaCl (150 mM) after plasma was sealed inside, v) the amount of irradiation during microscopy was minimized, and vi) stage movement was minimized.

Comparing the Threshold of Donor Platelet Rich Plasma with Normal Pooled Plasma at 24° C. and 37° C.

The threshold patch size of donor platelet rich plasma and normal pooled plasma was measured at 24° C. and 37° C. Clot times were measured on patches presenting clotting stimuli (TF-reconstituted bilayers) in arrays containing patches of different sizes (Table 1). In a single experiment, the clot time on seven different patch sizes was measured. The concentration of TF in vesicles used to prepare the bilayers in Table 1 was 0.16 nM (TF:lipid ratio of 1×10⁻⁷). This value is a factor of 2.5 less concentrated than that used in the experiments described in the main text (0.40 nM). For normal pooled plasma (NPP), using [TF]=0.16 nM yielded a longer timescale of reaction, t_{R}=206 s, than using [TF]=0.40 nM (t_{R}=30 s), and a corresponding larger threshold patch size, pₜᵣ[m] (160±32 μm for [TF]=0.16 nM vs. 75±25 μm [TF]=0.40 nM). Clot time vs. patch size for platelet rich plasma (PRP) from donors was measured. For a given [TF], PRP had a shorter t_{R} (40 s for donor X, and 48 s for donor Y) than NPP (206 s) and a corresponding smaller pₜᵣ (85±26 and 90±7 μm for PRP vs. 160±32 for NPP).

TABLE 1
Threshold patch size, pₜᵣ, and timescale of reaction, t_{R},
for PRP and NPP at 24° C. and 37° C.

| Blood | | | | | |
|---|---|---|---|---|---|
| Sample | Temp (° C.) | t_{R} (s) | t_{R}^{1/2} (s^{1/2}) | Pₜᵣ ± σ(μm)* | Blood Source |
| PRP | 24 | 40 | 6.3 | 85 ± 26 | Donor X |
| PRP | 24 | 48 | 6.9 | 90 ± 7 | Donor Y |
| NPP | 24 | 206 | 14.4 | 160 ± 32 | G. King, Inc |
| PRP | 37 | 26 | 5.1 | 90 ± 15 | Donor Y |
| NPP | 37 | 121 | 11.0 | 125 ± 15 | G. King, Inc |

*The value of pₜᵣ was determined by averaging the pₜᵣ obtained from each array (3-6 arrays total per blood sample). In each array seven different patch sizes were measured. σ is the standard deviations for values of pₜᵣ.

### Modular Chemical Mechanism Predicts Initiation in Hemostasis

The inventors demonstrated that a simple chemical model system, built using a modular approach, may be used to predict the spatiotemporal dynamics of initiation of blood clotting in the complex network of hemostasis. Microfluidics was used to create in vitro environments that expose both the complex network and the model system with surfaces patterned with patches presenting clotting stimuli. Both systems displayed a threshold response, with clotting initiating only on isolated patches larger than a threshold size. The magnitude of the threshold patch size for both systems was described by the Damköhler number, measuring competition of reaction and diffusion. Reaction produces activators at the patch, and diffusion removes activators from the patch. The chemical model made additional predictions that were validated using human blood plasma, suggesting that such chemical model systems, implemented with microfluidics, may be used to predict spatiotemporal dynamics of complex biochemical networks.

To model the spatiotemporal dynamics of the initiation, the approximately 80 reactions of hemostasis were represented as three interacting modules, with the overall kinetics corresponding to i) higher-order autocatalytic production of activators, ii) linear consumption of activators, and iii) formation of the clot at high concentrations of activators. Concentration of activators, C, acted as a control parameter. Interactions among these modules lead to a threshold concentration, Cₜₕᵣₑₛₕ, above (but not below) which clotting was initiated. In this representation, hemostasis is normally in the stable steady state at low C. Small increases of C preserve C<Cₜₕᵣₑₛₕ, such perturbations decay, and the system returns to the stable steady state. Large perturbations increase the concentration above the unstable steady state (C>Cₜₕᵣₑₛₕ), and result in amplification of activators leading to initiation of clotting. Thus, a functional, but drastically simplified, chemical model of hemostasis may be created by replacing each module with at least one chemical reaction with kinetics matching that of the module.

FIG. 17 illustrates how human blood plasma and the simple chemical model both initiate clotting with a threshold response to the size of patches presenting clotting stimuli. FIG. 17A is a simplified schematic of a microfluidic device used to test threshold response in initiation of “clotting” in the chemical model. The reaction mixture was kept over a photoacid surface containing 2-nitrobenzaldehyde. UV-irradiation through a photomask photoisomerized 2-nitrobenzaldehyde (not acidic) to 2-nitrosobenzoic acid (acidic, pKa<4) creating acidic patches of “clotting” stimuli (green). When “clotting” was initiated, the basic reaction mixture became acidic, and turned yellow.

FIG. 17B shows time-lapse fluorescent micrographs of initiation of “clotting” (false-colored yellow) in the chemical model on patches p=200 μm (top, no initiation) and p=800 μm (bottom, rapid initiation). FIG. 17C shows numerical simulations qualitatively describing the competition between production of clotting activators at the patch, and diffusion of activators away from the patch, in regulating initiation of clotting. For sub-threshold patches (top, 50 μm) diffusion dominates, and the concentration of activators never reaches the threshold concentration Cₜₕᵣₑₛₕ (dashed line) necessary to initiate clotting. For above-threshold patches (bottom, 100 μm), the production of activators dominates, exceeding Cₜₕᵣₑₛₕ, leading to rapid amplification of activators and to clotting.

FIG. 17D is a schematic of an in vitro microfluidic system used to contain blood plasma and to expose it to patches presenting clotting stimuli. Patches of negatively charged phospholipid bilayers with reconstituted tissue factor (lipid/TF) (red fluorescence) were patterned in a background of inert lipids. Blue represents clotting. FIG. 17E shows time-lapse fluorescent micrographs of initiation of clotting (blue fluorescence) of blood plasma on red patches p=50 μm (top, no initiation) and p=100 μm (bottom, rapid initiation), where p[m] is the diameter of the patch.

### Initiation of “Clotting” in the Chemical Model Showed a Threshold Response to Patch Size

To observe the qualitative dynamics of this chemical model system, the inventors tested whether initiation of “clotting” on acidic patches was robust (initiating on large but not small patches) (FIG. 18A). UV light was used as a stimulus for initiating “clotting”. Photochemical production of acid was spatially confined to patches using a photomask. Acid diffused from the surface patch into the solution, and the “clotting” reaction was initiated only if the local concentration of acid exceeded the threshold value Cₜₕᵣₑₛₕ.

FIG. 18 illustrates how the chemical model correctly predicts that in vitro initiation of clotting in human blood plasma depends on the spatial distribution, rather than the total surface area of a lipid surface presenting tissue factor (TF), an activator of clotting. FIG. 18A is a time-lapse fluorescent micrographs of initiation of “clotting” (yellow) in the chemical model on arrays of patches p=50, 200, 400, and 800 μm (top to bottom, green). All arrays had the same total surface area of patches (5×10⁵ μm²). “Clotting” did not initiate on arrays of patches p=50-200 μm, but rapidly initiated on patches p=400-800 μm. FIG. 18B is a graph quantifying the threshold response for initiation of “clotting” in the chemical model, using data as shown in A. FIG. 18C is a time-lapse fluorescent micrographs showing initiation of clotting (blue) of blood plasma on arrays p=100 μm and p=400 μm patches (red), but no initiation on arrays of p=25 μm and p=50 μm patches (red). The total surface area of patches in all arrays was the same (3.5×10⁶ μm²). FIG. 18D is a graph quantifying the threshold response for initiation of clotting of blood plasma, using data as shown in C. Clot times were determined by monitoring the appearance of fibrin.

Initiation of “clotting” in the chemical model showed a threshold response to patch size, p[m], the diameter of a circular patch (FIGS. 18B, 17 experiments). Single patches p≧400≧pₜᵣ μm reliably initiated “clotting” in about 22 s, while single patches p≦200<pₜᵣ μm did not cause initiation within 500 s. Control experiments verified that initiation was due to the production of acid at the surface, and not due to heating of the sample or photochemistry of the solution.

Initiation of “Clotting” in the Chemical Model May be Described by the Damköhler Number

To obtain a semi-quantitative description of the dynamics in this system, the inventors estimated the threshold patch size, pₜᵣ[m], (size p of the smallest patch that initiates clotting) by considering competition of reaction and diffusion. Reaction produces an activator at the patch on the time scale t_{R} [s], and diffusive transport removes the activator from the patch on the time scale t_{D} [s]. For patches p<pₜᵣ diffusion dominates (t_{D}<t_{R}), and the concentration of activator never reaches the threshold Cₜₕᵣₑₛₕ. For patches p>pₜᵣ reaction dominates (t_{D}>t_{R}), local concentration of activator exceeds the threshold Cₜₕᵣₑₛₕ, and initiates “clotting”. This competition is described by the Damköhler number (Bird et al., 2002, Transport Phenomena, John Wiley & Sons, New York, 2^{nd} ed.), and pₜᵣ corresponds to p at which t_{R}≈t_{D} (FIG. 18C). Since t_{D}≈p²/D, pₜᵣ should scale as pₜᵣ (D×t_{R})^{1/2}, where D [m²s⁻¹] is the diffusion coefficient of the activator. This scaling prediction is reasonable, and consistent with the one originally proposed for membrane patch size regulating a proteolytic feedback loop on a membrane during clotting (Beltrami and Jesty, 2001, Math. Biosci. 172: 1-13). For the chemical model system, experimental value 200<pₜᵣ<400 μm agreed with predicted pₜᵣ about 470 μm, calculated using D(H⁺) about 10⁻⁸ m²s⁻¹, and t_{R} about 22 s.

### The Chemical Model Correctly Predicts the Spatiotemporal Dynamics for Initiation of Clotting

This chemical model makes four predictions for initiation of blood clotting. First, it predicts the existence and the value of the threshold patch size, pₜᵣ. To test this prediction, and to probe the dynamics of the initiation of the hemostasis network, the inventors developed an in vitro microfluidic system to control the initiation of clotting in space and time (FIG. 18D). Patterned supported phospholipid bilayers were used to present patches of the clotting stimulus, a lipid surface containing phosphatidylserine with reconstituted human tissue factor (TF), which was incorporated into bilayers. TF is an integral membrane protein that is exposed at sites of vascular damage and atherosclerotic plaque rupture. These clot-inducing patches were surrounded by background areas of inert lipid bilayers (phosphatidylcholine). A microfluidic chamber was used to contain freshly recalcified plasma over the patterned lipid surface, and to eliminate convection.

Initiation in the hemostasis network may occur through two pathways, the TF pathway, and the factor XII pathway. In experiments testing initiation by TF, corn trypsin inhibitor was used to inhibit the factor XII pathway. “Initiation” in this network refers to the clotting process that culminates in a spike of thrombin and the onset of formation of fibrin. Bright-field microscopy was used to detect formation of fibrin, and fluorescence microscopy to detect thrombin-induced cleavage of a peptide-modified coumarin dye. The clot times reported here indicate the time that fibrin appeared, and in all experiments appearance of fibrin correlated to the increased fluorescence. Fluorescence images of clotting were uniformly thresholded to reduce the background fluorescence of the dye.

Initiation of clotting of blood plasma in this microfluidic system displayed a threshold response to patch size. Patches p≧100 μm initiated clotting in less than three minutes (40 of 44 experiments), while patches p≦50 μm did not initiate clotting (28 of 28 experiments, at least thirty patches per experiment) (FIG. 18E). Background clotting was observed in 32-75 min in experiments with patches p≦50 (generally initiating not on the patches), consistent with 45-70 min range for initiation on surfaces that had no patches at all, and consistent with the background clotting times reported by others. Initiated clotting spread as a reactive front at 25-35 μm/min. To predict the value of pₜᵣ, D about 5×10⁻¹¹ m²s⁻¹ was used (approximate value for thrombin as a representative activating protein involved in the amplification of the clotting cascade), and t_{R} about 30±5 s was used (obtained by measuring the initiation time of clotting on a non-patterned clot-inducing bilayer). Predicted pₜᵣ about 40 μm agreed with the measurement 50<pₜᵣ<100 μm. A considerably smaller threshold patch size (few μm) was proposed previously by considering diffusion of an activator in a membrane. The results indicate that pₜᵣ is determined by diffusion of a protein in solution.

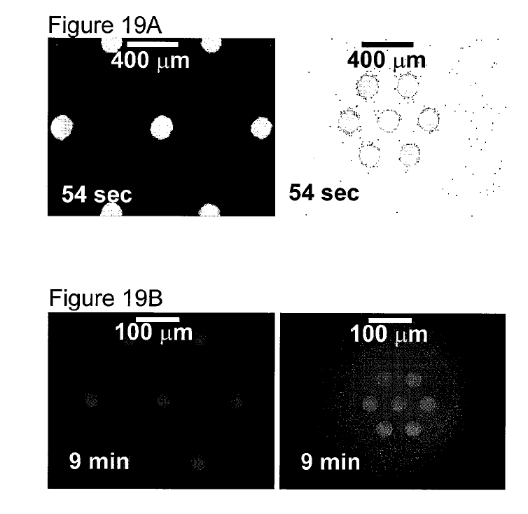

Second, the model predicts that the size of individual patches (isolated, non-interacting), rather than their total surface area, determines initiation of clotting. To demonstrate this effect, the chemical model was exposed to arrays of patches (FIGS. 19A and B).

FIG. 19 illustrates how the chemical model correctly predicts that initiation of clotting of human blood plasma can occur on tight clusters of sub-threshold patches that communicate by diffusion. FIG. 19A shows fixed-time (54 s) fluorescent micrographs of clusters of sub-threshold patches p=200 μm in the chemical model system. These patches initiated “clotting” when separated by 200 μm (right) but not 800 μm (left). FIG. 19B shows fixed-time (9 min) fluorescent micrographs of clusters of sub-threshold patches p=50 μm (red) exposed to blood plasma. These patches initiated clotting when separated by 50 μm (right) but not 200 μm (left).

Each array had the same total surface area of patches (5×10⁵ μm²), and produced the same amount of acid, but only arrays with patches p≧400 μm initiated “clotting”. Total area was irrelevant: a single above-threshold patch quickly initiated “clotting”, even though it had four times smaller area than an array of sub-threshold patches, and produced about four times less acid. Clotting of blood plasma (FIGS. 19C and D) also displayed this dynamics—among arrays of patches of the same total surface area, only arrays with patches p≧100 μm initiated clotting (six measurements per patch size). Initiation of clotting was exquisitely sensitive to the spatial distribution of TF in the sample. Knowing the amount of TF in the sample was not sufficient to predict whether initiation would occur—in the experiments with constant volumes of blood plasma, above-threshold patches induced clotting, while an array of sub-threshold patches with a total surface area 20 times larger, bearing 20 times more TF, did not.

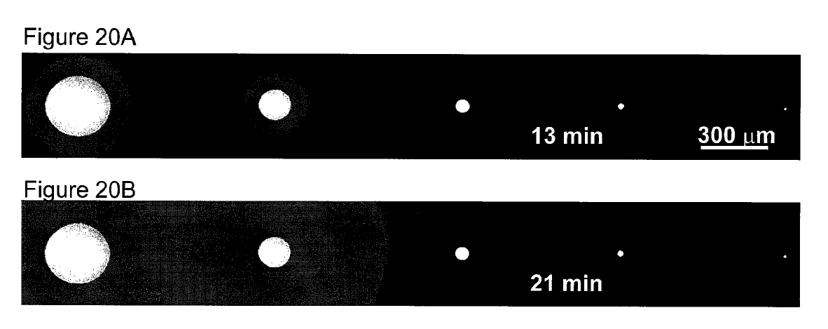

Third, the model predicts that a sufficiently tight cluster of sub-threshold patches should initiate clotting (FIG. 20). The images in FIG. 20 illustrate how the chemical model correctly predicts initiation of clotting via the second (factor XII) pathway, suggesting that the model describes the dynamics of initiation of the entire complex network of hemostasis in vitro. Test of initiation of clotting via the factor XII pathway in human blood plasma on glass is shown. Two time-lapse fluorescent micrographs 13 min (FIG. 20A) and 21 min (FIG. 20B) showing initiation of clotting on an array of clot-inducing hydrophilic glass patches p=400, 200, 100, 50, and 25 μm (left to right, white), patterned in a background of inert silanized glass. For the blood plasma sample shown here, the threshold patch size was between 100 μm and 200 μm.

Production of the activator on patches at the perimeter of the cluster reduces the diffusive flux of the activators away from the central patch. For a given t_{R}, initiation of clotting should occur for sub-threshold patches spaced closer than the diffusion length scale, equal to pₜᵣ. To demonstrate this effect, the inventors exposed the chemical model (200<pₜᵣ<400) to two clusters of sub-threshold patches (FIG. 20A). Clusters of 200 μm patches separated by 200 μm rapidly initiated “clotting”, while clusters separated by 800 μm did not. Numerical simulations agreed with these experiments. These predictions were verified with blood plasma (50<pₜᵣ<100), where clusters of 50 μm patches separated by 50 μm rapidly initiated clotting, while clusters of patches separated by 200 μm did not (nine experiments, FIG. 20B). It is known that amplification of activators could happen much more rapidly on the surfaces of membranes, especially of platelets, and these results further confirm the importance of transport in solution in setting pₜᵣ.

Fourth, if this chemical model represents the overall dynamics of initiation in the network, rather than a subset of reactions in the TF pathway, it suggests that initiation of blood clotting via the factor XII pathway would also show a threshold response. To initiate this pathway the inventors exposed blood plasma to negatively-charged glass; initiation occurred in t_{R} about 9 min. The inventors used the diffusion coefficient for thrombin to predict the threshold patch size pₜᵣ about (D×t_{R})^{1/2} about 160 μm. To test this prediction, patches of hydrophilic glass were created in a background of inert, hydrophobic silanized glass. pₜᵣ about 100 μm was rapidly determined by placing blood plasma on a single array of patches of different sizes (FIG. 9). In all 14 experiments, patches p≧200 μm induced clotting, but patches p<50 μm did not. Patches p=100 μm were close to threshold size, initiating clotting (12-19 min) in only four of fourteen experiments, consistent with either slight variations of the surface chemistry from patch to patch, or the stochastic nature of the initiation of clotting via the factor XII pathway. The ability of subject's blood to initiate clotting by either the TF or factor XII pathways can thus be rapidly evaluated by measuring the threshold response on a single slide with array of patches of different sizes.

Mechanism that Describes Clot Propagation in the Network of Hemostasis

One approach to understanding the regulatory mechanisms of hemostasis, as for any complex biochemical network, is to develop models of the network. FIG. 21 illustrates a simple chemical model that mimics the dynamics of hemostasis based on a simple regulatory mechanism—a threshold response caused by the competition between production and removal of activators. This threshold response is manifested by clotting occurring only when the concentration of activators, C_{act}, exceeds a critical concentration, C_{crit}. This mechanism made two predictions: 1) a clot propagates as a reactive front with a constant velocity, Fᵥ[m s⁻¹], if C_{act}, remains above C_{crit}, and 2) for a given geometry of vessels, clot propagation from an obstructed vessel into an unobstructed vessel with flowing blood is dependent on the shear rate, \dot{γ}[s⁻¹], in the vessel with flowing blood.

FIG. 21 is a schematic drawing of the proposed mechanism for regulation of clot propagation through a junction of two vessels at high (a) and low (b) shear rates. Clotting (blue) initiates when the concentration of activators (), C_{act}, exceeds a critical concentration, C_{crit}. This clot propagates through an obstructed vessel as a reactive front with a velocity, Fᵥ[m s⁻¹], when C_{act} remains above C_{crit}. When the propagating clot reaches a junction between two vessels (junction), propagation stops or continues depending on the shear rate, \dot{γ}[s⁻¹], in the vessel with flowing blood (flow vessel) at the junction. FIG. 21a illustrates how clot propagation stops at a junction when \dot{γ} in the flow vessel is above the threshold shear rate, \dot{γ}ₜₕᵣₑₛₕ, because activator in the flow vessel is removed from the growing clot faster than it is produced, maintaining C_{act} in the flow vessel below C_{crit}. FIG. 21b illustrates how clot propagation continues through the junction when r in the flow vessel is below \dot{γ}ₜₕᵣₑₛₕ, because activator in the flow vessel is removed from the growing clot slower than it is produced, causing C_{act} in the flow vessel to exceed C_{crit}.

This invention provides a microfluidic system that offers a compromise between in vivo and simple in vitro experiments. It allows precise control of flow, geometry, and surfaces. This system was used with human blood plasma to test the predictions of the proposed mechanism and demonstrated that this simple mechanism provides insight into the regulation of the spatiotemporal dynamics of clot propagation.

Clots Propagate as a Reactive Front with a Constant Velocity in the Absence of Flow

To test the prediction that clots propagate as a reactive front with a constant velocity, the inventors used a microfluidic system to regulate and observe clotting in human blood plasma. This system was fabricated in poly(dimethylsiloxane) (PDMS).

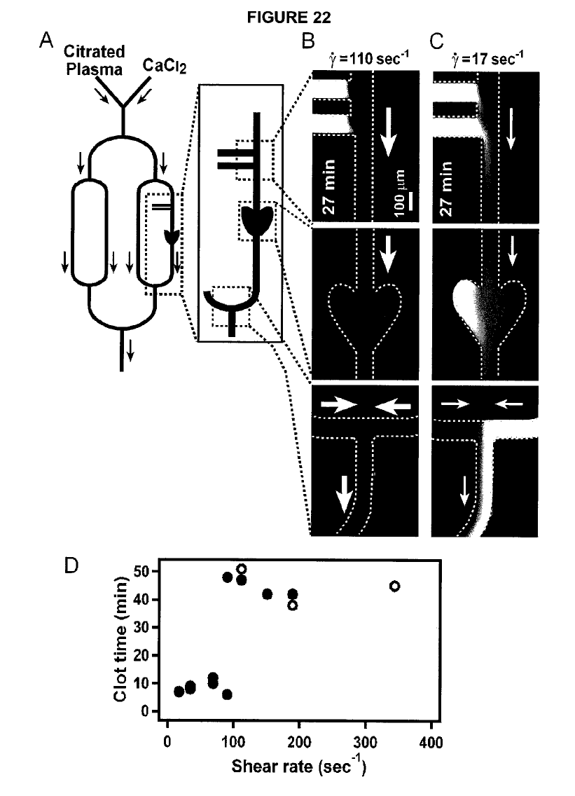

FIG. 22 illustrates measurement of the propagation of a blood clot through a microfluidic channel in the absence of flow. Clots propagate with a similar velocity, Fᵥ, in the absence and presence of a membrane-bound inhibitor of clotting, thrombomodulin (TM), on the channel wall. FIG. 22a is a schematic drawing of the procedure for initiating and monitoring clot propagation in a microfluidic device. Clotting initiated only on the lipid-TF-coated channel walls, not on the inert lipid, and propagated into the section of the device where inert lipids coated the channel walls. FIG. 22b is a fluorescence microphotograph of a microfluidic device showing that lipids with reconstituted TF (lipid-TF) can be localized to a specific section of a channel in a background of inert lipids. FIG. 22c is a time-lapse fluorescence microphotographs showing position of the clot at 0, 40, and 80 min after plasma was introduced into the channel. FIG. 22d shows experiments quantifying the velocity of clot propagation in the absence of TM (Fᵥ≈20 μm min⁻¹) and in the presence of TM (lipid:TM=7.6×10⁴, Fᵥ≈25 μm min⁻¹ and lipid:TM=7.6×10³, Fᵥ≈24 μm min⁻¹).

Clot initiation and propagation were spatially separated by patterning the walls of the same channel with different phospholipids (FIG. 22a). This patterning was accomplished by flowing two laminar streams containing phospholipid vesicles into the device from opposite ends of the channel. One stream contained a mixture of lipids that initiate clotting—phosphocholine, phosphatidylserine, and Texas Red® phosphoethanolamine with reconstituted Tissue Factor (lipid-TF, FIG. 22a)—and the other stream contained a lipid that does not initiate clotting—phosphatidylcholine (inert lipid, FIG. 22a). Next, the channels were rinsed with an aqueous solution of NaCl to remove excess lipid vesicles, leaving a coating of lipid-TF or inert lipids on the channel walls (FIGS. 22a, b). Then, blood plasma was flowed into the device, allowed to contact the lipid-TF, and flow was stopped. Clotting was monitored using bright-field microscopy to detect fibrin formation and fluorescence microscopy to detect thrombin-induced cleavage of a peptide-modified coumarin dye.

Clotting initiated only where the channel walls were coated with lipid-TF. This clot propagated into the section of the device coated with inert lipid (FIG. 22a). This clot propagated throughout the channel as a reactive front with a constant velocity, Fᵥ≈20 μm min⁻¹ (FIGS. 22c, d).

Thrombomodulin on Channel Walls does not Affect Clot Propagation

It has been proposed that clot propagation is regulated by thrombomodulin (TM), an inhibitor of clotting located at on the walls of vessels near sites of vascular damage. It has been shown that clot propagation is reduced when TM is homogenously mixed into blood plasma. To mimic the localization of TM on vessel walls, TM was incorporated at the channel walls and tested if this TM was sufficient to stop clot propagation. The inventors incorporated TM into the inert phospholipid surface by forming inert lipid vesicles with reconstituted TM (lipid:TM) and by using the procedure described above to coat the channel walls.

Control experiments verified TM activity on the channel walls was on the same order of magnitude as previously measured for a monolayer of endothelial cells. Measured TM activities are shown in Table 2, which illustrates the quantification of activated protein C (aPC) production from Egg PC lipid coated surfaces with reconstituted thrombomodulin (TM). Corresponding velocities of clot propagation are shown.

TABLE 2
Quantification of activated protein C (aPC) production from Egg
PC lipid coated surfaces with reconstituted thrombomodulin (TM)

| | | | Calculated | | Front |
|---|---|---|---|---|---|
| | | TM:Egg | Surface | aPC | Velocity |
| Sur- | Temp. | PC | Density | production | (μm |
| face | (° C.) | ratio | (pmol m⁻²) | (pmol min⁻¹ m⁻²) | min⁻¹) |
| PDMS | 25 | N/A | N/A | N/A | 20 |
| PDMS | 25 | 1:75600 | 4 | 10 | 24 |
| PDMS | 25 | 1:7560 | 40 | 5* | 25 |
| Glass | 37 | N/A | N/A | N/A | 41 |
| Glass | 37 | 1:75600 | 4 | 0.3 | ND |
| Glass | 37 | 1:7560 | 40 | 10 | 50 |

*Saturation in TM concentration may have been reached (Tseng et al., 2006, Biomaterials 27: 2768-2775).
N/A = Not applicable because no TM was present. Data is shown only for the comparison of front velocities.
ND = Not determined.

When the mole ratio of lipid:TM was 7.6×10⁴, clots propagated at approximately the same velocity as without TM (Fᵥ≈25 μm min⁻¹, green triangles, FIG. 22c). To further show that TM located at the channel wall does not stop clot propagation, the TM density was increased by a factor of ten, and no appreciable change in Fᵥ was observed (FIG. 22c). Additional control experiments (see Table 2) showed a similar TM activity for both concentrations used here which is consistent with the saturation effects previously observed for high TM concentrations. Clot propagation in the presence of TM in this device (surface-to-volume ratio about 0.02 μm² μm⁻³) suggests that an additional mechanism may be responsible for regulating clot propagation under these conditions.

Shear Rate Regulates Clot Propagation from One Channel to Another Channel

To test the prediction that \dot{γ} of flowing blood regulates clot propagation, the inventors designed a microfluidic device that exposed the leading edge of a clot to flowing, re-calcified blood plasma.

FIG. 22 illustrates how a threshold to \dot{γ} regulates clot propagation through the junction. FIG. 22a is a schematic drawing of the microfluidic device used to test the dependence of clot propagation through the junction on \dot{γ}. Clot propagation through the junction was determined by monitoring three regions (dashed boxes) in the flow channel (black). Black arrows indicate the direction of flow. FIGS. 22b, c are fluorescence microphotographs of the three regions of the flow channel 27 min after the clot reached the junction. FIG. 22b shows how at \dot{γ}>\dot{γ}ₜₕᵣₑₛₕ, the clot did not propagate into the “valve”. FIG. 22c shows how, at \dot{γ}<\dot{γ}ₜₕᵣₑₛₕ,the clot propagated into the “valve” and then clotted in the rest of the flow channel down stream from the “valve”. FIG. 22d is a quantification of the dependence of clot propagation on \dot{γ}. The dashed line represents the division between short and long clot times. Solid circles represent experiments where clotting was observed in the “valve”. Open circles represent experiments stopped prior to clotting in the “valve”.

This device allowed clot initiation in the absence of flow in one channel (initiation channel, FIG. 22a) without causing initiation in the unobstructed connecting channel with flowing blood plasma. In addition, this device incorporated a geometry in the flow channel similar to a venous valve to reproduce the re-circulating flow observed in valves. FIG. 22a illustrates that this “valve” increased the residence time of the blood plasma in the flow channel and allowed monitoring of clot propagation from the junction between the initiation channel and the flow channel (subsequently referred to as the junction). Control experiments confirmed re-circulating flow in the “valve”). This system also allowed control of the average flow velocity, Vₐᵥ[m s⁻¹], and \dot{γ}. The inventors analyzed clot propagation through a junction in terms of \dot{γ}, a parameter commonly used when studying clot formation in the presence of flow. In pressure-driven flows, the local flow rate, V[m s⁻¹], at a surface is zero. Shear rate describes the change in V with increasing distance from a surface and determines transport in all directions near a surface. The inventors calculated \dot{γ} at the midpoint of the vertical wall for channels with rectangular cross-sections. A clot time was considered “long” when the time for the clot to propagate from the junction to the “valve” was greater than 30 min. FIG. 22d shows how spontaneous clotting occurred in 60-80 minutes in the flow channel.

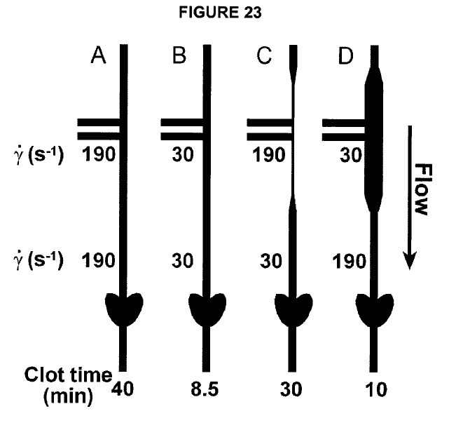

Propagation from the initiation channel to the “valve” of the flow channel showed a threshold response to \dot{γ}, with a threshold shear rate, \dot{γ}ₜₕᵣₑₛₕ, of about 90 s⁻¹ under these conditions (FIG. 22d). Clotting was initiated in the absence of flow in the initiation channel and propagated to the junction. Propagation to the junction always occurred in the absence of flow in the initiation channel. When \dot{γ} in the flow channel was above \dot{γ}ₜₕᵣₑₛₕ, clot propagation stopped at the junction, resulting in a long clot time (FIG. 22b). However, when \dot{γ} in the flow channel was below \dot{γ}ₜₕᵣₑₛₕ, the clot in the initiation channel propagated through the junction, first to the “valve” of the flow channel, and then to the rest of the flow channel downstream of the “valve”, resulting in a short clot time (FIG. 22c). At \dot{γ} very close to \dot{γ}ₜₕᵣₑₛₕ, the inventors observed both short and long clot times in two experiments with the same \dot{γ} (FIG. 23d), which demonstrated the sensitivity of propagation through a junction to \dot{γ}.

### Shear Rate at the Junction and not at the “Valve” Regulates Clot Propagation

To further demonstrate that \dot{γ} at the junction regulates clot propagation, the inventors designed devices that decoupled \dot{γ} at the junction from \dot{γ} at the “valve”. In the device shown in FIG. 22, a change in \dot{γ} at the junction resulted in a change in \dot{γ} at the “valve” and, therefore, the rate of re-circulation in the “valve”.

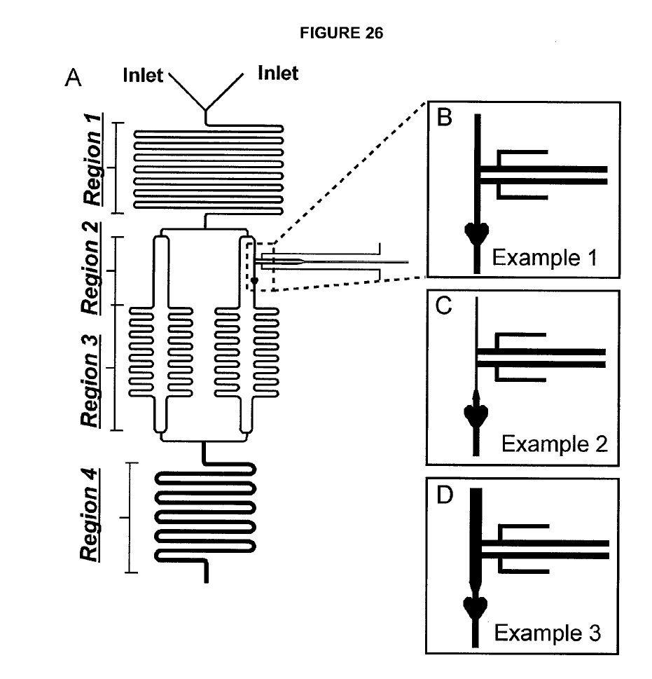

FIG. 23 illustrates how clot propagation through a junction is regulated by \dot{γ} at the junction and not at the “valve”. Shear rates, clot times, and schematic drawings of sections of the devices are shown. Clot times are reported as the average of two experiments. See FIG. 26 for device dimensions and Table 3 for flow rates for experiments in FIG. 23a-d.

A high \dot{γ} (190 s⁻¹) at both the junction and the “valve” resulted in a long clot time (FIG. 23a), while a low \dot{γ} (30 s⁻¹) at both the junction and the “valve” resulted in a short clot time (FIG. 23b). When the flow channel at the junction was narrowed to generate a high \dot{γ} at the junction and a low \dot{γ} at the “valve”, a long clot time was observed (FIG. 23c), suggesting that a low \dot{γ} at the “valve” is not sufficient to promote clot propagation through the junction. When the flow channel at the junction was expanded to generate a low \dot{γ} at the junction and a high \dot{γ} at the “valve”, a short clot time was observed (FIG. 23d), suggesting that \dot{γ} at the junction, not at the “valve”, regulates clot propagation.

TABLE 3
Flow rates and shear rates for experiments shown in FIG. 23

| | | Flow | | Flow | |
|---|---|---|---|---|---|
| | Volumetric | velocity | Shear rate | velocity | Shear rate |
| Exper- | flow rate* | junction | junction** | “valve” | “valve”*** |
| iment | (μL min⁻¹) | (mm s⁻¹) | (s⁻¹) | (mm s⁻¹) | (s⁻¹) |
| 190/190 | 2.9 | 2.4 | 190 | 2.4 | 190 |
| 30/30 | 0.5 | 0.4 | 30 | 0.4 | 30 |
| 190/30 | 2.9 | 1.1 | 190 | 0.4 | 30 |
| 30/190 | 0.5 | 0.8 | 30 | 2.4 | 190 |

*This is the volumetric flow rate in the channel with the “valve”. The volumetric flow rate in region 1 (see FIG. 26) was four times larger.
**Shear rate was calculated at the midpoint of the vertical wall for flow in a rectangular channel (Nataraja and Lakshman, 1973, Indian Journal of Technology 10: 435-438).
***Shear rate at the “valve” corresponds to the shear rate in the rectangular channel just above and below the “valve” (FIG. 26). Different shear rates in these regions correspond to different rates of re-circulation in the “valve”.

### Briefly Inhibiting Thrombin Stops Clot Propagation at Below-Threshold Shear Rates

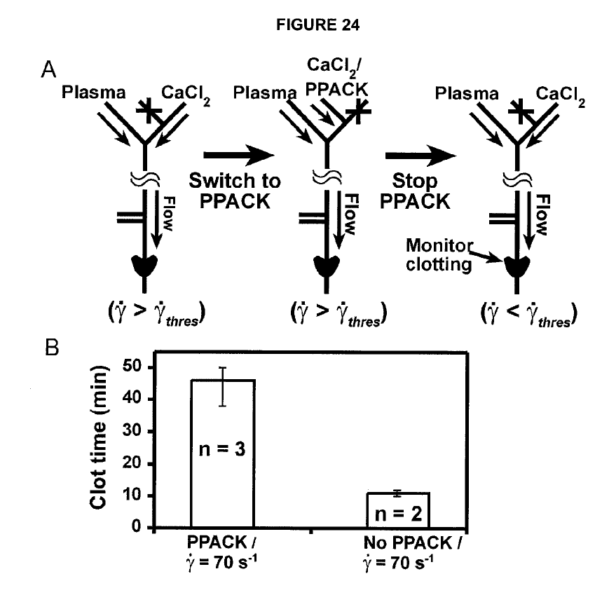

The proposed regulatory mechanism (FIG. 21) suggests that clot propagation stops at the junction when the rate of removal of activators exceeds the rate production of activators and maintains C_{act}<C_{crit} in the flow channel. Therefore, decreasing the rate of production of activator should decrease the r required maintain C_{act}<C_{crit}. To test this hypothesis, the inventors briefly exposed the clot at the junction to an irreversible direct thrombin inhibitor, D-phenylalanyl-L-prolyl-L-arginyl-chloromethyl ketone (PPACK, FIG. 24a).

FIG. 24 illustrates clot propagation through a junction when \dot{γ} in the flow channel is <\dot{γ}ₜₕᵣₑₛₕ can be reduced by briefly exposing the clot at the junction to an irreversible direct thrombin inhibitor (PPACK). FIG. 24a is a schematic drawing of an experiment in which the edge of a clot at the junction was exposed to PPACK. FIG. 24b illustrates the quantification of the effect of a seven min PPACK exposure to clot propagation through a junction when \dot{γ} in the flow channel was <\dot{γ}ₜₕᵣₑₛₕ. Clot propagation was significantly reduced after a seven min PPACK exposure. Clot times with PPACK are reported as the time after PPACK flow was stopped. Error bars are reported as the range between minimum and maximum values; average is shown.

Thrombin was selected as the target for inhibition, because it is a potent activator of clotting that is generated in high concentrations during clot propagation and participates in positive feedback. Re-calcified blood plasma was flowed into the device at \dot{γ}>\dot{γ}ₜₕᵣₑₛₕ, and clotting was initiated as in FIG. 21. When the clot reached the junction, PPACK (final concentration=0.75 μM) was incorporated into the plasma and flowed in at \dot{γ}>\dot{γ}ₜₕᵣₑₛₕ for seven minutes. Then, the flow of PPACK was stopped, re-calcified blood plasma was flowed in at \dot{γ}<\dot{γ}ₜₕᵣₑₛₕ, and clotting was monitored as in FIG. 22. This seven-minute PPACK exposure significantly slowed clot propagation from 11 min without PPACK exposure to 46 min with PPACK exposure (FIG. 24b). Control experiments in the absence of PPACK verified that the clot at the junction remained active after a 10 min exposure to \dot{γ}>\dot{γ}ₜₕᵣₑₛₕ.

These in vitro results complement previous in vivo studies which demonstrated that local administration of PPACK at sites of vascular damage required concentrations of several orders of magnitude lower than in systemic administration to achieve the same antithrombotic effect. Combined, these results suggest that irreversible direct thrombin inhibitors or reversible direct thrombin inhibitors with high binding affinities, such as hirudin (K_{d}=20 fM), could effectively prevent thrombosis through the prolonged inhibition of thrombin located in the clot.

Geometry and Dimensions of the Devices Used in Experiments where Clot Propagation at a Junction in the Presence of Flow was Monitored

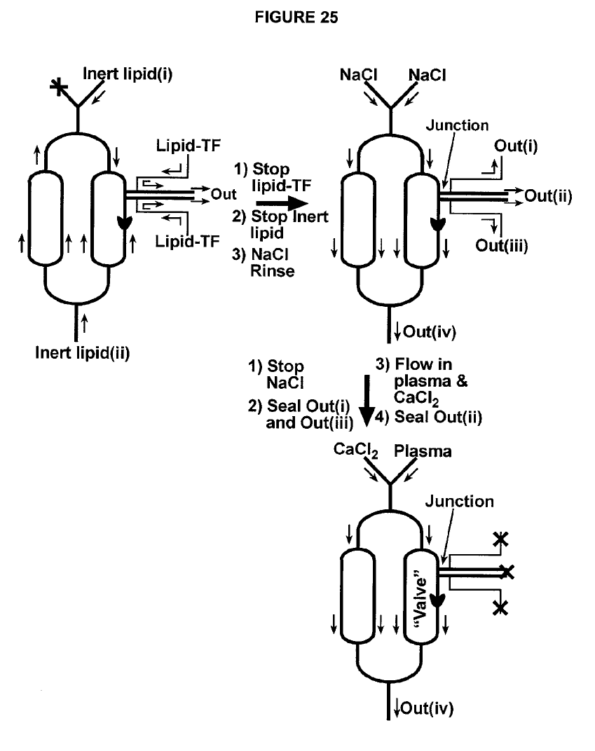

FIG. 25 is a schematic of the experimental procedure for monitoring clot propagation through a junction in the presence of flow. Shown in FIG. 25a is how two types of phospholipid vesicles (lipid-TF and inert lipid) were flowed into a PDMS device that was soaked in a solution of NaCl (150 mM). Each lipid-TF stream was flowed at 0.5 μL min⁻¹, and each inert lipid stream was flowed at 2.0 μL min⁻¹ for 15 min. To ensure that lipid-TF did not flow through the junction, the lipid vesicles were stopped in sequence. First, lipid-TF was stopped and inert lipid continued to flow for approximately one minute, To stop the inert lipid, the plugged inlet (cross) was unplugged, and a solution of NaCl (150 mM) was started at 1.0 μL min⁻¹ in this inlet. Next, the flow of inert lipid (i) was stopped, and a solution of NaCl (150 mM) was started at 1.0 μL min⁻¹ in this inlet. Finally, the flow of inert lipid (ii) was stopped. FIG. 25b illustrates how the excess lipid vesicles were removed by allowing the solutions of NaCl to flow for 20 min at 1.0 μL min⁻¹ each. This procedure left a coating of lipids on the channel walls. After the solution of NaCl was stopped, the device was removed from the solution of NaCl and Out (i) and Out (iii) were sealed (top and bottom crosses). To seal the outlets, a small amount (25-50 μL) of Norland Optical Adhesive 81 was applied to the PDMS and exposed to UV light (A=320-400 nm) for 15-20 sec. Next, blood plasma was re-calcified on chip by flowing in blood plasma and a solution of CaCl₂ (CaCl₂, 40 mM; NaCl, 90 mM; and Boc-Asp(OBzl)-Pro-Arg-MCA, 0.4 mM) at a 3:1 volumetric flow rate ratio (blood plasma:solution of CaCl₂). These solutions were allowed to flow for approximately one min and then Out (ii) was sealed as above (middle cross). Finally, the device was submerged into a solution of EDTA (50 mM). FIG. 25c illustrates how clotting initiated where the channel walls were coated with lipid-TF. This clot propagated up to the junction, and clotting was monitored in the “valve”.

FIG. 26 is a schematic drawing showing actual geometry and dimensions of the devices used for clot propagation through a junction in the presence of flow. FIG. 26a shows the basic design for the devices used in FIGS. 23, 24, and 25. For the devices in this section, the height (h), width (w), and length (l) of regions 1, 3, and 4, were the same. FIGS. 26b, c, d show variations in channel geometry made to region 2 to obtain different shear rates at the junction and the “valve” in the same experiment. The same variations were made in all four channels of region 2. For PPACK experiments (FIG. 24) the device geometry was the same as shown in a and b except that this device had one extra inlet to allow solutions to be switched.

On-Chip Titration of an Anticoagulant Argatroban and Determination of the Clotting Time within Whole Blood or Plasma Using a Plug-Based Microfluidic System

A plug-based microfluidic system was developed to titrate an anticoagulant (argatroban) into blood samples and to measure the clotting time using the activated partial thromboplastin time (APTT) test. To carry out these experiments, the following techniques were developed for a plug-based system: i) using Teflon AF coating on the microchannel wall to enable formation of plugs containing blood and transport of the solid fibrin clots within plugs, ii) using a hydrophilic glass capillary to enable reliable merging of a reagent from an aqueous stream into plugs, iii) using brightfield microscopy to detect the formation of fibrin clot within plugs and using fluorescent microscopy to detect the production of thrombin using a fluorogenic substrate, and iv) titration of argatroban (0-1.5 μg/mL) into plugs and measurement of the resulting APTTs at room temperature (23° C.) and physiological temperature (37° C.). APTT measurements were conducted with normal pooled plasma (platelet-poor plasma, PPP) and with donor's blood samples (both whole blood and platelet-rich plasma, PRP). APTT values and APTT ratios measured by the plug-based microfluidic device were compared to the results from a clinical lab at 37° C. APTT data obtained from the on-chip assay were about double of those from the clinical lab but the APTT ratios from these two methods agreed well with each other.

Reagents and Solutions. All aqueous solutions were prepared in 18-MΩ deionized water (Millipore, Billerica, Mass.). All reagents were purchased from Sigma-Aldrich (St. Louis, Mo.) unless otherwise specified. A fluorogenic substrate for human α-thrombin, t-butyloxycarbonyl-β-benzyl-L-aspartyl-L-prolyl-Larginine-4-methyl-coumaryl-7-amide (λₑₓ=365 nm, λₑₘ=440 nm), was purchased from Peptide Institute, Inc. (Osaka, Japan). For this substrate, kinetic parameters at 37° C. were kcat=160 s⁻¹, KM=11 μM in buffer solution of 50 mM Tris-HCl, pH 8.0 with 0.15 M NaCl, 1 mM CaCl₂ and 1 mg/mL BSA. The APTT reagent, Sigma Diagnostics Alexin, was obtained from Trinity Biotech (Wicklow, Ireland). Argatroban (stock concentration of 100 mg/mL) was obtained from GlaxoSmithKline (Philadelphia, Pa.). This stock was diluted with 150 mM NaCl, 20 mM Tris, pH 7.8, prior to the experiment. 1H,1H,2H,2H-perfluoro-1-octanol (PFO, 98%) was obtained from Alfa Aesar.

Protocol for the activated partial thromboplastin time (APTT) assay. Blood samples were obtained from healthy donors with approval from Institutional Review Board (protocol #12502A) by the Department of Radiology at the University of Chicago Hospitals. Whole blood was collected in vacutainer tubes at a ratio of 1 part 3.2% sodium citrate to 9 parts blood to obtain decalcified whole blood. Tubes were gently shaken to mix the contents. For experiments using donor's whole blood (which contains both cells and plasma), samples were used from the vacutainer tubes without further processing. For experiments using donor's platelet rich plasma (PRP), plasma was obtained after the samples from vacutainer tubes were centrifuged twice at 1600 rpm for 10 minutes. Normal pooled plasma (platelet-poor plasma, PPP) was obtained from George King Biomedical (Overland Park, Kans.) and stored at −80° C. These pooled plasma samples were composed of plasma from at least 30 healthy donors. For experiments using normal pooled plasma (PPP), samples were defrosted and then centrifuged at 1500 rcf for 15 minutes to remove the deposited debris resulted from prolonged storage.

The reactions in the network of blood coagulation are generally categorized into two pathways: the intrinsic pathway and the extrinsic pathway. The APTT assay measures the time required for clotting when initiated by the intrinsic pathway. APTT reagents contain two components: i) negatively charged particles that bind factor XII to initiate the intrinsic pathway, and ii) phospholipids to provide binding sites required for factor complexes. For Alexin, the APTT reagent used in this work, the activator was ellagic acid and the phospholipid was rabbit brain cephalin. First, one part of decalcified blood samples was mixed with one part of Alexin and incubated for 3 min to sufficiently activate the intrinsic pathway of coagulation. This mixture of plasma and Alexin is then recalcified with one part of 20-25 mM CaCl₂ The final concentration of CaCl₂ is about 7-8 mM. Excess CaCl₂ is used to overcome the effect of citrate. Finally, the time that elapses between the addition of CaCl₂ and the detection of fibrin clots within the sample is recorded as the APTT. This procedure was used as a guideline for adapting the plug-based microfluidic device to measure the APTT. Clinical results for the APTTs were measured with the STA Coagulation Analyzer (Diagnostica Stago, Inc., Parsippany, N.J.) by the Coagulation lab at the University of Chicago Hospital.

Microfluidic Setup. Microfluidic devices were fabricated using rapid prototyping in PDMS, poly(dimethylsiloxane). Microchannels were rendered hydrophobic and fluorophilic using the silanization protocol described previously with the exception that tridecafluoro-1,1,2,2,-tetrahydrooctyl)-1-trichlorosilane vapor was flowed into the device for 1.5 hours rather than 1 hour. In addition to the silanization protocol, the microchannels were coated with amorphous Teflon (Teflon AF 1600, poly[4,5-difluoro-2,2-bis(trifluoromethyl)-1,3-dioxole-co-tetrafluoroethylene]). First, microchannels were filled with a 1% (w/v) Teflon AF 1600 solution in a 1:4 (v/v) mixture of FC-70 and FC-3283. For experiments conducted at 37° C., microchannels were filled with a 2.5% (w/v) Teflon AF 1600 solution in a 1:1 (v/v) mixture of FC-70 and FC-3283. Then, devices were baked at 70° C. overnight until the solution evaporated. Composite glass/PDMS capillary device were fabricated as described previously (Zheng et al., 2004, Angew. Chem. Int. Edit 43: 2508-2511) with the exception that glass capillaries were rendered hydrophilic using a Plasma Prep II plasma cleaner before coupling to the PDMS device.

Microfluidic experiments. Microfluidic experiments were conducted as described previously with the following modifications. Plugs were formed using a fluorinated carrier fluid which was a mixture of 10:1 (v/v) of FC-70:PFO, where γ=10 mN m⁻¹ and p=24 mPa s at 23° C. Flow rate of the fluorinated carrier fluid was maintained at 3 μL/min. Aqueous solutions used to form plugs were Alexin and blood samples (which were either whole blood, platelet-rich plasma or platelet-poor plasma, more information in the next paragraphs). For Alexin, the flow rate was 0.3 μL/min for experiments conducted at 23° C. and 1.2 μL/min for experiments conducted at 37° C. For the two blood streams, the total flow rate was 0.3 μL/min for 23° C. and 1.2 μL/min for 37° C. A droplet of 100 mM CaCl₂ solution (300 mOs) was injected into each plug at the merging junction. The flow rate of the CaCl₂ solution was 0.2 μL/min for 23° C. and 0.4 μL/min for 37° C. Estimated from the flow rate of the Alexin, the two blood streams and the CaCl₂ solution, the concentration of CaCl₂ was 25 mM and 14 mM for experiments at 23° C. and 37° C. respectively. Excess CaCl₂ was used to overcome the effect of citrate. For experiments at 37° C., a microscopic heating stage (Brook Industries, Lake Villa, Ill.) was used to keep the devices at 37° C.

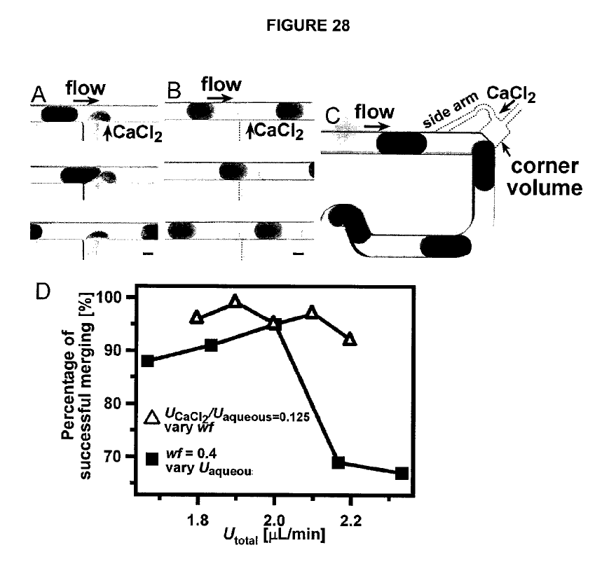

In all figures in this section (except in FIG. 28), the main PDMS channel of the microfluidic device was 300 μm×270 μm (width×height), the small channel was 100 μm×100 μm. In FIG. 28a, the main PDMS channel and the side channel both were 200 μm×250 μm. In FIG. 28b, the main PDMS channel was 200 μm×250 μm, the small side channel was 50 μm×50 μm. In FIG. 28c, the main PDMS channel was 200 μm×260 μm, the height of the side arm and the corner volume was 80 μm.

Measurement of the APTT with whole blood samples. For microfluidic experiments with whole blood, the stock solutions in the aqueous syringes were i) Alexin, ii) whole blood and iii) whole blood with 3.0 μg/mL argatroban. Experiments were conducted using either a Leica DM IRB or DMI6000 microscope. Fibrin clots within plugs formed with whole blood were detected optically using a Spot Insight color digital camera (Diagnostics Instruments, Inc).

Measurement of the APTT with plasma samples. For microfluidic experiments with plasma (either platelet-rich or platelet-poor), the stock solutions in the three aqueous syringes were i) Alexin, ii) plasma with 150 μM. fluorogenic substrate, prepared by adding 3.5 μL substrate solution into 246.5 μL plasma, and iii) plasma with 150 μM fluorogenic substrate and 3.0 μg/mL argatroban, prepared by adding 3.5 μL substrate solution and 0.75 μL of argatroban (1 mg/mL) into 245.5 μL plasma. Experiments were conducted using a Leica DMI6000 microscope. Cleavage of the fluorogenic substrate for α-thrombin was monitored on the microscope by fluorescence, using a DAPI filter (λₑₓ=350±25 nm, λₑₘ=460±25 nm) and a cooled CCD ORCA ERG 1394 (12-bit, 1344×1024 resolution) (Hamamatsu Photonics, K. K., Hamamatsu City, Japan). Fibrin clots within plasma samples was monitored on the microscope by brightfield microscopy.

### Overall Design of the Microfluidic Chip for Performing APTT Test

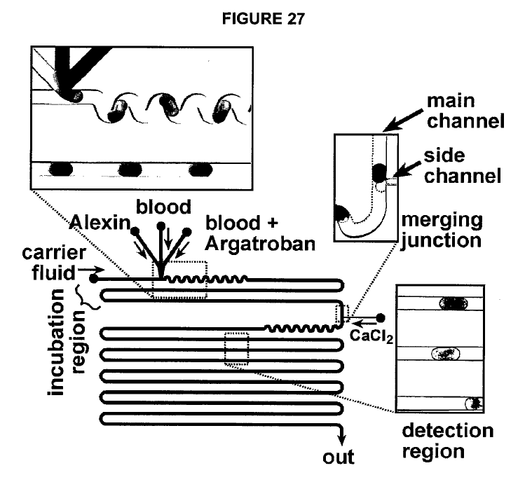

The microfluidic device consisted of five different regions: the plug-forming region, the mixer, the incubation region, the merging junction and the detection region (FIG. 27). Shown in FIG. 27 is a schematic of a plug-based microfluidic device for determining the APTT and for titrating argatroban. Plugs containing Alexin (the APTT reagent) and blood (either plasma or whole blood) were formed in the plug-forming region, which were then transported to the incubation region (microphotograph, upper left). After flowing for 3 minutes, CaCl₂ solution was injected into each plug at the merging junction (microphotograph, upper right). The CaCl₂ droplet was traced with a dashed line in the microphotograph. In the detection region, clots formed within plugs were observed as a function of time (microphotograph, lower right).

Plugs of the three aqueous reagents were formed: i) Alexin, ii) decalcified blood and iii) decalcified blood mixed with argatroban. The blood sample was either donor's whole blood, donor's plasma (PRP) or normal pooled plasma (PPP). The flow rate of the Alexin and the combined flow rate of the blood streams were maintained at a 1:1 ratio, as required by the APTT assay. By varying the relative flow rates of the two blood streams, the concentration of argatroban within plugs was varied. Winding channels were incorporated into the design of the microfluidic network to promote mixing of the reagents within plugs. The length of microchannel in the incubation region was specifically designed so that at the total flow rate of the aqueous and fluorinated carrier fluid streams, the incubation time of the plugs was 3 minutes, as specified by the APTT assay (FIG. 27, upper region of microchannel network).

The merging junction was required to inject CaCl₂ into the plug after incubation (FIG. 27, right side of microchannel network). More information about this junction is given below. To accelerate mixing of CaCl₂ within the plug, another winding channel was designed into the microchannel network. The starting time of the APTT (t=0) was established when the plugs of blood were merged with the CaCl₂ solution at the merging junction. This is consistent with the one used in clinical laboratories where the starting time of the APTT assay equals the time of addition of CaCl₂ to the blood sample. However, in the preliminary microfluidic experiments, the clotting time appeared to be dependent on the rate of mixing. The rate of mixing is known to affect a wide range of autocatalytic systems.

For more reliable transport of the fibrin clots inside the plugs without sticking to the PDMS microchannel wall, the surface of the microchannel was first treated with fluorinated silane and then coated with amorphous Teflon. To determine the time at which fibrin clots formed within the plug, images were taken and analyzed by brightfield and fluorescence microscopy in the detection region (FIG. 27, lower region of the microchannel network).

Two New Methods of Merging a Stream into Flowing Plugs

To perform a multi-step assay on a plug-based microfluidic system, injection of reagents into a plug is necessary. Three merging methods were previously developed for plug-based microfluidics: i) the reagent was directly injected into a plug as it moved past the channel containing the reagent; ii) a small droplet was merged into an adjacent larger plug in the main channel when the frequency was matched between formation of the droplet and of the plug₃₅; iii) ten smaller droplets were merged into a single larger plug. However, these three methods were difficult to implement in this assay. At these slow flow rates (0.1 to 0.2 mm/s for the CaCl₋₂ stream), contamination of the CaCl₂ stream occurred in the side channel when CaCl₂ was directly injected into the passing plug (FIG. 28a). If a side junction was used with a smaller width and height, small droplets of CaCl₂ formed and did not merge with the passing plug at the junction (FIG. 28b).

FIG. 28 illustrates merging within a microfluidic device using a hydrophobic side channel. Shown in FIG. 28a is how when the side channel was hydrophobic (silanized PDMS), contamination occurred (for 6 out of 5 experiments) when the side channel was large (width of 200 μm and height of 250 μm). FIG. 28b illustrates that merging did not occur (for 4 out of 4 experiments) when the side channel was too small (width and height of 20 μm). Another approach for merging was to form droplets of CaCl₂ at the same frequency as the passing plug. FIG. 28c shows how at the junction, the carrier fluid between the passing plugs flows into the side arm to break off a droplet from the CaCl₂ stream. Shown in FIG. 28d is that consistent merging was obtained for U_{CaCl2}/U_{aqueous}=0.125 at various water fraction wf (Δ). At a constant wf=0.4, high percentage of merging (95%) was measured only for U_{CaCl2}/U_{aqueous}=0.125 (▪). Each symbol represents measurements from 100 plugs. All scale bars are for 100 μm.

The inventors implemented two new approaches for merging.

For the first approach, the merging junction was designed so that the fluorinated carrier fluid between the plugs flowed into the side arm to break off a droplet of CaCl₂ within the corner volume (FIG. 28c). To make this design, the size of the aqueous plug and the carrier fluid spacing between plugs was characterized for various water fraction, wf. Using this design, the frequency was matched between the plug passing that junction and the droplet forming at the corner volume. Successful merging was dependent on the ratio of U_{CaCl2}/U_{aqueous} and not on the water fraction wf. Water fraction, wf=U_{aqueous}/Uₜₒₜₐₗ where U_{aqueous}[μL/min] is the total volumetric flow rates of the aqueous streams for blood and Alexin, Uₜₒₜₐₗ[μL/min] is the total volumetric flow rates of the blood, Alexin and carrier fluid streams, and U_{CaCl2}[μL/min] is the flow rate of the CaCl₂ stream. There was a dependence of length of plugs and carrier fluid spacing between plugs as a function of wf and Uₜₒₜₐₗ[μL/min]. For wf=0.4, the highest percentage of successful merging events (95%) was observed when U_{CaCl2}/U_{aqueous}=0.125, where U_{CaCl2} was maintained at 0.1 μL/min (FIG. 2d, solid symbols). If U_{CaCl2}/U_{aqueous} was maintained at 0.125, then successful merging (92% to 99%) was observed for various wf from 0.36 to 0.45 (FIG. 2d, open symbols). The advantage of this approach was that it did not require extensive fabrication effort. However, merging did not occur consistently over a wide range of U_{CaCl2}/U_{aqueous}.

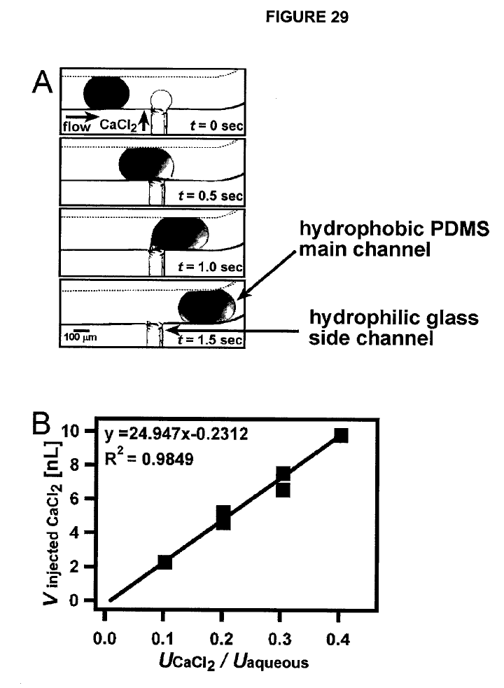

FIG. 29a illustrates consistent merging with a hydrophilic glass capillary inserted into the side channel. FIG. 29b shows how the injection volume of CaCl₂, V_{injected CaCl2}[nL], into the plug was controlled by flow rate [μL/min], where U_{CaCl2} was the flow rate of the CaCl₂ stream and U_{aqueous} was the total aqueous flow rate for streams of Alexin and blood. In the graph, each symbol represents measurements for 10 plugs. At least two symbols are shown for each value of U_{CaCl2}/U_{aqueous}, where some symbols coincide.

The approach shown in FIG. 29a relied on control of surface chemistry of the side channel. A small side channel was used to avoid back-contamination (as in FIG. 28b) but it was made hydrophilic. The merging junction was fabricated by inserting a hydrophilic capillary into this side channel. The solution of CaCl₂ remained attached to the capillary due to wetting and the undesirable droplets seen in FIG. 28b did not form. In this example it is important to: (i) insert the capillary flush with the edge of the main channel for this method to work, and (ii) have size of blood plugs larger than size of CaCl₂ droplet (U_{CaCl2}/U_{aqueous}<1, typically 0.17-0.33 in experiments here). When these two requirements were satisfied, consistent merging (100%, >40 experiments in different devices) was observed at the flow rates of the aqueous streams (0.6-2.4 μL/min) and the CaCl₂ stream (0.2-0.4 μL/min) the inventors used for APTT assay. The volume of CaCl₂ being injected into the plug, V_{injected CaCl2}[nL], linearly increased with the U_{CaCl2}/U_{aqueous} (FIG. 29b). By controlling the flow rates, the exact amount of the injecting reagent could be easily controlled. This merging approach was used for direct injection of CaCl₂ solution for APTT measurement.

Detecting Clots within Plugs and Analyzing Images to Measure the APTT and Thrombin Generation

The APTT is the elapsed time from the addition of CaCl₂ and to the detection of fibrin clots within the blood sample, In most point-of-care devices and commercially available machines used in testing centers, formation of the fibrin clot is detected by detecting changes in optical transmittance or in movement of magnetic particles. Here, fibrin clots within plugs were detected by brightfield and thrombin generation within plugs was detected by fluorescence microscopy. By analyzing images taken of plugs traveling through the microchannel, the inventors established a standardized method to determine the APTT in plugs.

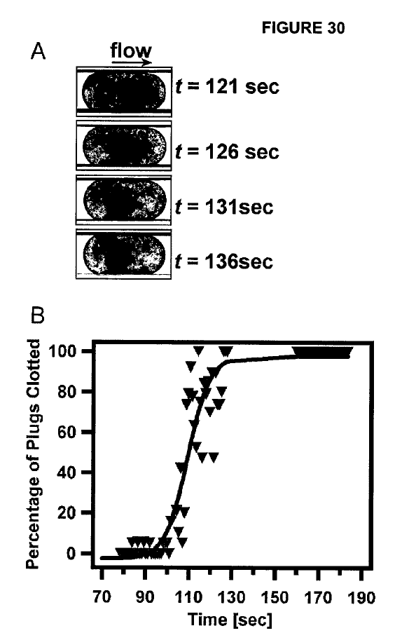

Detecting fibrin clots in plugs of donor's whole blood. For plugs formed with whole blood, brightfield microscopy was used to detect the trapping of red bloods cells (RBCs) within fibrin clots. FIG. 30 illustrates using brightfield microscopy to observe clots within plugs of whole blood. FIG. 30a illustrates how a single plug of whole blood was followed as it traveled through the microchannel. Time t[sec] was time for the plug traveled after merging with CaCl₂. Whole blood within the plug was considered fully clotted when red blood cells were no longer moving inside the plug and a dense clot was observed within the back half of the plug (a, bottom image).

FIG. 30b illustrates how, by analyzing images of plugs (like in a), the percentage of plugs that contained fibrin clots was determined for each time point in the detection region. A total of at least 20 plugs were used for each time point. Experiments were performed at 23° C.

The APTT was determined to be the time at which the RBCs within the plug were no longer moving (relative to the motion of the plug flowing through the microchannel). Series of images of a single plug were acquired at 2 frames/sec. To follow a single plug, the microscope stage was moved at the same speed relative to the speed of the plug moving through the microchannel. Before clotting, the RBCs were evenly distributed and were moved by internal circulation within the plugs After some time, small clumps of RBCs appeared within the plug but other RBCs still moved by internal circulation (FIG. 30a, top image, t=121 sec). The shear (about 2 s⁻¹) within moving plugs was much lower than that required to induce clotting by activating platelets (about 750 s⁻¹). At a later time, a larger and denser clump of RBCs trapped in a fibrin clot moved to the back half of the plug while the rest of the RBCs did not move due to being trapped within the fibrin network (FIG. 30a, bottom image, t=136 sec). For the plug shown in FIG. 30a, the APTT of the plug was t=136 sec at 23° C. tₜᵣₐₙₛ[s] was defined as the time that elapses from the first sign of clotting (FIG. 30a, top image) to when the RBCs no longer move relative to the plug (FIG. 30a, bottom image). For this plug shown in FIG. 30a, tₜᵣₐₙₛ was 15 sec

The APTT was also determined from many plugs statistically. At each time point, images were acquired for at least 20 plugs. From a set of images at each time point, the number of plugs that contain fibrin clots was counted. This number was divided by the total number of plugs to obtain the “percentage of plugs clotted” at each time point (FIG. 30b). The APTT was the time for 50% of plugs of whole blood to be clotted. The APTT was 122 sec at 23° C. (FIG. 30b), in agreement with previously measured APTTs of 175±58 sec at 23° C. and 104±20 sec at 25° C. The average tₜᵣₐₙₛ was 15.4±2.8 sec for 9 plugs of whole blood.

Detecting clots within plugs formed with donor's plasma (platelet-rich). Clinical labs frequently measure the APTT using plasma, rather than whole blood. The inventors determined the APTT in plasma with two methods: using brightfield microscopy to observe formation of dense fibrin clots and using fluorescent microscopy to detect cleavage of a fluorogenic substrate by thrombin.

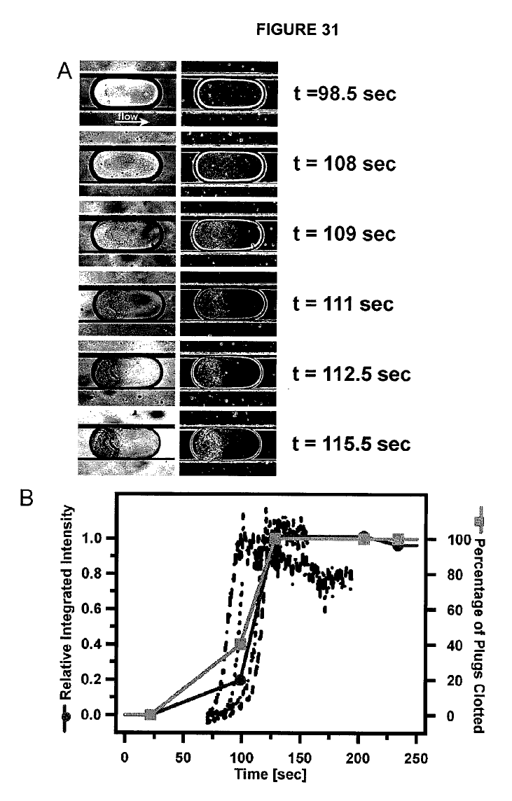

FIG. 31 illustrates using brightfield and fluorescence microscopy to observe the formation of fibrin clots within plugs of platelet-rich plasma (PRP). FIG. 31a shows how a single plug of plasma was followed as it traveled through the microchannel (a, left panels). Brightfield images were processed with a digital Sobel filter to see clots more easily (a, right panels). Plasma was considered fully clotted when the fibrin clot condensed into the back half of the plug and sequential images of the plug looked the same (compare image at t=112.5 sec to image at t=115.5 sec). FIG. 31b illustrates how plugs were formed containing a fluorogenic substrate for thrombin in plasma. The fluorescence intensity of the substrate increases. In the graph, each black dashed line represents the fluorescence intensity arisen from an individual plug, where a single plug was followed as it traveled through the microchannel (total of 4 plugs are shown). Integrated intensities obtained from images collected with fluorescence microscopy was compared to (red square) the percentage of plugs clotted observed from images with brightfield microscopy. About 50% of the plugs were clotted when the fluorescence intensity was about 30% of the maximum fluorescence signal. Each symbol represents the measurement of at least 10 plugs at each time point in the detection region. Experiments were performed at 23° C.

To observe fibrin clots in plasma using brightfield microscopy, a time series of images was acquired for a single plug traveling through the microchannel (FIG. 31a, left panels). A digital convolution filter Sobel (from Metamorph software) was used to aid the visual detection of the clot (FIG. 31a, right panels). For the plug shown in FIG. 31a, the APTT was about 113 sec and tₜᵣₐₙₛ was 14 sec. tₜᵣₐₙₛ[s] was defined as the period of time that elapses from the first sign of clotting (FIG. 31a, first image) to when the fibrin clot no longer moves relative to the plug (FIG. 31a, fifth image).

Using fluorescence microscopy, a more quantitative determination of the thrombin generation can be made for plugs of plasma. The inventors used a fluorogenic substrate for thrombin. When cleaved by thrombin, the fluorescence intensity of the substrate increases by about 10-fold. Thrombin is the final enzyme produced in the coagulation network and it drives formation of the fibrin clot by cleaving fibrinogen. Fibrin clots form at low concentrations of thrombin (2-10 nM) while the majority of the thrombin (about 1 μM) is produced after the clot is fully formed. Thrombin favors cleaving fibrinogen compared to the substrate.

A single plug of plasma was followed as it traveled through the microchannel and the fluorescence intensity was measured as a function of time (as shown for four plugs, each plug represented by one black dashed line, FIG. 31b). Although the actual APTT of each individual plugs was different, the time taken for the relative fluorescence intensity to increase from 0 to 1 was the same. To determine the average APTT for many plugs, the inventors correlated the detection of fibrin clots by brightfield microscopy to the detection of thrombin generation by fluorescence microscopy. Images were acquired at each time point by brightfield and fluorescence microscopy from the same experiment. Brightfield images were analyzed to determine the percentage of plugs clotted as a function of time. The APTT (about 100 sec) was determined to be the time at which 50% of the plugs contained fibrin clot. This APTT correlated to a fluorescence intensity of about 30% of the maximum fluorescence signal (FIG. 31b).

### Titration of Argatroban and Measurement of the APTT and Thrombin Generation

To determine the effect of the anticoagulant on the APTT, APTTs were measured while argatroban was titrated into samples of normal pooled plasma, donor's plasma or donor's whole blood. Measuring the APTT of normal pooled plasma is a standard calibration procedure for coagulation instruments in central clinical labs. Therefore, the inventors also obtained APTTs from normal pooled plasma. For on-chip titration, one of the two inlet streams of blood contained 3 μg/mL of argatroban. By varying the relative flow rates of these two blood streams, the concentration of argatroban within the plugs was varied. Experiments were conducted at 23° C. and 37° C.

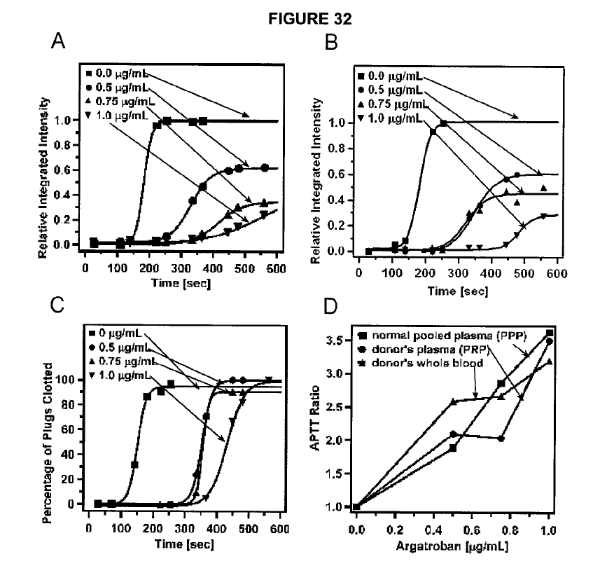

FIG. 32 illustrates measurement of thrombin generation and APTT at 23° C. while titrating argatroban into blood samples. FIGS. 32a, b illustrates the detection of thrombin generation in plasma. FIG. 32c shows the measurement of APTT in whole blood. FIG. 32d shows the resulting APTT ratios for (c). The concentration of argatroban within the plugs was 0 μg/mL, 0.5 μg/mL, 0.75 μg/mL and 1.0 μg/mL. Each symbol represents the measurement of at least 20 plugs. Shown in FIG. 32c, for whole blood samples, the APTT was the time at which the percentage of plugs clotted was 50%. FIG. 32d illustrates how the APTT ratio was determined for the whole blood samples at each concentration of argatroban. The APTT ratio was the ratio of the APTT with argatroban to the baseline APTT without argatroban.

For experiments conducted at 23° C., the effect of argatroban on thrombin generation for the donor's plasma samples agreed satisfactorily with the results from the normal pooled plasma (FIGS. 32a,b). The APTT ratio is the ratio of the APTT with argatroban in plasma to the baseline APTT without argatroban. For the donor's whole blood samples, the APTT ratio at 23° C. showed a dependence on the concentration of argatroban (FIG. 32d). Generally, doses of argatroban between 0.2 and 2.0 μg/mL are required to achieve an APTT ratio between 1.5 and 3.0. Using this on-chip APTT assay, an APTT ratio of 2.3 was reached for an argatroban dose of 0.5 μg/mL and an APTT ratio of 2.8 for an argatroban dose of 1.0 μg/mL at 23° C. (FIG. 32d). For this donor, a non-linear dependence of the APTT ratios on the concentration of argatroban was observed. This dependence was reproducible from experiments with plasma to experiments with whole blood.

Two modifications from the protocol were required to conduct experiments at the physiological temperature of 37° C. First, a more concentrated Teflon AF solution (2.5% w/v instead of 1% w/v for 23° C. measurements) was used to coat the microchannel to prevent the sticking of fibrin clots onto the microchannel walls. Fibrin clots were more likely to attach to the walls of channel at higher temperatures. Second, a higher injection flow rate of the Alexin and blood sample was used to form larger plugs (the width-to-length ratio of the plug was about 1:3).

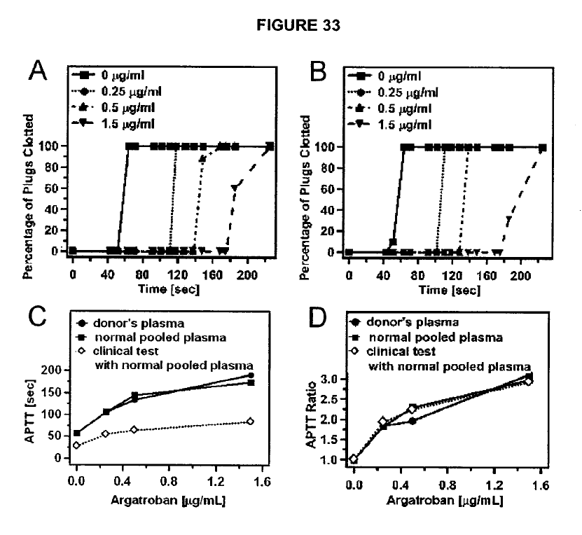

FIG. 33 illustrates APTT measurements at 37° C. while titrating argatroban into (a) normal pooled plasma, (b) donor plasma and corresponding values of the (c) APTT and (d) APTT ratios. For both plasma samples, the APTT was the time at which 50% of plugs contained fibrin clot. The concentration of argatroban within the plugs was 0 μg/mL, 0.25 μg/mL, 0.5 μg/mL and 1.5 μg/mL. Each symbol represents the measurement of at least 20 plugs. FIG. 33c illustrates how the values of the clinical APTTs with normal pooled plasma were about 2 times lower than the APTTs measured with the plug-based microfluidic experiments with normal pooled plasma and donor's plasma. FIG. 33d shows how the APTT ratios agreed well among the clinical APTTs with normal pooled plasma and the plug-based microfluidic experiments with normal pooled plasma and donor's plasma.

While titrating argatroban in the same manner as the 23° C. experiments, APTTs were measured for normal pooled plasma (FIG. 33a) and donor plasma (FIG. 33b) at 37° C. APTTs obtained at 37° C. were also about 2.5 times shorter than those at 23° C. APTT ratios were similar at these two temperatures. Argatroban of 0.5 μg/mL resulted an APTT ratio of 2.3 at 23° C. (FIG. 6d) and an APTT ratio of about 2.1 at 37° C. (FIG. 33b). Argatroban of 1.0 μg/mL resulted an APTT ratio of 2.8 at 23° C. (FIG. 32d) and an APTT ratio of 2.7 at 37° C. (FIG. 33d). APTT values and APTT ratios measured by the on-chip assay at 37° C. were compared to results from a clinical lab at 37° C. Pooled plasma samples were mixed with argatroban (0-1.5 μg/mL) and submitted to the Coagulation lab at the University of Chicago Hospital for APTT measurements. APTTs obtained from the Coagulation lab were consistently about half of what the inventors obtained from the on-chip assay (FIG. 33c). However, the corresponding APTT ratios from these two methods agreed closely to each other (FIG. 33d).

Two technical developments enabled the work presented in this example. First, the use of a Teflon AF coating helped minimize sticking of fibrin clots on the walls of microchannels. Second, reliable addition of a reagent from an aqueous stream into plugs was achieved by injecting the reagent stream through a hydrophilic narrow glass capillary. This merging method would be important for performing multi-step assays and reactions in plugs, especially when cross-contamination must be minimized and ratios of reagents must be varied. The methods of this invention would be useful for other assays using blood, including the Prothrombin Time (PT) assay and the detection of other analytes within the blood samples. Rapidly performing multiple tests and titrations on a single blood sample using preloaded reagent cartridges (Zheng et al., 2005, Angew. Chem. Int. Edit. 44: 2520-2523) is an exciting opportunity that can be realized with this plug-based microfluidic system.

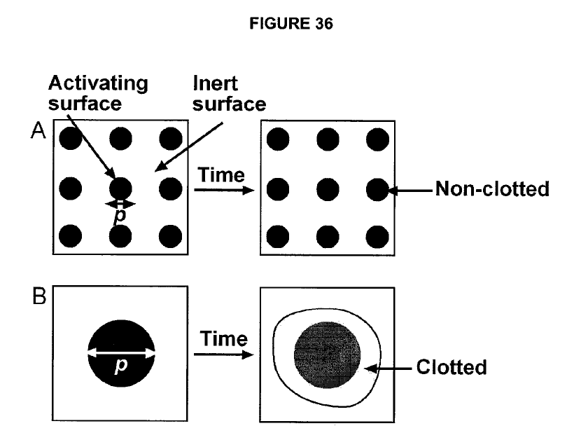

FIG. 36 is a schematic of an experiment to test the hypothesis that the size of individual patches, p, is important, not the total surface area.

FIG. 36a illustrates the hypothesis that an array of small patches (pₛ) of an activating surface will not initiate clotting. FIG. 36b illustrates how a single large patch (pᵢ) will initiate clotting. The total activating surface area of the nine patches in (a) is equal to that of the large patch in (b). The activating surface is an acidic layer for chemical model experiments and negatively charged lipids containing tissue factor for blood plasma experiments.

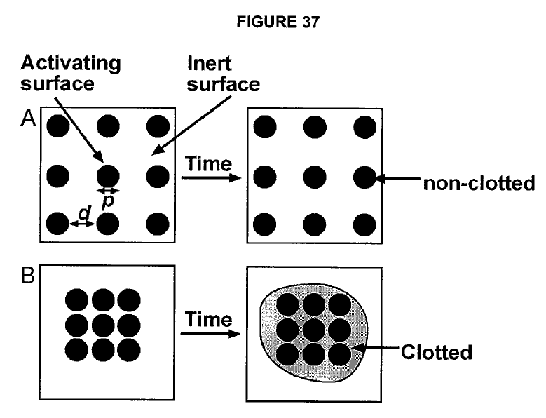

FIG. 37 is a schematic of an experiment to test the hypothesis that a cluster of sub-threshold patches will initiate clotting when they are brought close enough together to communicate by diffusion. FIG. 37a illustrates the hypothesis that a cluster of sub-threshold patches of an activating surface will not initiate clotting when they are separated by a distance, d, greater than the diffusion length scale pₜᵣ. FIG. 37b shows how sub-threshold patches should initiate clotting when they are separated by a distance that is shorter than pₜᵣ. The activating surface is an acidic layer for chemical model experiments and negatively charged lipids containing tissue factor for blood plasma experiments.

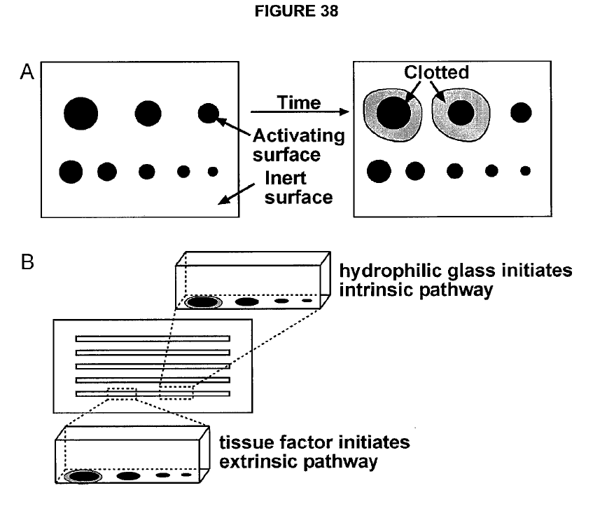

FIG. 38 illustrates the schematic of a system capable of rapidly characterizing a person's clotting potential. FIG. 38a illustrates a single array of patches of different sizes that can be used to rapidly measure the threshold patch size for a particular blood sample. Two types of activating surfaces can be used, negatively charged lipids with reconstituted TF (for extrinsic pathway), and hydrophilic glass (for intrinsic pathway). FIG. 38b illustrates how arrays of patches can be fabricated inside microfluidic channels. Each channel can contain a series of tissue factor patches and a series of hydrophilic glass patches. Between channels, parameters such as the range of patch sizes, TF concentration, and drug dosage can be varied. High-throughput measurements can be done for large numbers and types of samples, including commercially available plasma samples with clotting factor abnormalities, and blood samples with added drugs, such as argatroban and heparin.

It is to be understood that this invention is not limited to the particular devices, methodology, protocols, subjects, or reagents described, and as such may vary. It is also to be understood that the terminology used herein is for the purpose of describing particular embodiments only, and is not intended to limit the scope of the present invention, which is limited only by the claims. Other suitable modifications and adaptations of a variety of conditions and parameters normally encountered and obvious to those skilled in the art, are within the scope of this invention. All publications, patents, and patent applications cited herein are incorporated by reference in their entirety for all purposes. Also incorporated by reference in their entirety for all purposes are the supplementary materials (including information, text, graphs, images, tables, and movies) available online, and associated with some of the above-referenced publications.

What is claimed is:

1. An apparatus for assaying clotting activity comprising:
an inlet for a blood fluid;
a vessel in fluid communication with the inlet; and
at least a first and a second patch in the vessel, wherein
(a) the patches each comprise stimulus material which is capable of initiating a clotting pathway when contacted with a blood fluid from a subject; and
(b)(i) the stimulus material in the first patch differs from the second patch; or
(b)(ii) the concentration of stimulus material in the first patch differs from the second patch; or
(b)(iii) the first patch has a surface area different from the second patch; or
(b)(iv) the first patch has a shape different from the second patch; or
(b)(v) the first patch has a size different from the second patch.

2. The apparatus of claim 1, comprising a plurality of patches.

3. The apparatus of claim 2, wherein the distance between the members of a first set of two patches is different from the distance between the members of a second set of two patches.

4. The apparatus of claim 2, wherein a first set of patches is at a first location and a second set of patches is at a second location; and wherein the number of patches in the first set is different from the number of patches in the second set.

5. The apparatus of claim 1, wherein the stimulus material comprises at least one clotting stimulus selected from the group of tissue factor, factor II, factor XII, factor X, glass, glasslike substances, kaolin, dextran sulfate, amyloid beta, ellagic acid, bacteria, and bacterial components.

6. The apparatus of claim 1, wherein the patches are beads.

7. The apparatus of claim 1, further comprising beads, wherein the patches are associated with the beads.

8. The method of claim 1, wherein the patch further comprises inert material.

9. The apparatus of claim 1, wherein the vessel comprises two intersecting microchannels, and wherein the channels are in fluid communication with each other.

10. A method of assaying blood clotting, comprising
contacting blood fluid from a subject with at least a first and second patch, wherein
(a) the patches each comprise stimulus material which is capable of initiating a clotting pathway when contacted with a blood fluid from a subject; and
(b)(i) the stimulus material in the first patch differs from the second patch; or
(b)(ii) the concentration of stimulus material in the first patch differs from the second patch; or
(b)(iii) the first patch has a surface area different from the second patch; or
(b)(iv) the first patch has a shape different from the second patch; or
(b)(v) the first patch has a size different from the second patch; and
determining which patches initiate clotting of the blood fluid from the subject.

11. The method of claim 10, wherein the stimulus material is capable of initiating a clotting pathway in blood fluid from a healthy subject.

12. The method of claim 10, wherein the contacting is for a time sufficient for at least the largest patch to initiate the clotting pathway in a blood fluid from a healthy subject.

13. The method of claim 10, further comprising a surface in which the patches are associated.

14. The method of claim 13, further comprising contacting blood fluid from the subject with a third patch associated with the surface, and wherein the distance between the first and second patches differs from the distance between the second and third patches.

15. The method of claim 13, wherein the surface is a microfluidic channel.

16. The method of claim 15, wherein the blood fluid is contacted with the patches in plugs separated by an immiscible fluid.

17. The method of claim 15, wherein the blood fluid is contacted with the patches as a continuous stream.

18. The method of claim 10, wherein the patches are each independently a bead.

19. The method of claim 10, wherein the patches are each independently associated with a bead.

20. The method of claim 18, wherein either the size or the shape of each beads differ.

21. The method of claim 10, wherein the clotting pathway is a platelet aggregation pathway.

22. The method of claim 10, wherein contacting comprises first contacting a first amount of blood fluid with a first concentration of beads and second contacting a second amount of blood fluid with a second concentration of beads; wherein each bead independently is associated with a patch comprising a stimulus material and an inert material.

23. The method of claim 21, wherein aliquots of blood fluid are titrated with beads of increasing size.

24. The method of claim 10, wherein determining comprises observing optically.

25. The method of claim 10, wherein determining comprises measuring scattering of light.

26. The method of claim 10, wherein the blood fluid is selected from the group consisting of whole blood, blood constituents, plasma, a solution of plasma proteins, and a solution of cells from blood.

27. The method of claim 10, further comprising first adding an excess of a clotting factor to the blood fluid before contacting the blood fluid with the patches.

28. The method of claim 10, further comprising adding a test substance to a blood fluid before contacting the blood fluid with the patches.

29. The method of claim 10, further comprising monitoring the rate of propagation of a blood clot.

30. The method of claim 10, further comprising adding a blood fluid from a different subject to the blood fluid before contacting the blood fluid with the patches.

31. An apparatus for measuring clot propagation comprising:
a first region comprising a stimulus material; and a second region in communication with the first region suitable for monitoring the propagation of a clot; wherein when a blood fluid is placed in the first region, a clot forms and propagates to the second region.

32. The apparatus of claim 31, further comprising a patch comprising the stimulus material.

33. The apparatus of claim 31, wherein the apparatus comprises a microchannel comprising the first and second regions.

34. The apparatus of claim 31, wherein the apparatus comprises a plurality of microchannels, each microchannel comprising separate first and second regions.

35. The apparatus of claim 31, comprising at least one set of intersecting microchannels, wherein the second region is at the intersection of the first set of the microchannels.

36. The apparatus of claim 35, comprising a plurality of microchannels and at least two intersections of the microchannels, wherein the second region is at one of the intersections and wherein the sizes of the two intersections are different.

37. A method of monitoring clot propagation, comprising the steps of:
contacting a blood fluid with a first region of an apparatus, the first region comprising a stimulus material, and
monitoring clot propagation in a second region of the apparatus, the second region in communication with the first region.
